(19) United States
(12) Patent Application Publication
(10) Pub. No.: US 2025/0360229 A1
(43) Pub. Date: Nov. 27, 2025

(54) SYSTEMS, METHODS, AND COMPOSITIONS FOR TREATING VASCULAR DISEASE

(71) Applicants: The Broad Institute, Inc., Cambridge, MA (US); The Board of Trustees of the Leland Stanford Junior University, Stanford, CA (US); The Brigham and Women's Hospital, Inc., Boston, MA (US)

(72) Inventors: Gavin Reinhardt SCHNITZLER, Cambridge, MA (US); Jesse Michael ENGREITZ, Stanford, CA (US); Helen Yihua KANG, Stanford, CA (US); Xueyan Rosa MA, Stanford, CA (US); Rajat Mohan GUPTA, Boston, MA (US)

(73) Assignees: The Broad Institute, Inc., Cambridge, MA (US); The Board of Trustees of the Leland Stanford Junior University, Stanford, CA (US); The Brigham and Women's Hospital, Inc., Boston, MA (US)

(21) Appl. No.: 19/190,085

(22) Filed: Apr. 25, 2025

Related U.S. Application Data
(63) Continuation of application No. PCT/US2023/077860, filed on Oct. 26, 2023.
(60) Provisional application No. 63/419,997, filed on Oct. 27, 2022.

(51) Int. Cl. A61K 48/00; C12N 9/22; C12N 15/11; C12Q 1/6883

(52) U.S. Cl. CPC A61K 48/0058; C12N 9/22; C12N 15/11; C12Q 1/6883; C12N 2310/20; C12Q 2600/156

(57) ABSTRACT

Provided herein are methods and compositions for the diagnosis, prognosis, and treatment of a vascular disease, such as coronary artery disease (CAD), in a subject. In particular, provided are methods and compositions for treating a vascular disease in a subject involving administering a therapy to disrupt the cerebral cavernous malformation (CCM) signaling pathway in endothelial cells (e.g., arterial endothelial cells) in the subject. Also provided are methods of determining the likelihood that a subject will respond to a therapy for a vascular disease such as CAD, based on the identification of one or more loss-of-function variants in a CCM pathway associated gene in the subject.

### CROSS-REFERENCE TO RELATED APPLICATIONS

This application is a Continuation of International Application No. PCT/US2023/077860, filed Oct. 26, 2023, and claims the benefit under 35 U.S.C. § 119(e) of U.S. Provisional Patent Application No. 63/419,997, filed Oct. 27, 2022, the entire contents of which are incorporated herein by reference in their entireties.

### STATEMENT OF U.S. GOVERNMENT SUPPORT

This invention was made with government support under Grant Nos. HL152423, HL159176, HL164811, HG011324, HG009917, and HG011972 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Aug. 15, 2025, is named 114203-10502_SL.xml and is 212,928 bytes in size.

### BACKGROUND

Genetic variants that influence complex traits are thought to regulate genes that work together in particular biological pathways. Identifying such convergence can help to discover genes and cellular functions that causally influence disease risk. However, it has been challenging to identify such convergence: complex traits often involve contributions from multiple cell types; most risk variants are noncoding and can regulate multiple nearby genes; and it remains unclear which genes work together in which pathways in which cell types.

Vascular diseases, such as coronary artery disease (CAD) continue to affect millions of people globally. In 2015, CAD affected 110 million people and resulted in 8.9 million deaths. CAD is a cause of 15.6% of all deaths world-wide, making it the most common cause of death globally.

Genome-wide Association Studies (GWAS) for CAD have discovered 306 independent signals. CAD heritability is significantly enriched in multiple cell types, including endothelial cells and vascular smooth muscle cells in the vessel wall, and hepatocytes, which influence cholesterol metabolism. At a few individual loci, noncoding risk variants have been shown to regulate the expression of key endothelial cell genes such as endothelial nitric oxide synthase (NOS3), endothelin 1 (EDN1), and others.

### SUMMARY

In one aspect, the present disclosure provides an engineered, non-naturally occurring gene editing system comprising (a) a single guide RNA (sgRNA) which comprises a guide sequence capable of hybridizing with a target sequence, or a polynucleotide encoding the sgRNA, and (b) an effector protein, or one or more nucleotide sequences encoding the effector protein; wherein the sgRNA hybridizes to said target sequence, and the sgRNA forms a complex with the effector protein; wherein the effector protein comprises a nuclease and/or an effector domain, wherein the sgRNA is capable of hybridizing to one or more target genes, wherein the one or more target gene is a gene of the Cerebral Cavernous Malformation (CCM) pathway or a gene that regulates the CCM pathway. In some embodiments, the one or more target genes are selected from the group consisting of: TLNRD1, CCM2, HEG1, ITGB1BP1, KRIT1, PDCD10, ARPC2, CDC42, CDH5, DNM2, MEAF6, PDCD7, RHOA, KLF2, KLF4, MAP2K5, MAP3K3, MEF2A, and NFAT5. In some embodiments, the sgRNA comprises a nucleotide sequence as set forth in any one of SEQ ID NO: 3-209. In some embodiments, the sgRNA comprises a nucleotide sequence as set forth in any one of SEQ ID NO: 4, 14, 48, 49, 84, 91, 93, 94, 129, 139, 200, 201, 202, 203, 204, 205, 206, 207, 208, and 209. In some embodiments, the effector protein comprises a zinc finger nuclease, a TALEN, or a Cas protein. In some embodiments, the gene editing system comprises a Clustered Regularly Interspaced Short Palindromic Repeats (CRISPR)-associated (CRISPR-Cas) system. In some embodiments, the effector protein comprises a Cas protein fused to a repression domain. In some embodiments, the repression domain is selected from the group consisting of KRAB, DNMT1, and HDAC.

In one aspect, the present disclosure provides a single guide RNA (sgRNA) comprising a nucleotide sequence as set forth in any one of SEQ ID NO: 3-209. In some embodiments, the sgRNA comprises a nucleotide sequence as set forth in any one of SEQ ID NO: 4, 14, 48, 49, 84, 91, 93, 94, 129, 139, 200, 201, 202, 203, 204, 205, 206, 207, 208, and 209.

In one aspect, the present disclosure provides an adeno-associated virus (AAV) particle comprising a gene editing system, such as any of the gene editing systems described above, or any of the sgRNAs described above.

In one aspect, the present disclosure provides a vector system comprising one or more vectors, wherein the one or more vectors comprises: (a) a first expression regulatory element operably linked to a nucleotide sequence encoding an effector protein, or one or more nucleotide sequences encoding the effector protein; and (b) a second expression regulatory element operably linked to one or more nucleotide sequences encoding a single guide RNA (sgRNA) comprising a guide sequence capable of hybridizing to a target sequence, wherein components (a) and (b) are located on the same or different vectors, wherein the sgRNA is capable of hybridizing to one or more target genes, wherein the one or more target gene is a gene of the Cerebral Cavernous Malformation (CCM) pathway or a gene that regulates the CCM pathway. In some embodiments, the one or more target genes are selected from the group consisting of: TLNRD1, CCM2, HEG1, ITGB1BP1, KRIT1, PDCD10, ARPC2, CDC42, CDH5, DNM2, MEAF6, PDCD7, RHOA, KLF2, KLF4, MAP2K5, MAP3K3, MEF2A, and NFAT5.

In one aspect, the present disclosure provides an isolated cell comprising any of the gene editing systems described above. In one aspect, the present disclosure provides an in vitro or ex vivo host cell or cell line or progeny thereof comprising any of the gene editing systems described above.

In one aspect, the present disclosure provides a method for treating a vascular disease in a subject comprising administering to the subject a therapeutically effective amount of a pharmacological agent capable of modulating the expression of a target gene in vascular endothelial cells, wherein the target gene is a Cerebral Cavernous Malformation (CCM) pathway gene or a gene that regulates the function of the CCM pathway. In some embodiments, modulating the expression of a target gene comprises reducing the expression of the target gene. In some embodiments, the vascular disease is coronary artery disease (CAD). In some embodiments, the subject has, is suspected of having, or is at risk for developing the vascular disease. In some embodiments, the target gene is selected from the group consisting of TLNRD1, CCM2, HEG1, ITGB1BP1, KRIT1, PDCD10, ARPC2, CDC42, CDH5, DNM2, MEAF6, PDCD7, RHOA, KLF2, KLF4, MAP2K5, MAP3K3, MEF2A, and NFAT5. In some embodiments, the pharmacological agent is a gene editing system. In some embodiments, the gene editing system comprises a Clustered Regularly Interspaced Short Palindromic Repeats (CRISPR)-associated (CRISPR-Cas) system, comprising (a) a single guide RNA (sgRNA) which comprises a guide sequence capable of hybridizing with the target sequence, or a polynucleotide encoding the sgRNA, and (b) an effector protein, or one or more nucleotide sequences encoding the effector protein; wherein the sgRNA hybridizes to the target sequence, and the sgRNA forms a complex with the effector protein; wherein the effector protein comprises a nuclease and/or an effector domain; wherein the sgRNA is capable of hybridizing to one or more genes selected from the group consisting of: TLNRD1, CCM2, HEG1, ITGB1BP1, KRIT1, PDCD10, ARPC2, CDC42, CDH5, DNM2, MEAF6, PDCD7, RHOA, KLF2, KLF4, MAP2K5, MAP3K3, MEF2A, and NFAT5. In some embodiments, the sgRNA comprises a nucleotide sequence as set forth in any one of SEQ ID NO: 3-209. In some embodiments, the sgRNA comprises a nucleotide sequence as set forth in any one of SEQ ID NO: 4, 14, 48, 49, 84, 91, 93, 94, 129, 139, 200, 201, 202, 203, 204, 205, 206, 207, 208, and 209. In some embodiments, the method further comprises administering to the subject a therapeutically effective amount of a pharmacological agent capable of increasing the activity of MEKK3, MEK5, ERK5, KLF2, or KLF4 in vascular endothelial cells of the subject. In some embodiments, the vascular endothelial cells are arterial endothelial cells. In some embodiments, the gene editing system specifically targets arterial endothelial cells.

In one aspect, the present disclosure provides a method for determining whether a subject having, suspected of having, or at risk for a vascular disease is likely to respond to a therapy for the vascular disease, comprising: (a) analyzing a biological sample obtained from the subject, wherein the biological sample comprises nucleic acids; (b) detecting the presence or absence of one or more nucleic acid sequence variant that results in loss-of-function of one or more genes of the Cerebral Cavernous Malformation (CCM) pathway or one or more genes that regulate the CCM pathway; and (c) determining that the subject is more likely to respond to the therapy if the sequence variant is detected. In some embodiments, the therapy comprises administering to the subject a therapeutically effective amount of a pharmacological agent capable of reducing the expression of a target gene in vascular endothelial cells, wherein the target gene is a Cerebral Cavernous Malformation (CCM) pathway gene or a gene that regulates the function of the CCM pathway. In some embodiments, the target gene is selected from the group consisting of TLNRD1, CCM2, HEG1, ITGB1BP1, KRIT1, PDCD10, ARPC2, CDC42, CDH5, DNM2, MEAF6, PDCD7, RHOA, KLF2, KLF4, MAP2K5, MAP3K3, MEF2A, and NFAT5. In some embodiments, the therapy comprises a CRISPR therapy. In some embodiments, the pharmacological agent is a gene editing system. In some embodiments, the gene editing system comprises a Clustered Regularly Interspaced Short Palindromic Repeats (CRISPR)-associated (CRISPR-Cas) system, comprising (a) a single guide RNA (sgRNA) which comprises a guide sequence capable of hybridizing with the target sequence, or a polynucleotide encoding the sgRNA, and (b) an effector protein, or one or more nucleotide sequences encoding the effector protein; wherein the sgRNA hybridizes to the target sequence, and the sgRNA forms a complex with the effector protein; wherein the effector protein comprises a nuclease and/or an effector domain; wherein the sgRNA is capable of hybridizing to one or more genes selected from the group consisting of: TLNRD1, CCM2, HEG1, ITGB1BP1, KRIT1, PDCD10, ARPC2, CDC42, CDH5, DNM2, MEAF6, PDCD7, RHOA, KLF2, KLF4, MAP2K5, MAP3K3, MEF2A, and NFAT5. In some embodiments, the sgRNA comprises a nucleotide sequence as set forth in any one of SEQ ID NO: 3-209. In some embodiments, the sgRNA comprises a nucleotide sequence as set forth in any one of SEQ ID NO: 4, 14, 48, 49, 84, 91, 93, 94, 129, 139, 200, 201, 202, 203, 204, 205, 206, 207, 208, and 209. In some embodiments, the vascular endothelial cells are arterial endothelial cells. In some embodiments, the gene editing system specifically targets arterial endothelial cells. In some embodiments, the method further comprises (d) determining that the subject is less likely to respond to the therapy if the sequence variant is not detected.

In one aspect, the present disclosure provides a method for modifying a target locus of interest, the method comprising delivering to the locus any of the gene editing systems described above, wherein the effector protein forms a complex with the sgRNA and upon binding of the complex to a target locus of interest, the effector protein induces a modification of the target locus of interest, wherein the target locus of interest is within a cell. In some embodiments, the cell is a vascular endothelial cell. In some embodiments, the cell is an arterial endothelial cell. In some embodiments, the effector protein is a Cas9 protein. In some embodiments, the target locus of interest comprises one or more genes selected from the group consisting of: TLNRD1, CCM2, HEG1, ITGB1BP1, KRIT1, PDCD10, ARPC2, CDC42, CDH5, DNM2, MEAF6, PDCD7, RHOA, KLF2, KLF4, MAP2K5, MAP3K3, MEF2A, and NFAT5. In some embodiments, the modification of the target locus of interest results in reduced expression of the target locus of interest in the cell. In some embodiments, the sgRNA comprises a nucleotide sequence as set forth in any one of SEQ ID NO: 3-209. In some embodiments, the sgRNA comprises a nucleotide sequence as set forth in any one of SEQ ID NO: 4, 14, 48, 49, 84, 91, 93, 94, 129, 139, 200, 201, 202, 203, 204, 205, 206, 207, 208, and 209.

In one aspect, the present disclosure provides a kit for determining whether a subject having, suspected of having, or at risk for a vascular disease is likely to respond to a therapy for the vascular disease comprising: (i) at least one PCR primer pair for PCR amplification of a CCM pathway gene or at least one probe for hybridizing to a CCM pathway gene under stringent hybridization conditions; and (ii) at least one PCR primer pair for PCR amplification of at least one housekeeping gene. In some embodiments, the kit further comprises instructions for using the kit. In some embodiments, the CCM pathway gene is selected from the group consisting of: TLNRD1, CCM2, HEG1, ITGB1BP1, KRIT1, PDCD10, ARPC2, CDC42, CDH5, DNM2, MEAF6, PDCD7, RHOA, KLF2, KLF4, MAP2K5, MAP3K3, MEF2A, and NFAT5. In some embodiments, at least one primer of a PCR primer pair for PCR amplification of a CCM2 gene hybridizes to a nucleic acid sequence encoding V74I of SEQ ID NO:1. In some embodiments, the at least one housekeeping gene is selected from the group consisting of GAPDH, ACTB, TUBB, UBQ, PGK, and RPL.

Both the foregoing summary and the following description of the drawings and detailed description are exemplary and explanatory. They are intended to provide further details of the disclosure, but are not to be construed as limiting. Other objects, advantages, and novel features will be readily apparent to those skilled in the art from the following detailed description of the disclosure.

It should be appreciated that all combinations of the foregoing concepts and additional concepts discussed in greater detail below are provided as being part of the inventive subject matter disclosed herein and may be employed in any combination to achieve the benefits described herein.

### BRIEF DESCRIPTION OF THE DRAWINGS

The patent or application file contains at least one drawing executed in color. Copies of this patent or patent application publication with color drawing(s) will be provided by the Office upon request and payment of necessary fee.

FIG. 1A shows an overview of Variant-to-Gene-to-Pathway (V2G2P) analysis. Red nodes outline an example of how risk variants could link to genes that converge onto specific Perturb-seq programs, identified by the V2G2P enrichment test, to provide new insights into mechanisms underlying disease risk.

FIG. 1B shows a diagram of an approach to create a map of gene programs and regulators using Perturb-seq (Gene to Program, G2P).

FIG. 1C shows a density plot of log 2 average knock down efficacy, across all 15 guides, for all targeted genes, or genes expressed at the indicated TPM thresholds. Average knock down was 40% for genes expressed at 300+ TPM (for which there was the greatest power to detect changes in expression).

FIG. 1D shows that fitness effects were estimated as the ratio of guide frequency in singlet cells to guide frequency in the original library. Guides to common essential genes (red) were depleted more frequently than guides to other genes.

FIG. 1E shows curated labels for all 50 programs that did not correlate with batch.

FIG. 1F shows profiles of Endothelial-Cell-Specific Programs. Organized by top 3 program co-regulated genes (left panel), transcription factor motifs in enhancers (by enrichment FDR, middle panel), and top 3 significant regulators (by fold-change in component expression, right panel).

FIG. 2A shows the path to the convergence of 5 V2G2P programs and 41 V2G2P genes for coronary artery disease. 1,942 expressed genes were identified in CAD loci, of which, 254 have a Variant-to-Gene (V2G) link, 883 contain a Gene-to-Program (G2P) link, and 127 have both V2G and G2P links. Through the V2G2P enrichment test, 5 V2G2P programs and 41 genes in V2G2P programs with V2G links (V2G2P genes) were prioritized.

FIG. 2B shows the identification of V2G2P for CAD. The 50 programs are ordered (y-axis) by the number of program genes linked to CAD variants (x-axis). The 5 programs with FDR<0.05 were defined as V2G2P programs. Gray dashed line: the number of genes linked to CAD variants that would be expected by chance.

FIG. 2C shows the relationships among the 41 V2G2P genes for CAD, and 5 V2G2P programs. Top: 6 V2G2P genes were regulators of one or more V2G2P programs (FDR<0.05). Light blue boxes indicate positive regulators (genes where loss-of-function leads to a decrease in program expression); and dark blue indicates negative regulators (genes where loss-of-function leads to an increase in program expression). Bottom: 36 V2G2P genes for CAD were co-regulated genes in one or more programs. Gray boxes indicate program membership. Cross hatching indicates genes that were previously known to affect CAD risk through effects in endothelial cells (Table 2).

FIG. 3A shows that genes that are members of the CCM complex and pathways regulate V2G2P programs for CAD. Color scale: average log₂ fold-change of effect of the perturbed gene on the 5 programs, with red shading indicating knock down leads to increased expression of Programs 8 and 48 and reduced expression of Programs 35, 39, and 47. Solid black lines indicate previously known physical or functional interactions. TLNRD1 is newly linked to the CCM complex via analysis. Dotted black lines indicate regulation of the 5 programs. Gray boxes indicate functionally related genes that were not tested in the Perturb-seq experiment. Bold text: V2G2P genes for CAD.

FIG. 3B shows the effects of genes in FIG. 3A on the 5 V2G2P programs. Color scale: log₂ fold-change on program expression in Perturb-seq.

FIG. 3C shows the effects of perturbing CCM pathway members on expression of 41 V2G2P genes for CAD. Color scale: log₂ fold-change on gene expression in individual knock down experiments assayed by bulk RNA-seq (average for two guides to each target). Bold genes in row names: V2G2P genes. Colored text in columns: Genes significantly regulated by one or more CCM pathway perturbation (FDR<0.05), red: upregulated by upstream signaling gene perturbations or downregulated by downstream gene perturbations, blue: vice versa.

FIG. 3D shows the likely direction of effect of V2G2P genes on atherosclerosis or vascular barrier dysfunction based on prior genetic studies in mouse models.

FIG. 4A shows 1,503 perturbed nearby genes to CAD GWAS loci, ordered by effect on the 5 V2G2P programs for CAD (average −log₁₀ p-value). Labels: top 5 genes. Red: V2G2P genes.

FIG. 4B shows 2,284 perturbed genes ordered by their similarity with CCM2 perturbation (correlation in log 2 effects on Program expression). Labels: as in FIG. 4A, for top and bottom 5 perturbed genes.

FIG. 4C shows 15q25.1 CAD risk locus, where rs1879454 is predicted to regulate TLNRD1 (red arc). GWAS variants: −log₁₀ GWAS P-value for variants with R²>0.9 with the lead SNP. Green signal tracks: Epigenomic data from endothelial cells. Gray signal tracks: data from other cell types. HUVEC: human umbilical vein endothelial cells. CaSMCs: coronary artery smooth muscle cells. Red arc indicates that the enhancer containing rs1879454 regulates TLNRD1 expression (see FIGS. 4D and 4F).

FIG. 4D shows CRISPRi-FlowFISH targeting chromatin accessible elements around TLNRD1, including the candidate enhancer overlapping rs1879454. Each point represents the average effect on TLNRD1 gene expression of a single gRNA across 4 replicate FlowFISH experiments. Gray and red bars: elements in which CRISPRi leads to either no significant change (gray) or a significant decrease (red) in expression. Red numbers indicate FDR (−log 10).

FIG. 4E shows a graph illustrating that the enhancer containing rs1879454 regulates TLNRD1 in TeloHAEC, as measured by CRISPRi-FlowFISH. Bar and whiskers show mean±s.e.m. Dots show effects of individual gRNAs on expression for 117 negative control gRNAs (Control), 37 gRNAs targeting the promoter of TLNRD1, and 17 gRNAs targeting the enhancer containing rs1879454, averaged per guide across 4 CRISPRi-FlowFISH replicates.

FIG. 4F shows a zoom-in on the enhancer containing rs1879454. Colored bar in signal tracks indicates read coverage of the reference (C, blue) and alternate (A, green) alleles. Bottom shows the position-weight matrix for a composite GATA/TAL motif and the genome sequence with reference and alternate alleles highlighted in gray. The allele-specificity of chromatin accessibility, measured by DNAse, was also examined in HMVEC (human microvascular endothelial cells, 1.9-fold decrease, p-value=0.0192). FIG. 4F discloses SEQ ID NO: 236.

FIG. 4G shows allele-specific counts for rs1879454 in ATAC-seq in TeloHAEC, DNase-seq in HMVEC, and GATA-2 ChIP-seq in HUVEC. Reads were re-aligned to both reference and alternate alleles to avoid bias toward the reference allele.

FIG. 5A shows an AlphaFold2.3 Multimer model for TLNRD1, CCM2, PDCD10 and KRIT1. Right: predicted interaction between TLNRD1 (residues P177-V237) and the C-terminal helix of CCM2 (residues E417-S443). Left: Recapitulation of the known CCM2/KRIT1 binding site in the PTB (phosphotyrosine binding) domain of CCM2 with KRIT1 residues D225-N331. HHD: harmonin homology domain. Dashed green lines represent flexible loops. Amino acid positions are given to indicate the boundaries of predicted alpha-helix and beta-sheet structural features

FIG. 5B shows an immunoblot of FLAG-tagged TLNRD1 and/or V5-tagged CCM2 full length (“WT”) or C-terminal truncation (“4”) in an experiment in which said proteins were expressed in HEK293T cells, as indicated. Extracts were co-immunoprecipitated with mouse anti-FLAG and blotted with rabbit anti-V5 (top) or anti-TLNRD1 (bottom).

FIG. 5C is a heatmap of TLNRD1 and CCM2-regulated genes that affect CAD-relevant endothelial cell functions, in cells with the indicated knockdowns. Black text: the TLNRD1 and CCM2 knockdown targets. Green text: likely atheroprotective genes. Red text: likely atherogenic genes. Included are all genes that either 1) were in the top 40 TLNRD1 and CCM2 up- or down-regulated genes (by highest p.value across both knockdowns), had an average fold change in TLNRD1 and CCM2 knockdowns of >2, and had prior evidence for functions predicted to increase or decrease CAD-relevant endothelial cell phenotypes (noted above each gene name), and/or 2) were known EC-acting CAD genes that were significantly regulated by both TLNRD1 and CCM2 knockdowns (both p.<0.05).

FIG. 5D shows the trans-endothelial electrical resistance (TEER), at 4000 Hz frequency, was measured over 50 hrs, for CRISPRi teloHAEC cells expressing single guides targeting CCM2 or TLNRD1, or non-targeting controls. Thrombin was added 25 hrs after cell seeding, to disrupt cell junctions, and TEER measurements for each well normalized to the average value for the 4 hours before thrombin addition. Data for 2 different guides per target were averaged. N=6 to 8. Ranges: SEM.

FIG. 5E shows a boxplot of normalized TEER signal, from FIG. 5D, averaged for hours 45 to 50 (20-25 hrs post-thrombin). Center line, median; box limits, upper and lower quartiles; whiskers, 1.5× interquartile range; points, outliers. Significance was assessed by two-sided T-test.

FIG. 5F shows representative images of CRISPRi teloHAEC with control non-targeting guides or with guides to CCM2 or TLNRD1, that were subjected to flow in an Ibidi flow chamber for 48 hours. Cells were imaged by phase contrast microscopy using a 20× objective.

FIG. 5G shows the normal alignment to flow in control teloHAEC (measured as the angle, relative to flow, of the long axis of each cell) is significantly abrogated in both CCM2 & TLNRD1 KD cells (increased average angle relative to flow). Average values for all cells in each of 4 images for each of 2 guides per target were calculated (35 to 103 cells per image). Significance was assessed by T-test on these average values. Bars: SEM. Note that alignment to flow is not completely blocked in CCM2 or TLNRD1 KD cells, since the average angle relative to flow does not reach the 45% value expected if orientation were entirely random.

FIG. 5H shows the data from FIG. 5G, but measuring the ratio of the long vs. short axis lengths for each cell (“length/width”) from the fit ellipse function in FiJi.

FIG. 5I shows CRISPRi teloHAEC with control, TLNRD1 or CCM2 guides treated with doxycycline for 5 days, then fixed and stained with phalloidin to measure filamentous actin, and 63× confocal images collected (1 well each for 2 separate guides to each target, 25 images per well). Representative maximum-projection images.

FIG. 5J shows a quantitation of actin fiber characteristics in the phalloidin channel from maximum-projection images was performed as per. For fiber intensity per cell area, integrated intensity in skeletonized fibers was divided by cell area. Boxplots (inset in violin plots), as per FIG. 5E. N for cells with control, CCM2 or TLNRD1 guides was 145, 47, and 117, respectively.

FIG. 5K shows the data from FIG. 5J, but showing the number of fibers per cell.

FIG. 5L shows the data from FIG. 5J, but showing parallelness of actin fibers. A score of 0 indicates randomly oriented fibers, and a score of 1 indicates all fibers in a cell are parallel to each other.

FIG. 5M shows atrial enlargement and atrioventricular valve (AV) dilation in ccm2 and tlnrd1 knockdowns. Top: Representative confocal microscopic images showing a merged light microscopic and fluorescent (cardiac myosin light chain 2/cmlc2-GFP in cardiomyocytes) image of 50 hour post-fertilization zebrafish embryos (anterior to the left). Bottom: 3× zoomed-in fluorescent-only image of the heart (yellow boxes, above). N=5 embryos were analyzed per group. a: atrium, v: ventricle, av: atrioventricular valve.

FIG. 5N shows qRT-PCR results for knockdown of tlnrd1 & induction of klf2b in zebrafish embryos treated with CRISPR guides to tlnrd1, or with control tracrRNA. Signal was normalized to Actin, and then to the average for controls. n=9 for klf2b (6 for guide AF, 3 for guide AN.2). n=5 for tlnrd1 (2 for guide AF, 3 for guide AN.2). Bars: SEM. Significance was assessed by two-sided T-test.

FIG. 5O shows qRT-PCR results for knockdown of TLNRD1 & induction of KLF2 in TeloHAEC with Cas9-guide nucleofection knock down of TLNRD1 (or non-targeting guides, “Control”). Signal was normalized to GAPDH, and then to the average for controls. n=4 separate samples. Quantitation as in FIG. 5N.

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular, exemplary embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. Although not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety. In case of conflict, the present specification, including definitions, will control. Other aspects are set forth within the claims that follow.

The practice of the present technology will employ, unless otherwise indicated, conventional techniques of tissue culture, immunology, molecular biology, microbiology, chemical engineering, and cell biology, which are within the skill of the art.

Unless the context indicates otherwise, it is specifically intended that the various features described herein can be used in any combination. Moreover, the disclosure also contemplates that in some embodiments, any feature or combination of features set forth herein can be excluded or omitted. To illustrate, if the specification states that a complex comprises components A, B, and C (or A, B, and/or C), it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations that can be varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”.

### I. Introduction

The present disclosure relates to the discovery of novel genes, gene variants, and gene programs involved in the etiology of vascular diseases, such as coronary artery disease (CAD). This disclosure also provides methods of treating vascular diseases (e.g., CAD), methods of determining the likelihood that a subject will respond to a particular therapy for a vascular disease, and kits for the same.

Genome-wide association studies (GWAS) have led to the discovery of thousands of risk loci for common, complex diseases, each of which could point to genes and gene programs that influence disease. For some diseases, it has been observed that GWAS signals converge on a smaller number of biological pathways, and that this convergence can help to identify causal genes. However, identifying such convergence has been challenging: each GWAS locus can have many candidate genes, each gene might act in one or more possible pathways, and it can be unclear which programs might influence disease risk.

To address these challenges, an ideal approach would be to build comprehensive maps of enhancers and gene pathways in a given cell type in an unbiased way, such that one could link GWAS variants to the genes they regulate, link genes to the programs they regulate, and determine which of those programs might be relevant to disease risk. As detailed in the non-limiting Examples presented herein, the present disclosure addresses these challenges.

Described herein are methods that generate and employ unbiased maps to link disease variants to genes to programs (V2G2P) in a given cell type. These approaches were applied to study the role of genetics in pathological changes of endothelial cells associated with a particular disease, such as coronary artery disease (CAD). While coronary artery disease (CAD) is the disease assessed in the non-limiting Examples provided herein, the provided methods could be used for any disease. Briefly, to link variants to genes, enhancer-gene maps were constructed using the Activity-by-Contact model. To link genes to programs, CRISPRi-Perturb-seq was used to knock down all expressed genes within 500 Kb of CAD GWAS signals, and the effect of such knock down on gene expression programs was assessed using single-cell RNA-seq. By combining these variant-to-gene and gene-to-program maps, it was revealed that 43 of 306 CAD GWAS signals converge onto 5 gene programs linked to the cerebral cavernous malformations (CCM) pathway-which is known to coordinate transcriptional responses in endothelial cells but has not been previously linked to CAD risk. An exemplary regulator of these programs is TLNRD1, which the experiments described herein show is a new CAD gene and novel regulator of the CCM pathway. TLNRD1 loss-of-function alters actin organization and barrier function in endothelial cells in vitro, and heart development in zebrafish in vivo.

Taken together, the studies described herein have identified convergence of CAD risk loci into prioritized gene programs in endothelial cells, nominated new genes of potential therapeutic relevance for CAD, and demonstrated a generalizable strategy to connect disease variants to functions. These new insights are useful for the treatment of vascular diseases, such as CAD, as described in greater detail below.

### II. Definitions

As used in the description of the invention and the appended claims, the singular forms “a,” “an” and “the” are intended to include the plural forms as well, unless the context clearly indicates otherwise.

The terms “substantially” and “about” are used herein to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. When referring to a first numerical value as “substantially” or “about” the same as a second numerical value, the terms can refer to the first numerical value being within a range of variation of less than or equal to ±10% of the second numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. The terms or “acceptable,” “effective,” or “sufficient” when used to describe the selection of any components, ranges, dose forms, etc. disclosed herein intend that said component, range, dose form, etc. is suitable for the disclosed purpose.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

Also as used herein, “and/or” refers to and encompasses any and all possible combinations of one or more of the associated listed items, as well as the lack of combinations when interpreted in the alternative (“or”).

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Examples and implementations defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, “optional” or “optionally” means that the subsequently described event or circumstance can or cannot occur, and that the description includes instances where the event or circumstance occurs and instances where it does not.

A primer pair that specifically hybridizes under stringent conditions to a target nucleic acid may hybridize to any portion of the gene. As a result, the entire gene may be amplified or a segment of the gene may be amplified, depending on the portion of the gene to which the primers hybridize.

The terms “amplification” or “amplify” as used herein include methods for copying a target nucleic acid, thereby increasing the number of copies of a selected nucleic acid sequence. Amplification may be exponential or linear. A target nucleic acid may be DNA (such as, for example, genomic DNA and cDNA) or RNA. The sequences amplified in this manner form an “amplicon.” While the exemplary methods described hereinafter relate to amplification using the polymerase chain reaction (PCR), numerous other methods are known in the art for amplification of nucleic acids (e.g., isothermal methods, rolling circle methods, etc.). The skilled artisan will understand that these other methods may be used either in place of, or together with, PCR methods. See, e.g., Saiki, “Amplification of Genomic DNA” in PCR Protocols, Innis et al., Eds., Academic Press, San Diego, CA 1990, pp 13-20; Wharam, et al., Nucleic Acids Res. 2001 Jun. 1; 29(11):E54-E54; Hafner, et al., Biotechniques 2001 April; 30(4):852-860.

The terms “complement,” “complementary,” or “complementarity” as used herein with reference to polynucleotides (i.e., a sequence of nucleotides such as an oligonucleotide or a target nucleic acid) refer to standard Watson/Crick pairing rules. The complement of a nucleic acid sequence such that the 5′ end of one sequence is paired with the 3′ end of the other, is in “antiparallel association.” For example, the sequence “5′-A-G-T-3” is complementary to the sequence “3′-T-C-A-5′.” Certain bases not commonly found in natural nucleic acids may be included in the nucleic acids described herein; these include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be a sequence of RNA complementary to the DNA sequence or its complement sequence, and can also be a cDNA. The term “substantially complementary” as used herein means that two sequences specifically hybridize (defined below). The skilled artisan will understand that substantially complementary sequences need not hybridize along their entire length. A nucleic acid that is the “full complement” or that is “fully complementary” to a reference sequence consists of a nucleotide sequence that is 100% complementary (under Watson/Crick pairing rules) to the reference sequence along the entire length of the nucleic acid that is the full complement. A full complement contains no mismatches to the reference sequence.

A “fragment” in the context of a nucleic acid refers to a sequence of nucleotide residues which are at least about 5 nucleotides, at least about 7 nucleotides, at least about 9 nucleotides, at least about 11 nucleotides, or at least about 17 nucleotides. The fragment is typically less than about 300 nucleotides, less than about 100 nucleotides, less than about 75 nucleotides, less than about 50 nucleotides, or less than 30 nucleotides. In certain embodiments, the fragments can be used in polymerase chain reaction (PCR), various hybridization procedures or microarray procedures to identify or amplify identical or related parts of mRNA or DNA molecules. A fragment or segment may uniquely identify each polynucleotide sequence of the disclosure.

“Genomic nucleic acid” or “genomic DNA” refers to some or all of the DNA from a chromosome. Genomic DNA may be intact or fragmented (e.g., digested with restriction endonucleases by methods known in the art). In some embodiments, genomic DNA may include sequence from all or a portion of a single gene or from multiple genes. In contrast, the term “total genomic nucleic acid” is used herein to refer to the full complement of DNA contained in the genome. Methods of purifying DNA and/or RNA from a variety of samples are well-known in the art.

As used herein, the term “oligonucleotide” refers to a short polymer composed of deoxyribonucleotides, ribonucleotides or any combination thereof. Oligonucleotides are generally at least about 10, 11, 12, 13, 14, 15, 20, 25, 40 or 50 up to about 100, 110, 150 or 200 nucleotides (nt) in length, such as from about 10, 11, 12, 13, 14, or 15 up to about 70 or 85 nt, such as from about 18 up to about 26 nt in length. The single letter code for nucleotides is as described in the U.S. Patent Office Manual of Patent Examining Procedure, section 2422, table 1. In this regard, the nucleotide designation “R” means purine such as guanine or adenine, “Y” means pyrimidine such as cytosine or thymidine (uracil if RNA); and “M” means adenine or cytosine. An oligonucleotide may be used as a primer or as a probe.

As used herein, a “primer” for amplification is an oligonucleotide that is complementary to a target nucleotide sequence and leads to addition of nucleotides to the 3′ end of the primer in the presence of a DNA or RNA polymerase. The 3′ nucleotide of the primer should generally be identical to the target nucleic acid sequence at a corresponding nucleotide position for optimal expression and amplification. The term “primer” as used herein includes all forms of primers that may be synthesized including peptide nucleic acid primers, locked nucleic acid primers, phosphorothioate modified primers, labeled primers, and the like. As used herein, a “forward primer” is a primer that is complementary to the anti-sense strand of dsDNA. A “reverse primer” is complementary to the sense-strand of dsDNA. An “exogenous primer” refers specifically to an oligonucleotide that is added to a reaction vessel containing the sample nucleic acid to be amplified from outside the vessel and is not produced from amplification in the reaction vessel. A primer that is “associated with” a fluorophore or other label is connected to the label through some means. An example is a primer-probe.

Primers are typically from at least 10, 15, 18, or 30 nucleotides in length up to about 100, 110, 125, or 200 nucleotides in length, such as from at least 15 up to about 60 nucleotides in length, and such as from at least 25 up to about 40 nucleotides in length. In some embodiments, primers and/or probes are 15 to 35 nucleotides in length. There is no standard length for optimal hybridization or polymerase chain reaction amplification. An optimal length for a particular primer application may be readily determined in the manner described in H. Erlich, PCR Technology, Principles and Application for DNA Amplification, (1989).

A “primer pair” is a pair of primers that are both directed to target nucleic acid sequence. A primer pair contains a forward primer and a reverse primer, each of which hybridizes under stringent condition to a different strand of a double-stranded target nucleic acid sequence. The forward primer is complementary to the anti-sense strand of the dsDNA and the reverse primer is complementary to the sense-strand. One primer of a primer pair may be a primer-probe (i.e., a bi-functional molecule that contains a PCR primer element covalently linked by a polymerase-blocking group to a probe element and, in addition, may contain a fluorophore that interacts with a quencher).

An oligonucleotide (e.g., a probe or a primer) that is specific for a target nucleic acid will “hybridize” to the target nucleic acid under specified conditions. As used herein, “hybridization” or “hybridizing” refers to the process by which an oligonucleotide single strand anneals with a complementary strand through base pairing under defined hybridization conditions.

“Specific hybridization” is an indication that two nucleic acid sequences share a high degree of complementarity. Specific hybridization complexes form under permissive annealing conditions and remain hybridized after any subsequent washing steps. Permissive conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to be about 5° C. to 20° C. lower than the thermal melting point (Tm) for the specific sequence at a defined ionic strength and pH. The Tm is the temperature (under defined ionic strength and pH) at which 50% of the target nucleic acid hybridizes to a perfectly matched probe. Equations for calculating Tm and conditions for nucleic acid hybridization are known in the art. Specific hybridization may occur under stringent conditions, which are well known in the art. Stringent hybridization conditions are hybridization in 50% formamide, 1 M NaCl, 1% SDS at 37° C., and a wash in 0.1×SSC at 60° C. Hybridization procedures are well known in the art and are described in e.g. Ausubel et al, Current Protocols in Molecular Biology, John Wiley & Sons Inc., 1994.

As used herein, an oligonucleotide is “specific” for a nucleic acid if the oligonucleotide has at least 50% sequence identity with the nucleic acid when the oligonucleotide and the nucleic acid are aligned. An oligonucleotide that is specific for a nucleic acid is one that, under the appropriate hybridization or washing conditions, is capable of hybridizing to the target of interest and not substantially hybridizing to nucleic acids which are not of interest. In some embodiments, higher levels of sequence identity include at least 75%, at least 80%, at least 85%, at least 90%, at least 95% and at least 98% sequence identity. Sequence identity can be determined using a commercially available computer program with a default setting that employs algorithms well known in the art. As used herein, sequences that have “high sequence identity” have identical nucleotides at least at about 50% of aligned nucleotide positions, such as at least at about 60% of aligned nucleotide positions, such as at least at about 75% of aligned nucleotide positions.

Oligonucleotides used as primers or probes for specifically amplifying (i.e., amplifying a particular target nucleic acid) or specifically detecting (i.e., detecting a particular target nucleic acid sequence) a target nucleic acid generally are capable of specifically hybridizing to the target nucleic acid under stringent conditions.

As used herein, the term “sample” or “test sample” may comprise clinical samples, isolated nucleic acids, or isolated microorganisms. In some embodiments, a sample is obtained from a biological source (i.e., a “biological sample”), such as tissue, bodily fluid, or microorganisms collected from a subject. Sample sources include, but are not limited to, sputum (processed or unprocessed), bronchial alveolar lavage (BAL), bronchial wash (BW), blood, bodily fluids, cerebrospinal fluid (CSF), urine, plasma, serum, or tissue (e.g., biopsy material). Exemplary sample sources include nasopharyngeal swabs, wound swabs, and nasal washes. The term “patient sample” as used herein refers to a sample obtained from a human seeking diagnosis and/or treatment of a disease.

As used herein, the term “polymorphism” refers to the existence of two or more different nucleotide sequences at a particular locus in the DNA of the genome. Polymorphisms can serve as genetic markers and may also be referred to as genetic variants. Polymorphisms include nucleotide substitutions, insertions, deletions and microsatellites, and may, but need not, result in detectable differences in gene expression or protein function. A polymorphic site is a nucleotide position within a locus at which the nucleotide sequence varies from a reference sequence in at least one individual in a population.

A “variant” or “genetic variant” as used herein, refers to a specific isoform of a haplotype found in a population, the specific form differing from other forms of the same haplotype in at least one, and frequently more than one, variant sites or nucleotides within the region of interest in the gene. The sequences at these variant sites that differ between different alleles of a gene are termed “gene sequence variants,” “alleles,” or “variants.” The term “alternative form” refers to an allele that can be distinguished from other alleles by having at least one, and frequently more than one, variant sites within the gene sequence. “Variants” include isoforms having single nucleotide polymorphisms (SNPs) and deletion/insertion polymorphisms (DIPs). Reference to the presence of a variant means a particular variant, i.e., particular nucleotides at particular polymorphic sites, rather than just the presence of any variance in the gene.

The term “genotype”, as used herein, refers to the particular allelic form of a gene, which can be defined by the particular nucleotide(s) present in a nucleic acid sequence at a particular site(s). Genotype may also indicate the pair of alleles present at one or more polymorphic loci. For diploid organisms, such as humans, two haplotypes make up a genotype. Genotyping is any process for determining a genotype of an individual, e.g., by nucleic acid amplification, DNA sequencing, antibody binding, or other chemical analysis (e.g., to determine the length). The resulting genotype may be unphased, meaning that the sequences found are not known to be derived from one parental chromosome or the other.

“Treat,” “treating,” or “treatment” as used herein refers to any type of measure that imparts a benefit to a patient afflicted with or at risk for developing a disease, including characterizing a condition of a patient, diagnosing a condition of the patient, monitoring a condition of the patient, prognosing a condition of the patient, improvement in the condition of the patient (e.g., in one or more symptoms), delay in the onset or progression of the disease, prevention of disease, etc. Treatment may include any drug, drug product, method, procedure, lifestyle change, or other adjustment introduced in an attempt to diagnose, characterize, monitor, prognose, and/or change a particular aspect of a subject's health (i.e., directed to a particular disease, disorder, or condition).

The term “pharmacological agent” or “therapeutic agent” as used herein refers to any composition that imparts a benefit to a subject or patient afflicted with or at risk for developing a disease, including improvement in the condition of the subject or patient (e.g., in one or more symptoms), delay in the onset or progression of the disease, prevention of disease, treatment of the disease, etc. A pharmacological agent or therapeutic agent may refer to a chemical compound, such as a drug, pro-drug, small-molecule drug, etc. A pharmacological agent or therapeutic agent may refer to a biological compound, such as a therapeutic nucleic acid, protein, peptide, polypeptide, protein complex, cell, cell extract, biological fluid, etc. A pharmacological agent or therapeutic agent can be or comprise a gene therapy. In some embodiments, a pharmacological agent includes a system for modulating the expression of one or more target genes. For example, a pharmacological agent can include a gene-editing or nuclease system, such as a Clustered Regularly Interspaced Short Palindromic Repeats (CRISPR)-Cas system.

As used herein, the term “modulation” or “modulating” in the context of gene expression or function refers to effecting a change in the activity of a gene or a gene product therefrom. Modulation of expression can include, but is not limited to, gene activation and gene repression. Modulation of expression of a gene can refer to reducing or increasing the expression of a gene. Genome editing (e.g., cleavage, alteration, inactivation, random mutation) can be used to modulate expression of a gene. Gene inactivation refers to any reduction in gene expression as compared to a cell that does not include a gene editing system as described herein. Thus, gene inactivation may be partial or complete.

The term “at risk”, as used in the context of a subject at risk for a particular disease or disorder (e.g., a vascular disease, e.g., CAD), refers to a likelihood that a subject will have or develop the particular disease. A subject may be at risk for a particular disease or disorder due to one or more factors. Factors may include but are not limited to genetic predispositions, age, height, weight, sex, race, nationality, ethnicity, sexual orientation, family health history, lifestyle and behavioral factors (such as diet, exercise, alcohol consumption, etc.), and clinical risk factors (e.g., other diseases or disorders). In some cases, a subject at risk for a particular disease is a subject who does not have or who has not yet developed the particular disease. Notable risk factors for vascular diseases, such as CAD, include but are not limited to lifestyle and behavioral factors such as a high-sugar or high-fat diet, low exercise, and alcohol consumption, and clinical factors such as pre-existing atrial fibrillation, hypertension, and obesity.

As used herein, the term “detecting” refers to observing a signal from a detectable label to indicate the presence of a target. More specifically, detecting is used in the context of detecting a specific sequence of a target nucleic acid molecule. The term “detecting” used in context of detecting a signal from a detectable label to indicate the presence of a target nucleic acid in the sample does not require the method to provide 100% sensitivity and/or 100% specificity. In some embodiments, sensitivity is at least 50%, at least 60%, at least 70%, at least 80%, at least 90%, or at least 99%. In some embodiments, the specificity is at least 50%, at least 60%, at least 70%, at least 80%, at least 90%, or at least 99%. Detecting also encompasses assays that produce false positives and false negatives. False negative rates can be 1%, 5%, 10%, 15%, 20% or even higher. False positive rates can be 1%, 5%, 10%, 15%, 20% or even higher. As used herein, “detecting” may also refer to observing a signal indicating the presence and/or amount of a substance, such as a protein in a sample.

As used herein, the term “target gene” refers to a gene that is a member of the Cerebral Cavernous Malformation (CCM) pathway, or a gene that regulates the CCM pathway. The term “target gene” encompasses genes that encode the members of the CCM complex, such as KRIT1 (CCM1), CCM2, and PDCD10 (CCM3). The term “target gene” also encompasses genes that regulate the CCM pathway, including TLNRD1, HEG1, ITGB1BP1, ARPC2, CDC42, CDH5, DNM2, MEAF6, PDCD7, RHOA, KLF2, KLF4, MAP2K5, MAP3K3, MEF2A, and NFAT5. Accordingly, the term “target gene” encompasses at least the following genes: TLNRD1, CCM2, HEG1, ITGB1BP1, KRIT1, PDCD10, ARPC2, CDC42, CDH5, DNM2, MEAF6, PDCD7, RHOA, KLF2, KLF4, MAP2K5, MAP3K3, MEF2A, and NFAT5.

As used herein, the term “CAD-associated” gene refers to a gene that is linked to the etiology, diagnosis, or prognosis of CAD, for example, by a showing that modulation of expression or function of the gene (such as by the presence of a mutation resulting in loss-of-function of a gene product of the gene), positively or negatively impacts CAD risk. Non-limiting examples of CAD-associated genes include BCAR1, BMP1, CALCRL, CCM2, CDKN1A, CDKN2B, CFDP1, COL4A1, COL4A2, EXOC3L2, FBN2, FGD6, FLT1, FURIN, GDPD5, GGT5, GOSR2, IBTK, LAMB2, LOX, MORF4L1, N4BP2L2, NOS3, PALLD, PECAM1, PGF, PLPP3, PREX1, PRKAR1A, SCUBE1, SERPINH1, SH3PXD2A, SLK, SMAD3, SPRY4, SVIL, SWAP70, TFPI, TLNRD1, TSPAN14, and ZEB2.

One example of a gene that is a target gene is CCM2. As used herein, the term “CCM2 gene” refers to the CCM2 gene, which encodes the CCM2 protein. As used herein, the term can refer to any nucleic acid encoding a CCM2 protein, such as genomic DNA, mRNA, cDNA, or other engineered/recombinant nucleic acid, or portions thereof. The term encompasses the nucleic acid sequences set forth in NCBI Accession Numbers NM_001029835; NM_001167934; NM_001167935; NM_031443; NM_001363458; NM_001190343; NM_001190344; and NM_146014, or the coding region thereof, as well as natural and engineered isoforms and variants. The term includes RNA transcripts encoding all or a portion of SEQ ID NO: 1, genomic sequences encoding SEQ ID NO: 1, and all untranslated CCM2 genomic sequences, such as, for example, introns, untranslated leader regions, and polyadenylation signals. Illustrative nucleic acid sequences encompassed by the term are publicly available at National Center for Biotechnology Information, Bethesda, MD (www.ncbi.nlm.nih.gov) and HUGO Gene Nomenclature Committee, Cambridge, UK (www.genenames.org).

As used herein, the term “CCM2 protein” refers generally to the CCM2 protein, also known in the art as C7orf22; OSM; pp 10187; CCM2 scaffolding protein; and CCM2 scaffold protein. As used herein, the term can refer to any CCM2 protein, polypeptide, or a portion thereof. The term encompasses the amino acid sequences set forth in NCBI Accession Numbers NP_001025006; NP_001161406; NP_001161407; NP_113631; NP_001350387; NP 001350388; NP_001177272; NP_001177273; and NP_666126, as well as natural and engineered isoforms and variants. An amino acid sequence of human CCM2 protein is set forth in SEQ ID NO:1.

(SEQ ID NO: 1)
MHSSCRQRRNQNLSKEIPQTEFHTGYSMENEPGIVSPFKRVFLKGEKSR
DKKAHEKVTERRPLHTVVLSLPERVEPDRLLSDYIEKEVKYLGQLTSIP
GYLNPSSRTEILHFIDNAKRAHQLPGHLTQEHDAVLSLSAYNVKLAWRD
GEDIILRVPIHDIAAVSYVRDDAAHLVVLKTAQDPGISPSQSLCAESSR
GLSAGSLSESAVGPVEACCLVILAAESKVAAEELCCLLGQVFQVVYTES
TIDFLDRAIFDGASTPTHHLSLHSDDSSTKVDIKETYEVEASTFCFPES
VDVGGASPHSKTISESELSASATELLQDYMLTLRTKLSSQEIQQFAALL
HEYRNGASIHEFCINLRQLYGDSRKFLLLGLRPFIPEKDSQHFENFLET
IGVKDGRGIITDSFGRHRRALSTTSSSTTNGNRATGSSDDRSAPSEGDE
WDRMISDISSDIEALGCSMDQDSA

As used herein, the term “CCM2 mutant” or “CCM2 variant” refers generally to a CCM2 nucleic acid or an amino acid sequence that differs from the wild-type sequence of CCM2, such as that set forth, for example, in SEQ ID NO: 1. The term includes all manner of mutations known in the art, including, but not limited to, insertions, deletions, substitutions, and inversions, encompasses both silent mutations and those that alter CCM2 function, and encompasses gain-of-function and loss-of-function mutations. In some embodiments described herein, CCM2 mutations comprise a single nucleotide polymorphism (SNP). In some embodiments, the SNP is rs2107732, which is a single-nucleotide variation (SNV) of G to A, which results in a substitution of a Valine to an Isoleucine at amino acid position 74 of SEQ ID NO: 1. rs2107732 is located at position chr7:45038379 (GRCh38.p13). This change can be described as V74I, or Val74Ile.

CCM2 nucleic acid and protein sequences described herein can be isolated from any source, including, but not limited to, a human patient, a laboratory animal or veterinary animal (e.g., dog, pig, cow, horse, rat, mouse, etc.), a sample therefrom (e.g., tissue or body fluid, or extract thereof), or a cell therefrom (e.g., primary cell or cell line, or extract thereof).

Another example of a gene that is a target gene is TLNRD1. As used herein, the term “TLNRD1 gene” refers to the TLNRD1 gene, which encodes the TLNRD1 protein. As used herein, the term can refer to any nucleic acid encoding a TLNRD1 protein, such as genomic DNA, mRNA, cDNA, or other engineered/recombinant nucleic acid, or portions thereof. The term encompasses the nucleic acid sequences set forth in NCBI Accession Numbers NM 022566 and NM_030705, or the coding region thereof, as well as natural and engineered isoforms and variants. The term includes RNA transcripts encoding all or a portion of SEQ ID NO: 2, genomic sequences encoding SEQ ID NO: 2, and all untranslated TLNRD1 genomic sequences, such as, for example, introns, untranslated leader regions, and polyadenylation signals. Illustrative nucleic acid sequences encompassed by the term are publicly available at National Center for Biotechnology Information, Bethesda, MD (www.ncbi.nlm.nih.gov) and HUGO Gene Nomenclature Committee, Cambridge, UK (www.genenames.org).

As used herein, the term “TLNRD1 protein” refers generally to the TLNRD1 protein, also known in the art as MESDC1, mesoderm development candidate 1, and talin rod domain containing 1. As used herein, the term can refer to any TLNRD1 protein, polypeptide, or a portion thereof. The term encompasses the amino acid sequences set forth in NCBI Accession Numbers NP_072088 and NP_109630, as well as natural and engineered isoforms and variants. An amino acid sequence of human TLNRD1 protein is set forth in SEQ ID NO: 2.

(SEQ ID NO: 2)
MASGSAGKPTGEAASPAPASAIGGASSQPRKRLVSVCDHCKGKMQLVAD
LLLLSSEARPVLFEGPASSGAGAESFEQCRDTIIARTKGLSILTHDVQS
QLNMGRFGEAGDSLVELGDLVVSLTECSAHAAYLAAVATPGAQPAQPGL
VDRYRVTRCRHEVEQGCAVLRATPLADMTPQLLLEVSQGLSRNLKFLTD
ACALASDKSRDRFSREQFKLGVKCMSTSASALLACVREVKVAPSELARS
RCALFSGPLVQAVSALVGFATEPQFLGRAAAVSAEGKAVQTAILGGAMS
VVSACVLLTQCLRDLAQHPDGGAKMSDHRERLRNSACAVSEGCTLLSQA
LRERSSPRTLPPVNSNSVN

As used herein, the term “TLNRD1 mutant” or “TLNRD1 variant” refers generally to a TLNRD1 nucleic acid or an amino acid sequence that differs from the wild-type sequence of TLNRD1, such as that set forth, for example, in SEQ ID NO: 2. The term includes all manner of mutations known in the art, including, but not limited to, insertions, deletions, substitutions, and inversions, encompasses both silent mutations and those that alter TLNRD1 function, and encompasses gain-of-function and loss-of-function mutations. In some embodiments described herein, TLNRD1 mutations comprise a single nucleotide polymorphism (SNP).

TLNRD1 nucleic acid and protein sequences described herein can be isolated from any source, including, but not limited to, a human patient, a laboratory animal or veterinary animal (e.g., dog, pig, cow, horse, rat, mouse, etc.), a sample therefrom (e.g., tissue or body fluid, or extract thereof), or a cell therefrom (e.g., primary cell or cell line, or extract thereof).

The term “prognosis” as used herein refers to a prediction of the probable course and outcome of a clinical condition or disease. A prognosis of a patient is usually made by evaluating factors or symptoms of a disease that are indicative of a favorable or unfavorable course or outcome of the disease.

### III. Coronary Artery Disease

Coronary artery disease (CAD), also called coronary heart disease (CHD), ischemic heart disease (IHD), myocardial ischemia, or simply heart disease, involves the reduction of blood flow to the heart muscle due to build-up of atherosclerotic plaque in the arteries of the heart. It is among the most common of the cardiovascular diseases. Types include stable angina, unstable angina, myocardial infarction, and sudden cardiac death. A common symptom of CAD is chest pain or discomfort, which may travel into the shoulder, arm, back, neck, or jaw. Occasionally it may feel like heartburn. Symptoms may occur with exercise or emotional stress, last less than a few minutes, and improve with rest. Shortness of breath may also occur and sometimes no symptoms are present. In many cases, the first sign is a heart attack. Other complications include heart failure or an abnormal heartbeat.

Risk factors for CAD include high blood pressure, smoking, diabetes, lack of exercise, obesity, high blood cholesterol, poor diet, depression, and excessive alcohol consumption. A number of tests may help with diagnoses, including, electrocardiogram, cardiac stress testing, coronary computed tomographic angiography, and coronary angiogram, among others.

Ways to reduce CAD risk include but are not limited to eating a healthy diet, regularly exercising, maintaining a healthy weight, and refraining from smoking. Medications for diabetes, high cholesterol, or high blood pressure are sometimes used to treat CAD. There is limited evidence for screening people who are at low risk and do not have symptoms. Treatment of CAD can involve the same measures as prevention of CAD. Some treatments for CAD may include administration of antiplatelets (including aspirin), beta blockers, or nitroglycerin. Procedures, such as percutaneous coronary intervention (PCI) or coronary artery bypass surgery (CABG) may be used to treat severe or advanced CAD. In subjects with stable CAD, it is unclear whether PCI or CABG in addition to the other treatments improves life expectancy or decreases heart attack risk.

CAD has a number of well determined risk factors. Some of these include high blood pressure, smoking, diabetes, lack of exercise, obesity, high blood cholesterol, poor diet, depression, family history, psychological stress and excessive alcohol. Smoking and obesity are associated with about 36% and 20% of cases, respectively. Smoking just one cigarette per day about doubles the risk of CAD. Lack of exercise has been linked to 7-12% of cases. Exposure to the herbicide Agent Orange may increase risk. Rheumatologic diseases such as rheumatoid arthritis, systemic lupus erythematosus, psoriasis, and psoriatic arthritis are independent risk factors as well.

About half of CAD cases are genetically linked. GWAS for CAD have discovered 306 independent signals. CAD heritability is significantly enriched in multiple cell types, including endothelial cells and vascular smooth muscle cells in the vessel wall, and hepatocytes, which influence cholesterol metabolism. At a few individual loci, noncoding risk variants have been shown to regulate the expression of key endothelial cell genes such as endothelial nitric oxide synthase (NOS3), endothelin 1 (EDN1), and others. However, prior to the present disclosure, it has been unclear which other genes in CAD GWAS loci might work together in which endothelial cell pathways to modulate disease risk.

### IV. CCM Pathway

The cerebral cavernous malformation (CCM) complex—comprised of KRIT1 (CCM1), CCM2, and PDCD10 (CCM3)—is named as such because loss-of-function mutations in any of these three genes cause cerebral vascular malformations, in which inactivation of the CCM complex in cerebral venous endothelial cells activates MEKK3/MEK5/ERK5 signaling and increases downstream activity of KLF2/4. Studies of the CCM complex in both venous and arterial endothelial cells have suggested it acts as a key signaling hub that senses and integrates information about cell-cell contacts, extracellular matrix interactions, and laminar blood flow, and regulates endothelial cell phenotypes in vitro such as cell migration and barrier integrity. See Whitehead, K. J. et al. The cerebral cavernous malformation signaling pathway promotes vascular integrity via Rho GTPases. Nat. Med. 15, 177-184 (2009), which is incorporated herein by reference in its entirety. Prior to the present disclosure, a possible link between the CCM complex and CAD has not been explored. Examples of other genes associated with the CCM pathway (i.e., CCM pathway associated genes include, without limitation, TLNRD1, HEG1, ITGB1BP1, ARPC2, CDC42, CDH5, DNM2, MEAF6, PDCD7, RHOA, KLF2, KLF4, MAP2K5, MAP3K3, MEF2A, and NFAT5. CCM pathway associated genes encompass the genes encoding proteins comprised in the CCM complex, including of KRIT1 (CCM1), CCM2, and PDCD10 (CCM3).

### V. Methods of Detecting Variant CCM Pathway Associated Genes

### Sample Collection and Preparation

The methods and compositions of the disclosure can be used to detect mutations in a CCM pathway gene and other mutations described herein using a biological sample obtained from an individual (e.g., a human individual, patient, or subject). A sample can be obtained from a subject suspected of having a mutated nucleic acid sequence, for example, from a tissue or a fluid sample from the subject. The methods provided herein can be performed using any sample containing nucleic acid. In some embodiments, the nucleic acid is deoxyribonucleic acid (DNA). In some embodiments, the nucleic acid is ribonucleic acid (RNA). The sample can be processed to release or otherwise make available a nucleic acid for detection as described herein. The nucleic acid (e.g., DNA or RNA) can be isolated from the sample according to any methods well-known to those of skill in the art. Such processing can include steps of nucleic acid manipulation, e.g., preparing a cDNA by reverse transcription of RNA from the biological sample. Thus, the nucleic acid to be assayed by the methods of the disclosure can be genomic DNA, cDNA, single stranded DNA or mRNA.

Examples of biological samples include tissue samples or any cell-containing or acellular bodily fluids. Biological samples can be obtained by standard procedures and can be used immediately or stored, under conditions appropriate for the type of biological sample, for later use.

Methods of obtaining test samples are well-known to those of skill in the art and include, but are not limited to, aspirations, tissue sections, drawing of blood or other fluids, surgical or needle biopsies, and the like. The test sample can be obtained from an individual or patient diagnosed as having a cardiovascular disorder or suspected being afflicted with a cardiovascular disorder. In some embodiments, the test sample is obtained from an individual or patient that has received one or more treatments for a cardiovascular disorder. The test sample can be a cell-containing liquid or a tissue. Samples can include, but are not limited to, amniotic fluid, biopsies, blood, blood cells, bone marrow, fine needle biopsy samples, peritoneal fluid, amniotic fluid, plasma, pleural fluid, saliva, semen, serum, tissue or tissue homogenates, frozen or paraffin sections of tissue. Samples can also be processed, such as sectioning of tissues, fractionation, purification, or cellular organelle separation.

If necessary, the sample can be collected or concentrated by centrifugation and the like. The cells of the sample can be subjected to lysis, such as by treatments with enzymes, heat, surfactants, ultrasonication, or a combination thereof. The lysis treatment is performed in order to obtain a sufficient amount of nucleic acid derived from the individual's cells to detect using a nucleic acid detection assay, e.g., a detection assay using PCR.

Methods of plasma and serum preparation are well-known in the art. Either “fresh” blood plasma or serum, or frozen (stored) and subsequently thawed plasma or serum can be used. Frozen (stored) plasma or serum should optimally be maintained at storage conditions of −20° C. to −70° C. until thawed and used. “Fresh” plasma or serum can be refrigerated or maintained on ice until used, with nucleic acid (e.g., RNA, DNA or total nucleic acid) extraction being performed as soon as possible.

### Nucleic Acid Extraction and Amplification

The nucleic acid to be assayed can be assayed directly from a biological sample or extracted from the biological sample prior to detection. As described herein, the biological sample can be any sample that contains a nucleic acid molecule, such as a fluid sample, a tissue sample, or a cell sample. The biological sample can be from a subject, which includes any animal, for example, a mammal. The subject can be a human, who can be a patient presenting to a medical provider for diagnosis or treatment of a disease. The volume of plasma or serum used in the extraction can be varied depending upon clinical intent, for example, 100 μL to one milliliter of plasma or serum may be used.

Various methods of extraction are suitable for isolating the DNA or RNA. In general, the aim is to separate DNA present in the nucleus of the cell from other cellular components. The isolation of nucleic acid usually involves lysis of tissue or cells. This process is essential for the destruction of protein structures and allows for release of nucleic acids from the nucleus. Lysis is typically carried out in a salt solution, containing detergents to denature proteins or proteases (enzymes digesting proteins), such as Proteinase K, or in some cases both. It results in the breakdown of cells and dissolving of membranes. Methods of DNA isolation include, but are not limited to, phenol: chloroform extraction, high salt precipitation, alkaline denaturation, ion exchange column chromatography, resin binding, and paramagnetic bead binding. See, e.g., Maniatis et al., Molecular Cloning, A Laboratory Manual, 2d, Cold Spring Harbor Laboratory Press, page 16.54 (1989). Numerous commercial kits that yield suitable DNA and RNA include, but are not limited to, QIAamp™ mini blood kit, Agencourt Genfind™, Roche Cobas®, Roche MagNA Pure®, or phenol: chloroform extraction using Eppendorf Phase Lock Gels®, and the NucliSens extraction kit (Biomerieux, Marcy l'Etoile, France).

Nucleic acid extracted from tissues, cells, plasma or serum can be amplified using nucleic acid amplification techniques well-known in the art. Many of these amplification methods can also be used to detect the presence of mutations simply by designing oligonucleotide primers or probes to interact with or hybridize to a particular target sequence in a specific manner (e.g., allele specific primers and/or probes or primers that flank target nucleic acids sequences). By way of example, but not by way of limitation, these techniques can include, but are not limited to, polymerase chain reaction (PCR), reverse transcriptase polymerase chain reaction (RT-PCR), real-time PCR (qPCR), nested PCR, ligase chain reaction (LCA) (see Abravaya, K., et al., Nucleic Acids Research, 23:675-682, (1995)), branched DNA signal amplification (Urdea, M. S., et al., AIDS, 7 (suppl 2): S11-S14, (1993)), amplifiable RNA reporters, Q-beta replication, transcription-based amplification system (TAS), boomerang DNA amplification, strand displacement activation (SDA), cycling probe technology, isothermal nucleic acid sequence based amplification (NASBA) (see Kievits, T. et al., J Virological Methods, 35:273-286, (1991)), Invader Technology, helicase dependent amplification (HDA) Amplification Refractory Mutation System (ARMS), and other sequence replication assays or signal amplification assays. Methods of amplification are well-known in the art.

A variety of amplification enzymes are well-known in the art and include, for example, DNA polymerase, RNA polymerase, reverse transcriptase, Q-beta replicase, thermostable DNA and RNA polymerases. Because these and other amplification reactions are catalyzed by enzymes, in a single step assay the nucleic acid releasing reagents and the detection reagents should not be potential inhibitors of amplification enzymes if the ultimate detection is to be amplification based.

PCR is a technique for exponentially making numerous copies of a specific template DNA sequence. The reaction consists of multiple amplification cycles (i.e., thermocycling) and is initiated using a pair of primer sequences that hybridize to the 5′ and 3′ ends of the sequence to be copied. The amplification cycle typically includes an initial denaturation (i.e. strand separation) of the target nucleic acid, typically at about 95° C., followed by up to 50 cycles or more of (1) denaturation, (2) annealing the primers to the target nucleic acid at a temperature determined by the melting point (Tm) of the region of homology between the primer and the target, and (3) extension at a temperature dependent on the polymerase, most commonly 72° C. An extended period of extension is typically performed at the end of the cycling. In each cycle of the reaction, the DNA sequence between the primers is copied. Primers can bind to the copied DNA as well as the original template sequence, so the total number of copies increases exponentially with time. PCR can be performed as according to Whelan, et al., J of Clin Micro, 33(3):556-561 (1995). An exemplary PCR reaction mixture includes two specific primers, dNTPs, approximately 0.25 U of thermostable polymerase, such as a Taq polymerase, and 1×PCR Buffer, typically containing a buffer (e.g., Tris), a salt (e.g. KCl) and magnesium (MgCl2). The Tm of a primer varies according to the length, G+C content, and the buffer conditions, among other factors. As used herein, Tm refers to that in the buffer used for the reaction of interest.

### Detection of Variant Sequences

Variant nucleic acids can be amplified prior to detection or can be detected directly during an amplification step (i.e., “real-time” methods). In some embodiments, the target sequence is amplified, and the resulting amplicon is detected by electrophoresis. In some embodiments, the specific mutation or variant is detected by sequencing the amplified nucleic acid, for example, Sanger sequencing or Next Generation Sequencing (NGS). Next-generation sequencing lowers the costs and greatly increases the speed over the industry standard detection methods. Examples of NGS include, but are not limited to, Massively Parallel Signature Sequencing (MPSS), Polony sequencing combined an in vitro paired-tag library with emulsion PCR, 454 pyrosequencing, Solexa sequencing, SOLID technology, DNA nanoball, Heliscope single molecule, Single molecule real time (SMRT) and ion semiconductor sequencing.

In some embodiments, the target sequence is amplified using a labeled primer such that the resulting amplicon is detectably labeled. In some embodiments, the primer is fluorescently labeled. In some embodiments, at least one allele-specific primer is used (e.g., a primer the spans the deletion breakpoint site, i.e., spans the junction formed by the 5′ and 3′ ends of the deletion).

In some embodiments, PCR amplification is performed in order to amplify a CCM2 gene, or variant, fragment, or exon thereof. In some embodiments, an exon comprising a putative variant (e.g., a nonsense variant described herein, e.g., rs2107732) is amplified.

In some embodiments, PCR amplification is performed in order to amplify a TLNRD1 gene, or variant, fragment, or exon thereof. In some embodiments, an exon comprising a putative variant is amplified.

In some embodiments, PCR amplification is performed in order to amplify a KRIT1 (CCM1) gene, or variant, fragment, or exon thereof. In some embodiments, an exon comprising a putative variant is amplified.

In some embodiments, PCR amplification is performed in order to amplify a PDCD10 (CCM3) gene, or variant, fragment, or exon thereof. In some embodiments, an exon comprising a putative variant is amplified.

In some embodiments, PCR amplification is performed in order to amplify a HEG1 gene, or variant, fragment, or exon thereof. In some embodiments, an exon comprising a putative variant is amplified.

In some embodiments, PCR amplification is performed in order to amplify a ITGB1BP1 gene, or variant, fragment, or exon thereof. In some embodiments, an exon comprising a putative variant is amplified.

In some embodiments, PCR amplification is performed in order to amplify a ARPC2 gene, or variant, fragment, or exon thereof. In some embodiments, an exon comprising a putative variant is amplified.

In some embodiments, PCR amplification is performed in order to amplify a CDC42 gene, or variant, fragment, or exon thereof. In some embodiments, an exon comprising a putative variant is amplified.

In some embodiments, PCR amplification is performed in order to amplify a CDH5 gene, or variant, fragment, or exon thereof. In some embodiments, an exon comprising a putative variant is amplified.

In some embodiments, PCR amplification is performed in order to amplify a DNM2 gene, or variant, fragment, or exon thereof. In some embodiments, an exon comprising a putative variant is amplified.

In some embodiments, PCR amplification is performed in order to amplify MEAF6 gene, or variant, fragment, or exon thereof. In some embodiments, an exon comprising a putative variant is amplified.

In some embodiments, PCR amplification is performed in order to amplify PDCD7 gene, or variant, fragment, or exon thereof. In some embodiments, an exon comprising a putative variant is amplified.

In some embodiments, PCR amplification is performed in order to amplify a RHOA gene, or variant, fragment, or exon thereof. In some embodiments, an exon comprising a putative variant is amplified.

In some embodiments, PCR amplification is performed in order to amplify a KLF2 gene, or variant, fragment, or exon thereof. In some embodiments, an exon comprising a putative variant is amplified.

In some embodiments, PCR amplification is performed in order to amplify a KLF4 gene, or variant, fragment, or exon thereof. In some embodiments, an exon comprising a putative variant is amplified.

In some embodiments, PCR amplification is performed in order to amplify a MAP2K5 gene, or variant, fragment, or exon thereof. In some embodiments, an exon comprising a putative variant is amplified.

In some embodiments, PCR amplification is performed in order to amplify a MAP3K3 gene, or variant, fragment, or exon thereof. In some embodiments, an exon comprising a putative variant is amplified.

In some embodiments, PCR amplification is performed in order to amplify a MEF2A gene, or variant, fragment, or exon thereof. In some embodiments, an exon comprising a putative variant is amplified.

In some embodiments, PCR amplification is performed in order to amplify a NFAT5 gene, or variant, fragment, or exon thereof. In some embodiments, an exon comprising a putative variant is amplified.

For the methods provided herein, a single primer can be used for detection, for example as in single nucleotide primer extension or allele-specific detection of nucleic acid containing the mutation, or a second primer can be used which can be upstream or downstream of the allele-specific primer. One or more of the primers used can be allele-specific primers. In some embodiments, the allele-specific primer contains a portion of wild-type sequence, for example, at least about 3-40 consecutive nucleotides of wild-type sequence.

In one embodiment, detection of a variant nucleic acid is performed using an RT-PCR assay, such as the TaqMan® assay, which is also known as the 5′ nuclease assay (U.S. Pat. Nos. 5,210,015 and 5,538,848) or Molecular Beacon probe (U.S. Pat. Nos. 5,118,801 and 5,312,728), or other stemless or linear beacon probe (Livak et al., 1995, PCR Method Appl., 4:357-362; Tyagi et al, 1996, Nature Biotechnology, 14:303-308; Nazarenko et al., 1997, Nucl. Acids Res., 25:2516-2521; U.S. Pat. Nos. 5,866,336 and 6,117,635). The TaqMan® assay detects the accumulation of a specific amplified product during PCR. The TaqMan® assay utilizes an oligonucleotide probe labeled with a fluorescent reporter dye and a quencher dye. The reporter dye is excited by irradiation at an appropriate wavelength, it transfers energy to the quencher dye in the same probe via a process called fluorescence resonance energy transfer (FRET). When attached to the probe, the excited reporter dye does not emit a signal. The proximity of the quencher dye to the reporter dye in the intact probe maintains a reduced fluorescence for the reporter. The reporter dye and quencher dye can be at the 5′ most and the 3′ most ends, respectively or vice versa. Alternatively, the reporter dye can be at the 5′ or 3′ most end while the quencher dye is attached to an internal nucleotide, or vice versa. In yet another embodiment, both the reporter dye and the quencher dye can be attached to internal nucleotides at a distance from each other such that fluorescence of the reporter dye is reduced.

During PCR, the 5′ nuclease activity of DNA polymerase cleaves the probe, thereby separating the reporter dye and the quencher dye and resulting in increased fluorescence of the reporter dye. Accumulation of PCR product is detected directly by monitoring the increase in fluorescence of the reporter dye. The DNA polymerase cleaves the probe between the reporter dye and the quencher dye only if the probe hybridizes to the target mutation-containing template which is amplified during PCR, and the probe is designed to hybridize to the target mutation site only if a particular mutation allele (e.g., SNP, insertion or deletion) is present.

TaqMan® primer and probe sequences can readily be determined using the variant and associated nucleic acid sequence information provided herein. A number of computer programs, such as Primer Express (Applied Biosystems, Foster City, Calif.), can be used to rapidly obtain optimal primer/probe sets. It will be apparent to one of skill in the art that such primers and probes for detecting the variants of the disclosure are useful in diagnostic or prognostic assays for cardiovascular disorders and related pathologies, and can be readily incorporated into a kit format. The disclosure also includes modifications of the TaqMan® assay well-known in the art such as the use of Molecular Beacon probes (U.S. Pat. Nos. 5,118,801 and 5,312,728) and other variant formats (U.S. Pat. Nos. 5,866,336 and 6,117,635).

Amplified fragments can be detected using standard gel electrophoresis methods. For example, in some embodiments, amplified fragments are separated on an agarose gel and stained with ethidium bromide by methods known in the art to detect amplified fragments.

In some embodiments, amplified nucleic acids are detected by hybridization with a mutation-specific probe. Probe oligonucleotides, complementary to a portion of the amplified target sequence, can be used to detect amplified fragments. Amplified nucleic acids for each of the target sequences can be detected simultaneously (i.e., in the same reaction vessel) or individually (i.e., in separate reaction vessels). In some embodiments, the amplified DNA is detected simultaneously, using two distinguishably-labeled, gene-specific oligonucleotide probes, one which hybridizes to the first target sequence and one which hybridizes to the second target sequence. Oligonucleotide probes can be designed which are between about 10 and about 100 nucleotides in length and hybridize to the amplified region. For example, oligonucleotides probes may be 12 to 70 nucleotides; such as 15-60 nucleotides in length; such as 15-25 nucleotides in length. The probe can be labeled.

In some embodiments, two or more assays are performed for detection of any of the variants described herein. In some embodiments, the identity of any of the variants described herein is confirmed by nucleic acid sequencing. Strategies for detecting or measuring a variant nucleic acid are well known in the art.

### VI. Methods of Treating Vascular Disease

### Methods of Treatment

The present disclosure provides methods of treating or preventing vascular disease (e.g., CAD) in a subject, the method comprising, consisting of, or consisting essentially of administering a therapy to the subject, such as a gene therapy.

Provided herein is a method for preventing or treating clinical or subclinical vascular disease (e.g., CAD) in a subject, the method comprising administering to the subject a therapeutically effective amount of a pharmacological agent capable of modulating the expression of one or more target gene in vascular endothelial cells, wherein the target gene is a Cerebral Cavernous Malformation (CCM) pathway gene, and/or a gene that regulates the function of the CCM pathway.

Non-limiting examples of CCM pathway genes include CCM2, KRIT1 (CCM1), and PDCD10 (CCM3). Non-limiting examples of genes that regulate the function of the CCM pathway include TLNRD1, HEG1, ITGB1BP1, ARPC2, CDC42, CDH5, DNM2, MEAF6, PDCD7, RHOA, KLF2, KLF4, MAP2K5, MAP3K3, MEF2A, and NFAT5. Target genes include TLNRD1, CCM2, HEG1, ITGB1BP1, KRIT1, PDCD10, ARPC2, CDC42, CDH5, DNM2, MEAF6, PDCD7, RHOA, KLF2, KLF4, MAP2K5, MAP3K3, MEF2A, and NFAT5.

### Gene Therapy

Genetic variants in over 300 genes have been linked to CAD. A newly emerging CAD locus has been identified herein at CCM pathway genes, and genes that regulate CCM pathway function. Non-limiting examples of CCM pathway genes include CCM2, KRIT1 (CCM1), and PDCD10 (CCM3). Non-limiting examples of genes that regulate the function of the CCM pathway include TLNRD1, HEG1, ITGB1BP1, ARPC2, CDC42, CDH5, DNM2, MEAF6, PDCD7, RHOA, KLF2, KLF4, MAP2K5, MAP3K3, MEF2A, and NFAT5. As described in the Examples section below, it was found, for the first time, that CCM pathway genes and CCM pathway regulators may provide a precise target for therapeutic intervention in CAD. In particular, it is hypothesized and shown that disruption of the CCM pathway, for example, by downregulation and/or upregulation of the function or expression of one or more CCM pathway genes and/or one or more genes that regulate the CCM pathway, is an effective treatment strategy for CAD.

Accordingly, in certain embodiments, a method of diagnosing and treating a subject having or at risk for vascular disease (e.g., CAD), comprises administering to the subject a therapeutically effective amount of a pharmacological agent, wherein the agent modulates expression or amount of a target CCM pathway gene product, proteins or peptides thereof in a target cell or tissue, as compared to a normal control.

In further embodiments, a method of treating a subject having or at risk for vascular disease, wherein the patient has at least one CCM2 nucleotide variant (NV) as compared to a control CCM2 nucleic acid sequence (such as that set forth in SEQ ID NO: 1), comprises administering to the patient a therapeutically effective amount of an agent wherein the agent modulates expression or amount of CCM pathway gene products, proteins or peptides thereof in a target cell or tissue.

In certain embodiments, provided is an agent for treating a patient or subject having or at risk for vascular disease (e.g., CAD), or a patient having clinical or subclinical CAD, wherein detecting certain genetic variants is predictive of whether a decrease in CCM pathway function is therapeutic for the patient or subject. In some embodiments, the agent is a pharmacological agent capable of modulating the expression of a target gene in vascular endothelial cells, wherein the target gene is a Cerebral Cavernous Malformation (CCM) pathway gene, or a gene that regulates the function of the CCM pathway. In some embodiments, the agent is a pharmacological agent capable of reducing the expression of a target gene in vascular endothelial cells, wherein the target gene is a Cerebral Cavernous Malformation (CCM) pathway gene, or a gene that regulates the function of the CCM pathway. In some embodiments, the agent is a pharmacological agent capable of increasing the expression of a target gene in vascular endothelial cells, wherein the target gene is a Cerebral Cavernous Malformation (CCM) pathway gene, or a gene that regulates the function of the CCM pathway.

In certain embodiments, a therapeutic agent for treatment or prevention of vascular disease modulates the expression or amounts of CCM2 in a cell, such as an endothelial cell, for example, an arterial endothelial cell. In some embodiments, compositions comprise nucleic acid sequences encoding CCM2, including, without limitation, one or more of (i) cDNA, (ii) CCM2-encoding RNA, mRNA, or chemically or structurally modified derivatives thereof (i.e., capped mRNAs, circular mRNAs, etc.), and (iii) sense and/or antisense sequences of CCM2.

In certain embodiments, the agent comprises one or more gene-editing or nuclease systems to delete or edit a CCM2 gene in subjects wherein a decrease in CCM2 expression or function would be therapeutic to the subject. In certain other embodiments, the gene-editing or nuclease system is used in conjunction with guide molecules to specifically target the CCM2 gene to reduce expression or activity of CCM2.

In certain embodiments, a therapeutic agent for treatment or prevention of vascular disease modulates the expression or amounts of TLNRD1 in a cell, such as an endothelial cell, for example, an arterial endothelial cell. In some embodiments, compositions comprise nucleic acid sequences encoding TLNRD1, including, without limitation, one or more of (i) cDNA, (ii) TLNRD1-encoding RNA, mRNA, or chemically or structurally modified derivatives thereof (i.e., capped mRNAs, circular mRNAs, etc.), and (iii) sense and/or antisense sequences of TLNRD1.

In certain embodiments, the agent comprises one or more gene-editing or nuclease systems to delete or edit a TLNRD1 gene in subjects wherein a decrease in TLNRD1 expression or function would be therapeutic to the subject. In certain other embodiments, the gene-editing or nuclease system is used in conjunction with guide molecules to specifically target the TLNRD1 gene to reduce expression or activity of TLNRD1.

In certain embodiments, a therapeutic agent for treatment or prevention of vascular disease modulates the expression or amounts of HEG1 in a cell, such as an endothelial cell, for example, an arterial endothelial cell. In some embodiments, compositions comprise nucleic acid sequences encoding HEG1, including, without limitation, one or more of (i) cDNA, (ii) HEG1-encoding RNA, mRNA, or chemically or structurally modified derivatives thereof (i.e., capped mRNAs, circular mRNAs, etc.), and (iii) sense and/or antisense sequences of HEG1.

In certain embodiments, the agent comprises one or more gene-editing or nuclease systems to delete or edit a HEG1 gene in subjects wherein a decrease in HEG1 expression or function would be therapeutic to the subject. In certain other embodiments, the gene-editing or nuclease system is used in conjunction with guide molecules to specifically target the HEG1 gene to reduce expression or activity of HEG1.

In certain embodiments, a therapeutic agent for treatment or prevention of vascular disease modulates the expression or amounts of ITGB1BP1 in a cell, such as an endothelial cell, for example, an arterial endothelial cell. In some embodiments, compositions comprise nucleic acid sequences encoding ITGB1BP1, including, without limitation, one or more of (i) cDNA, (ii) ITGB1BP1-encoding RNA, mRNA, or chemically or structurally modified derivatives thereof (i.e., capped mRNAs, circular mRNAs, etc.), and (iii) sense and/or antisense sequences of ITGB1BP1.

In certain embodiments, the agent comprises one or more gene-editing or nuclease systems to delete or edit a ITGB1BP1 gene in subjects wherein a decrease in ITGB1BP1 expression or function would be therapeutic to the subject. In certain other embodiments, the gene-editing or nuclease system is used in conjunction with guide molecules to specifically target the ITGB1BP1 gene to reduce expression or activity of ITGB1BP1.

In certain embodiments, a therapeutic agent for treatment or prevention of vascular disease modulates the expression or amounts of KRIT1 in a cell, such as an endothelial cell, for example, an arterial endothelial cell. In some embodiments, compositions comprise nucleic acid sequences encoding KRIT1, including, without limitation, one or more of (i) cDNA, (ii) KRIT1-encoding RNA, mRNA, or chemically or structurally modified derivatives thereof (i.e., capped mRNAs, circular mRNAs, etc.), and (iii) sense and/or antisense sequences of KRIT1.

In certain embodiments, the agent comprises one or more gene-editing or nuclease systems to delete or edit a KRIT1 gene in subjects wherein a decrease in KRIT1 expression or function would be therapeutic to the subject. In certain other embodiments, the gene-editing or nuclease system is used in conjunction with guide molecules to specifically target the KRIT1 gene to reduce expression or activity of KRIT1.

In certain embodiments, a therapeutic agent for treatment or prevention of vascular disease modulates the expression or amounts of PDCD10 in a cell, such as an endothelial cell, for example, an arterial endothelial cell. In some embodiments, compositions comprise nucleic acid sequences encoding PDCD10, including, without limitation, one or more of (i) cDNA, (ii) PDCD10-encoding RNA, mRNA, or chemically or structurally modified derivatives thereof (i.e., capped mRNAs, circular mRNAs, etc.), and (iii) sense and/or antisense sequences of PDCD10.

In certain embodiments, the agent comprises one or more gene-editing or nuclease systems to delete or edit a PDCD10 gene in subjects wherein a decrease in PDCD10 expression or function would be therapeutic to the subject. In certain other embodiments, the gene-editing or nuclease system is used in conjunction with guide molecules to specifically target the PDCD10 gene to reduce expression or activity of PDCD10.

In certain embodiments, a therapeutic agent for treatment or prevention of vascular disease modulates the expression or amounts of ARPC2 in a cell, such as an endothelial cell, for example, an arterial endothelial cell. In some embodiments, compositions comprise nucleic acid sequences encoding ARPC2, including, without limitation, one or more of (i) cDNA, (ii) ARPC2-encoding RNA, mRNA, or chemically or structurally modified derivatives thereof (i.e., capped mRNAs, circular mRNAs, etc.), and (iii) sense and/or antisense sequences of ARPC2.

In certain embodiments, the agent comprises one or more gene-editing or nuclease systems to delete or edit a ARPC2 gene in subjects wherein a decrease in ARPC2 expression or function would be therapeutic to the subject. In certain other embodiments, the gene-editing or nuclease system is used in conjunction with guide molecules to specifically target the ARPC2 gene to reduce expression or activity of ARPC2.

In certain embodiments, a therapeutic agent for treatment or prevention of vascular disease modulates the expression or amounts of CDC42 in a cell, such as an endothelial cell, for example, an arterial endothelial cell. In some embodiments, compositions comprise nucleic acid sequences encoding CDC42, including, without limitation, one or more of (i) cDNA, (ii) CDC42-encoding RNA, mRNA, or chemically or structurally modified derivatives thereof (i.e., capped mRNAs, circular mRNAs, etc.), and (iii) sense and/or antisense sequences of CDC42.

In certain embodiments, the agent comprises one or more gene-editing or nuclease systems to delete or edit a CDC42 gene in subjects wherein a decrease in CDC42 expression or function would be therapeutic to the subject. In certain other embodiments, the gene-editing or nuclease system is used in conjunction with guide molecules to specifically target the CDC42 gene to reduce expression or activity of CDC42.

In certain embodiments, a therapeutic agent for treatment or prevention of vascular disease modulates the expression or amounts of CDH5 in a cell, such as an endothelial cell, for example, an arterial endothelial cell. In some embodiments, compositions comprise nucleic acid sequences encoding CDH5, including, without limitation, one or more of (i) cDNA, (ii) CDH5-encoding RNA, mRNA, or chemically or structurally modified derivatives thereof (i.e., capped mRNAs, circular mRNAs, etc.), and (iii) sense and/or antisense sequences of CDH5.

In certain embodiments, the agent comprises one or more gene-editing or nuclease systems to delete or edit a CDH5 gene in subjects wherein a decrease in CDH5 expression or function would be therapeutic to the subject. In certain other embodiments, the gene-editing or nuclease system is used in conjunction with guide molecules to specifically target the CDH5 gene to reduce expression or activity of CDH5.

In certain embodiments, a therapeutic agent for treatment or prevention of vascular disease modulates the expression or amounts of DNM2 in a cell, such as an endothelial cell, for example, an arterial endothelial cell. In some embodiments, compositions comprise nucleic acid sequences encoding DNM2, including, without limitation, one or more of (i) cDNA, (ii) DNM2-encoding RNA, mRNA, or chemically or structurally modified derivatives thereof (i.e., capped mRNAs, circular mRNAs, etc.), and (iii) sense and/or antisense sequences of DNM2.

In certain embodiments, the agent comprises one or more gene-editing or nuclease systems to delete or edit a DNM2 gene in subjects wherein a decrease in DNM2 expression or function would be therapeutic to the subject. In certain other embodiments, the gene-editing or nuclease system is used in conjunction with guide molecules to specifically target the DNM2 gene to reduce expression or activity of DNM2.

In certain embodiments, a therapeutic agent for treatment or prevention of vascular disease modulates the expression or amounts of MEAF6 in a cell, such as an endothelial cell, for example, an arterial endothelial cell. In some embodiments, compositions comprise nucleic acid sequences encoding MEAF6, including, without limitation, one or more of (i) cDNA, (ii) MEAF6-encoding RNA, mRNA, or chemically or structurally modified derivatives thereof (i.e., capped mRNAs, circular mRNAs, etc.), and (iii) sense and/or antisense sequences of MEAF6.

In certain embodiments, the agent comprises one or more gene-editing or nuclease systems to delete or edit a MEAF6 gene in subjects wherein a decrease in MEAF6 expression or function would be therapeutic to the subject. In certain other embodiments, the gene-editing or nuclease system is used in conjunction with guide molecules to specifically target the MEAF6 gene to reduce expression or activity of MEAF6.

In certain embodiments, a therapeutic agent for treatment or prevention of vascular disease modulates the expression or amounts of PDCD7 in a cell, such as an endothelial cell, for example, an arterial endothelial cell. In some embodiments, compositions comprise nucleic acid sequences encoding PDCD7, including, without limitation, one or more of (i) cDNA, (ii) PDCD7-encoding RNA, mRNA, or chemically or structurally modified derivatives thereof (i.e., capped mRNAs, circular mRNAs, etc.), and (iii) sense and/or antisense sequences of PDCD7.

In certain embodiments, the agent comprises one or more gene-editing or nuclease systems to delete or edit a PDCD7 gene in subjects wherein a decrease in PDCD7 expression or function would be therapeutic to the subject. In certain other embodiments, the gene-editing or nuclease system is used in conjunction with guide molecules to specifically target the PDCD7 gene to reduce expression or activity of PDCD7.

In certain embodiments, a therapeutic agent for treatment or prevention of vascular disease modulates the expression or amounts of RHOA in a cell, such as an endothelial cell, for example, an arterial endothelial cell. In some embodiments, compositions comprise nucleic acid sequences encoding RHOA, including, without limitation, one or more of (i) cDNA, (ii) RHOA-encoding RNA, mRNA, or chemically or structurally modified derivatives thereof (i.e., capped mRNAs, circular mRNAs, etc.), and (iii) sense and/or antisense sequences of RHOA.

In certain embodiments, the agent comprises one or more gene-editing or nuclease systems to delete or edit a RHOA gene in subjects wherein a decrease in RHOA expression or function would be therapeutic to the subject. In certain other embodiments, the gene-editing or nuclease system is used in conjunction with guide molecules to specifically target the RHOA gene to reduce expression or activity of RHOA.

In certain embodiments, a therapeutic agent for treatment or prevention of vascular disease modulates the expression or amounts of KLF2 in a cell, such as an endothelial cell, for example, an arterial endothelial cell. In some embodiments, compositions comprise nucleic acid sequences encoding KLF2, including, without limitation, one or more of (i) cDNA, (ii) KLF2-encoding RNA, mRNA, or chemically or structurally modified derivatives thereof (i.e., capped mRNAs, circular mRNAs, etc.), and (iii) sense and/or antisense sequences of KLF2.

In certain embodiments, the agent comprises one or more gene-editing or nuclease systems to delete or edit a KLF2 gene in subjects wherein a decrease in KLF2 expression or function would be therapeutic to the subject. In certain other embodiments, the gene-editing or nuclease system is used in conjunction with guide molecules to specifically target the KLF2 gene to reduce expression or activity of KLF2.

In certain embodiments, a therapeutic agent for treatment or prevention of vascular disease modulates the expression or amounts of KLF4 in a cell, such as an endothelial cell, for example, an arterial endothelial cell. In some embodiments, compositions comprise nucleic acid sequences encoding KLF4, including, without limitation, one or more of (i) cDNA, (ii) KLF4-encoding RNA, mRNA, or chemically or structurally modified derivatives thereof (i.e., capped mRNAs, circular mRNAs, etc.), and (iii) sense and/or antisense sequences of KLF4.

In certain embodiments, the agent comprises one or more gene-editing or nuclease systems to delete or edit a KLF4 gene in subjects wherein a decrease in KLF4 expression or function would be therapeutic to the subject. In certain other embodiments, the gene-editing or nuclease system is used in conjunction with guide molecules to specifically target the KLF4 gene to reduce expression or activity of KLF4.

In certain embodiments, a therapeutic agent for treatment or prevention of vascular disease modulates the expression or amounts of MAP2K5 in a cell, such as an endothelial cell, for example, an arterial endothelial cell. In some embodiments, compositions comprise nucleic acid sequences encoding MAP2K5, including, without limitation, one or more of (i) cDNA, (ii) MAP2K5-encoding RNA, mRNA, or chemically or structurally modified derivatives thereof (i.e., capped mRNAs, circular mRNAs, etc.), and (iii) sense and/or antisense sequences of MAP2K5.

In certain embodiments, the agent comprises one or more gene-editing or nuclease systems to delete or edit a MAP2K5 gene in subjects wherein a decrease in MAP2K5 expression or function would be therapeutic to the subject. In certain other embodiments, the gene-editing or nuclease system is used in conjunction with guide molecules to specifically target the MAP2K5 gene to reduce expression or activity of MAP2K5.

In certain embodiments, a therapeutic agent for treatment or prevention of vascular disease modulates the expression or amounts of MAP3K3 in a cell, such as an endothelial cell, for example, an arterial endothelial cell. In some embodiments, compositions comprise nucleic acid sequences encoding MAP3K3, including, without limitation, one or more of (i) cDNA, (ii) MAP3K3-encoding RNA, mRNA, or chemically or structurally modified derivatives thereof (i.e., capped mRNAs, circular mRNAs, etc.), and (iii) sense and/or antisense sequences of MAP3K3.

In certain embodiments, the agent comprises one or more gene-editing or nuclease systems to delete or edit a MAP3K3 gene in subjects wherein a decrease in MAP3K3 expression or function would be therapeutic to the subject. In certain other embodiments, the gene-editing or nuclease system is used in conjunction with guide molecules to specifically target the MAP3K3 gene to reduce expression or activity of MAP3K3.

In certain embodiments, a therapeutic agent for treatment or prevention of vascular disease modulates the expression or amounts of MEF2A in a cell, such as an endothelial cell, for example, an arterial endothelial cell. In some embodiments, compositions comprise nucleic acid sequences encoding MEF2A, including, without limitation, one or more of (i) cDNA, (ii) MEF2A-encoding RNA, mRNA, or chemically or structurally modified derivatives thereof (i.e., capped mRNAs, circular mRNAs, etc.), and (iii) sense and/or antisense sequences of MEF2A.

In certain embodiments, the agent comprises one or more gene-editing or nuclease systems to delete or edit a MEF2A gene in subjects wherein a decrease in MEF2A expression or function would be therapeutic to the subject. In certain other embodiments, the gene-editing or nuclease system is used in conjunction with guide molecules to specifically target the MEF2A gene to reduce expression or activity of MEF2A.

In certain embodiments, a therapeutic agent for treatment or prevention of vascular disease modulates the expression or amounts of NFAT5 in a cell, such as an endothelial cell, for example, an arterial endothelial cell. In some embodiments, compositions comprise nucleic acid sequences encoding NFAT5, including, without limitation, one or more of (i) cDNA, (ii) NFAT5-encoding RNA, mRNA, or chemically or structurally modified derivatives thereof (i.e., capped mRNAs, circular mRNAs, etc.), and (iii) sense and/or antisense sequences of NFAT5.

In certain embodiments, the agent comprises one or more gene-editing or nuclease systems to delete or edit a NFAT5 gene in subjects wherein a decrease in NFAT5 expression or function would be therapeutic to the subject. In certain other embodiments, the gene-editing or nuclease system is used in conjunction with guide molecules to specifically target the NFAT5 gene to reduce expression or activity of NFAT5.

A gene editing system can include any suitable nuclease system, including, for example, clustered regularly interspaced short palindromic repeat (CRISPR) nucleases, Argonaute family of endonucleases, zinc-finger nucleases (ZFNs), transcription activator-like effector nucleases (TALENs), meganucleases, other endo- or exo-nucleases, or combinations thereof. See Schiffer, 2012, J Virol 88(17):8920-8936, incorporated by reference. In certain embodiments, the system is an Argonaute nuclease system. In certain embodiments, the system is a CRISPR system, such as a CRISPR-Cas system.

In certain embodiments, the gene editing system or agent comprises a Clustered Regularly Interspaced Short Palindromic Repeat (CRISPR)-associated endonuclease/Cas (CRISPR/Cas).

In certain embodiments, the CRISPR/Cas comprises catalytically deficient Cas protein (dCas), orthologs, homologs, mutant variants or fragments thereof. In some embodiments, the CRISPR-Cas system comprises a catalytically deficient Cas protein (dCas), ortholog, homolog, mutant, variant, or fragments thereof, linked (i.e., fused) to an effector domain, such as a repression domain, such as, without limitation, KRAB, DNMT1, or HDAC.

The compositions disclosed herein may include nucleic acids encoding a CRISPR-associated endonuclease, such as Cas9. In bacteria, the CRISPR/Cas loci encode RNA-guided adaptive immune systems against mobile genetic elements (viruses, transposable elements and conjugative plasmids). Three types (I-III) of CRISPR systems have been identified. CRISPR clusters contain spacers, the sequences complementary to antecedent mobile elements. CRISPR clusters are transcribed and processed into mature CRISPR RNA (crRNA). The CRISPR-associated endonuclease, Cas9, belongs to the type II CRISPR/Cas system and has strong endonuclease activity to cut target DNA. Cas9 is guided by a mature crRNA that contains about 20 base pairs (bp) of unique target sequence (called spacer) and a trans-activated small RNA (tracrRNA) that serves as a guide for ribonuclease III-aided processing of pre-crRNA. The crRNA: tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM). The crRNA and tracrRNA can be expressed separately or engineered into an artificial fusion small guide RNA or single guide RNA (sgRNA) via a synthetic stem loop (AGAAAU) to mimic the natural crRNA/tracrRNA duplex. Such sgRNA, like shRNA, can be synthesized or in vitro transcribed for direct RNA transfection or expressed from U6 or H1-promoted RNA expression vector, although cleavage efficiencies of the artificial sgRNA are lower than those for systems with the crRNA and tracrRNA expressed separately.

Exemplary sgRNAs that can be used to target a gene editing system specifically to a target locus in a cell are provided in Table 1.

TABLE 1
Exemplary sgRNA sequences

| SEQ ID NO: | Gene | sgRNA Sequence |
|---|---|---|
| 3 | CCM2 | GAGACTTCGGGCCCGGCTGGC |
| 4 | CCM2 | GAGCCGCTACATGCTCGACCC |
| 5 | CCM2 | GATCGCCGCGTGCGGCTCCCG |
| 6 | CCM2 | GATGAGCGACAACCGTGGGCG |
| 7 | CCM2 | GCAAGAAGGTGAGCGTGCGCG |
| 8 | CCM2 | GCATGTAGCGGCTGCTGGCGG |
| 9 | CCM2 | GCGCGGCGATATGGAAGAGGA |
| 10 | CCM2 | GCGGAGACTTCGGGCCCGGC |
| 11 | CCM2 | GCTCTTCCATATCGCCGCGTG |
| 12 | CCM2 | GCTGCTGGCGGCGGGGCTCC |
| 13 | CCM2 | GGATGAGCGACAACCGTGGG |
| 14 | CCM2 | GGCAAGAAGGTGAGCGTGCG |
| 15 | CCM2 | GGCCCCACCTTCCCCAGCGC |
| 16 | CCM2 | GTCGAGCATGTAGCGGCTGC |
| 17 | CCM2 | GTGAGGATGAGCGACAACCG |
| 18 | CCM2 | GAAGTCGGCCGCCGTAAAGA |
| 19 | CCM2 | GAGCCTGCCTCGCGTCCAGGG |
| 20 | CCM2 | GATTTGCCGCCATCTTTACGG |
| 21 | CCM2 | GCAGGTCTCTGAAAAGGGGAC |
| 22 | CCM2 | GCCCGCGTCCTTCAGGAGAGC |
| 23 | CCM2 | GCGGCCGTGCAGAAGCGAAGT |
| 24 | CCM2 | GCGTCCAGGGAGGCGGAGCTC |
| 25 | CCM2 | GCTCCTGAAGGACGCGGGGCG |
| 26 | CCM2 | GCTGAGTGGGCGGAGCGAAC |
| 27 | CCM2 | GCTGCCTCGCGTCCAGGGAGG |
| 28 | CCM2 | GGAGCTCAGGTCTCTGAAAA |
| 29 | CCM2 | GTCCCGCGCGCGCTGAGTGGG |
| 30 | CCM2 | GTCCGGCTCTCCTGAAGGACG |
| 31 | CCM2 | GTCTCTGAAAAGGGGACGGG |
| 32 | CCM2 | GTGCAGAAGCGAAGTAGGGGT |
| 33 | HEG1 | GAAGTTATCGGGAGCCGCGGG |
| 34 | HEG1 | GAGGCTCAGCGCGCGGCGAGC |
| 35 | HEG1 | GAGTTATCGGGAGCCGCGGGA |
| 36 | HEG1 | GCCCGCCCGCGCTCACATGCC |
| 37 | HEG1 | GCCTCACTCCGCTCACTTCCC |
| 38 | HEG1 | GCGCGCCGGGCATGTGAGCG |
| 39 | HEG1 | GCGCGCCGGGCATGTGAGCGC |
| 40 | HEG1 | GCGCGGCGAGGCCATGGTGA |
| 41 | HEG1 | GCGCTCACATGCCCGGCGCG |
| 42 | HEG1 | GCGGGACCCGCCGCCTTCCC |
| 43 | HEG1 | GCTCACATGCCCGGCGCGCG |
| 44 | HEG1 | GCTCAGCGCGCGGCGAGCCG |
| 45 | HEG1 | GGCTCAGCGCGCGGCGAGCC |
| 46 | HEG1 | GTCAAAAGTTATCGGGAGCCG |
| 47 | HEG1 | GTGGCCTCGCCGCGCGCCTCG |
| 48 | ITGB1BP1 | GAAGGCCGCGGCACTCCCACG |
| 49 | ITGB1BP1 | GAAGTCCGCAACCCGGGGAT |
| 50 | ITGB1BP1 | GACCCCGAAGTCCGCAACCC |
| 51 | ITGB1BP1 | GACTTGGGGTCTTGGAACCGG |
| 52 | ITGB1BP1 | GAGCACAACTTCCGTCACTC |
| 53 | ITGB1BP1 | GAGTAGCTGCCTGGGCGCCAA |
| 54 | ITGB1BP1 | GAGTCTAGTGCCGCGGGCTC |
| 55 | ITGB1BP1 | GCCACTTGGGGTCTTGGAACC |
| 56 | ITGB1BP1 | GCCCTCCTGCCCACGTCCGC |
| 57 | ITGB1BP1 | GCCTCCCCTCGACTTCCGGTC |
| 58 | ITGB1BP1 | GCGCAGCCGCGGGCCCATCCC |
| 59 | ITGB1BP1 | GTCCCGTTCCCGCTCCAGCGC |
| 60 | ITGB1BP1 | GTGCGCATAGTCTTGGAGGCG |
| 61 | ITGB1BP1 | GTGGAAAAGCCGGCGCTGGAG |
| 62 | ITGB1BP1 | GTGGTGGGAAGCCTCGCGAG |
| 63 | KRIT1 | GAAGGACAGTAAAGAGCAGGT |
| 64 | KRIT1 | GAGAGCTTCCGGGTCGGCGGC |
| 65 | KRIT1 | GAGCAGGAACGGCAGCAGCC |
| 66 | KRIT1 | GAGGGAAGGACAGTAAAGAGC |
| 67 | KRIT1 | GCACCCGCGCCCCAAAAGCTC |
| 68 | KRIT1 | GCACCCTCTCCCGAAACTACG |
| 69 | KRIT1 | GCCAACCGCGAGGCTGCAAA |
| 70 | KRIT1 | GCGATTTCATAGCCACGGTAA |
| 71 | KRIT1 | GCGGTGAAAGAGCTTCCGGGT |
| 72 | KRIT1 | GCTAAGGCCTTTGAAGACCT |
| 73 | KRIT1 | GTAGTTTCGGGAGAGGGTGA |
| 74 | KRIT1 | GTCACACCCTTTGCAGCCTCG |
| 75 | KRIT1 | GTCCCGAAACTACGGGGTCCC |
| 76 | KRIT1 | GTTCATAGCCACGGTAACGGC |
| 77 | KRIT1 | GTTTCCACCCCGCCGTTACCG |
| 78 | TLNRD1 | GAAAAGATTTCTATAGGCTCC |
| 79 | TLNRD1 | GAAACTGAAAGGAAGGGCGG |
| 80 | TLNRD1 | GAAAGCCTCCGGCAGCCCCTG |
| 81 | TLNRD1 | GAGAAACTGAAAGGAAGGGCG |
| 82 | TLNRD1 | GATTTCTATAGGCTCCAGGG |
| 83 | TLNRD1 | GCCGCGCGCGGAGAGGAAGC |
| 84 | TLNRD1 | GCCTCCGGCAGCCCCTGCGGG |
| 85 | TLNRD1 | GCGGGCAGAAACTGAAAGGA |
| 86 | TLNRD1 | GCTCCAGGGAGGTTACGGCCG |
| 87 | TLNRD1 | GCTCCGAGGTGAAGCCGCGCG |
| 88 | TLNRD1 | GGAAGCGGGATTTAAACGAA |
| 89 | TLNRD1 | GTCAGTTTCTGCCCGCTCGCT |
| 90 | TLNRD1 | GTCCCGTCCGCGCCTCGCCCC |
| 91 | TLNRD1 | GTGGCTGCGCCGCCGCCCGCA |
| 92 | TLNRD1 | GTTCACCTCGGAGCGCGGCCG |
| 93 | PDCD10 (CCM3) | GACCGAGCAGAAGAGGTCTA |
| 94 | PDCD10 (CCM3) | GCCGCTTTACGCCACTCGCGT |
| 95 | ARPC2 | GAAACAAGGGCCGGTGAGGG |
| 96 | ARPC2 | GAAGGGTAAGACGAACGCGC |
| 97 | ARPC2 | GAGCCGCCGCCATGATCCTGC |
| 98 | ARPC2 | GAGTTCGAGAACGCGGCCGCC |
| 99 | ARPC2 | GCACCTCCAGCAGGATCATGG |
| 100 | ARPC2 | GCCCCACCCAGCCCCACTCAG |
| 101 | ARPC2 | GCCCCAGGGGAAGGAGAAACA |
| 102 | ARPC2 | GCCTCCCTTACCCACCCTCAC |
| 103 | ARPC2 | GCGAGCGTCTCCTCGATGATG |
| 104 | ARPC2 | GCGCGCTCAAGTTCGAGAACG |
| 105 | ARPC2 | GCTCCAGCAGGATCATGGCGG |
| 106 | ARPC2 | GGAGAAACAAGGGCCGGTGA |
| 107 | ARPC2 | GGGGAAGGAGAAACAAGGGC |
| 108 | ARPC2 | GTACCCCACCCAGCCCCACTC |
| 109 | ARPC2 | GTGCGGTTGTTCACCTCCAGC |
| 110 | CDC42 | GAGTTGGGTGCCCGCGGAAGT |
| 111 | CDC42 | GCCCGCCTGTCGCGCACGCGC |
| 112 | CDC42 | GCCCGCGCGTGCGCGACAGG |
| 113 | CDC42 | GCCTGTTCCTCCCACTTCCGC |
| 114 | CDC42 | GCGCGCAGGAGACGCACAGT |
| 115 | CDC42 | GCGGGGCCCGAGGTTCCCCGC |
| 116 | CDC42 | GGAGAAGCTGAGGTGAGTGC |
| 117 | CDC42 | GGCGTTGGCAGCACTGCGCG |
| 118 | CDC42 | GGGCGGAGCCAGGGCAGTCC |
| 119 | CDC42 | GGGGCGTTGGCAGCACTGCG |
| 120 | CDC42 | GTAGGGTGGCAGCTCCCGCG |
| 121 | CDC42 | GTCACCTCAGCTTCTCCACCG |
| 122 | CDC42 | GTCCGTCCTCTCCCAGTCCTC |
| 123 | CDC42 | GTCTCCTCGGCTGCCGGACA |
| 124 | CDC42 | GTGCCCCTGTCCGGCAGCCG |
| 125 | CDH5 | GAGCCAGCCCAGCCCTCACAA |
| 126 | CDH5 | GAGCCAGGCCTCCCTCGCCAG |
| 127 | CDH5 | GCAATAACAGGAAACCATCCC |
| 128 | CDH5 | GCATCCCTCAGCCCACAGGCA |
| 129 | CDH5 | GCCAGCTGGAAAACCTGAAG |
| 130 | CDH5 | GCCCACAGGCACGGTGAGTGG |
| 131 | CDH5 | GCCCCGCTGGCGAGGGAGGCC |
| 132 | CDH5 | GCGTCCAGGGCTGAGCGTGAG |
| 133 | CDH5 | GCTCACCGTGCCTGTGGGCTG |
| 134 | CDH5 | GCTGCAGATAGGCAGGCCCTG |
| 135 | CDH5 | GGAGCCACACCCCGCTGGCG |
| 136 | CDH5 | GGCTCCCCTCCAAAGACGGT |
| 137 | CDH5 | GTAACAGGAAACCATCCCAGG |
| 138 | CDH5 | GTCAGCCGACCGTCTTTGGA |
| 139 | CDH5 | GTTGGACTGCCTGTCCGTCCA |
| 140 | DNM2 | GACCCGAGCGACCTCCCGCCC |
| 141 | DNM2 | GAGCGCAGGGCGCTCGGGCC |
| 142 | DNM2 | GCAACGGCTACAGACGCCGC |
| 143 | DNM2 | GCCCTCAACGACCTGGCCCCG |
| 144 | DNM2 | GCCGCCGACCCTCAACGACC |
| 145 | DNM2 | GCCGTTGCTCCCCGCCCGCC |
| 146 | DNM2 | GCGGGCGTCTTGCCGAGGCCC |
| 147 | DNM2 | GCGTCTTGCCGAGGCCCGGGC |
| 148 | DNM2 | GCTCACGGTCGCCGCCTCATC |
| 149 | DNM2 | GCTCGGCAAGACGCCCGCTCC |
| 150 | DNM2 | GGCGACCGTGAGGCCGAGCC |
| 151 | DNM2 | GTATCCGAGTGGCGGTGCAGC |
| 152 | DNM2 | GTATTGGCTAGTGTATCCGAG |
| 153 | DNM2 | GTGCAGCAGGCCAGTCGCGTG |
| 154 | DNM2 | GTGTCGCCTGAGAACCGGATG |
| 155 | MEAF6 | GACAGGGGTTGCACGGCATG |
| 156 | MEAF6 | GATCTCTAGAGCTCTCCGAGA |
| 157 | MEAF6 | GCCCCGGCCTTAGCGAAGCT |
| 158 | MEAF6 | GCCGCCCGCCTCAGCCCAACA |
| 159 | MEAF6 | GCGAGCTCCGCCAGCTCCCGC |
| 160 | MEAF6 | GCGCAGATCCCGGACACCCGG |
| 161 | MEAF6 | GCGCCGTGCCGAGCTTCGCTA |
| 162 | MEAF6 | GCGCGCGCAGGCGCAGAGCG |
| 163 | MEAF6 | GCGTGAAGCGGAAGCAGGAGC |
| 164 | MEAF6 | GGACACCCGGCGGGAGCTGG |
| 165 | MEAF6 | GGCCTTAGCGAAGCTCGGCA |
| 166 | MEAF6 | GGCGGCGCCGCCGCAGATCC |
| 167 | MEAF6 | GGCGGTCCCTGCGCCGCCAG |
| 168 | MEAF6 | GGGCTAGGACAGGGGTTGCA |
| 169 | MEAF6 | GTGCATCGCCATGTTGGGCTG |
| 170 | PDCD7 | GAAGCATCTCCGTCGTGAACA |
| 171 | PDCD7 | GAGAAGTGACAGGAATCTGAG |
| 172 | PDCD7 | GCCTGCCACCATTCTTCGGCC |
| 173 | PDCD7 | GCGACCCTGGCCGAAGAATGG |
| 174 | PDCD7 | GCGGTGGGCCTGGGCGACCC |
| 175 | PDCD7 | GCGTAACGACGTCACTTCCGT |
| 176 | PDCD7 | GCTGGCCGAAGAATGGTGGCA |
| 177 | PDCD7 | GGAAAAGGGCCGGGCCGCTG |
| 178 | PDCD7 | GGAAGGCTCAGTGCATAAGT |
| 179 | PDCD7 | GGACAGCCGAAAGGAGCAGG |
| 180 | PDCD7 | GGCGGTGGCGGACAGCCGAA |
| 181 | PDCD7 | GTCCGTCGGAAGGTTCTCATG |
| 182 | PDCD7 | GTGTAGACTCGTCCCTCCGGA |
| 183 | PDCD7 | GTTATGCACTGAGCCTTCCGG |
| 184 | PDCD7 | GTTCCGGAGGGACGAGTCTAC |
| 185 | RHOA | GACTAACGAGAGAACCGACGG |
| 186 | RHOA | GAGAACCGACGGAGGACCGC |
| 187 | RHOA | GCAGCGGGCTCGAGCCGGCAC |
| 188 | RHOA | GCGAGCCCGCTGGGCACTCGG |
| 189 | RHOA | GCGCTACCCTCCCGCCGCCCG |
| 190 | RHOA | GCTGTGCCGGCTCGAGCCCGC |
| 191 | RHOA | GGTAGCTGAAGACCAGACCG |
| 192 | RHOA | GTGAGTGCGCGCGCGTGCGC |
| 193 | RHOA | GAACCAATCATTGCAGCCGCG |
| 194 | RHOA | GACCAATCATTGCAGCCGCGT |
| 195 | RHOA | GAGTTCCCGTGATGCCCCACG |
| 196 | RHOA | GCAGCCGCGTGGGGCATCAC |
| 197 | RHOA | GCTCCGGGGCTATAAAAGACC |
| 198 | RHOA | GGGGCATCACGGGAACTCCG |
| 199 | RHOA | GTGCAGCCGCGTGGGGCATCA |
| 200 | KLF2 | GGACCCGGGGAGAAAGGACG |
| 201 | KLF2 | GCCGCGGTATATAAGCCGGC |
| 202 | MAP2K5 | GCCGAGGCCGCGCGGACTGG |
| 203 | MAP2K5 | GTCTGCCCCACCCGGAGACAC |
| 204 | MAP3K3 | GTTCCTGAGGTGGAGAACGG |
| 205 | MAP3K3 | GCCAATAACAAGAAGGAAGT |
| 206 | MEF2A | GCGGCGCGAAGCGCTGGTGG |
| 207 | MEF2A | GACTGAATTATCCTCTCGGT |
| 208 | NFAT5 | GGCCTCGCTTCCTGCCGGCG |
| 209 | NFAT5 | GGTCCCCGTCCCGCCGGGGG |

In some embodiments, sgRNAs of the present disclosure are capable of targeting a gene editing system to target locus, resulting in modulation of expression of a gene of the locus. In some embodiments, the modulation of expression is a reduction of expression.

In some embodiments, a CRISPR/Cas system can be a type I, a type II, or a type III system. Non-limiting examples of suitable CRISPR/Cas proteins include Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

The Cas9 can be orthologous. Six smaller Cas9 orthologs have been used and reports have shown that Cas9 from Staphylococcus aureus (SaCas9) can edit the genome with efficiencies similar to those of SpCas9, while being more than 1 kilobase shorter.

In addition to the wild type and variant Cas9 endonucleases described, embodiments of the disclosure also encompass CRISPR systems including “enhanced-specificity” S. pyogenes Cas9 variants (eSpCas9), which dramatically reduce off target cleavage. These variants are engineered with alanine substitutions to neutralize positively charged sites in a groove that interacts with the non-target strand of DNA. The aim of this modification is to reduce interaction of Cas9 with the non-target strand, thereby encouraging re-hybridization between target and non-target strands. The effect of this modification is a requirement for more stringent Watson-Crick pairing between the gRNA and the target DNA strand, which limits off-target cleavage (Slaymaker, I. M. et al. (2015) DOI: 10.1126/science.aad5227).

In certain embodiments, three variants found to have the best cleavage efficiency and fewest off-target effects: SpCas9 (K855A), SpCas9 (K810A/K1003A/R1060A) (a.k.a. eSpCas9 1.0), and SpCas9 (K848A/K1003A/R1060A) (a.k.a. eSPCas9 1.1) are employed in the compositions. The disclosure is by no means limited to these variants, and also encompasses all Cas9 variants (Slaymaker, I. M. et al. (2015)). The disclosure also includes another type of enhanced specificity Cas9 variant, “high fidelity” spCas9 variants (HF-Cas9). Examples of high fidelity variants include SpCas9-HF1 (N497A/R661A/Q695A/Q926A), SpCas9-HF2 (N497A/R661A/Q695A/Q926A/D1135E), SpCas9-HF3 (N497A/R661A/Q695A/Q926A/L169A), SpCas9-HF4 (N497A/R661A/Q695A/Q926A/Y450A). Also included are all SpCas9 variants bearing all possible single, double, triple and quadruple combinations of N497A, R661A, Q695A, Q926A or any other substitutions (Kleinstiver, B. P. et al., 2016, Nature. DOI: 10.1038/nature16526).

As used herein, the term “Cas” is meant to include all Cas molecules comprising variants, mutants, orthologs, high-fidelity variants and the like.

In one embodiment, the endonuclease is derived from a type II CRISPR/Cas system. In other embodiments, the endonuclease is derived from a Cas9 protein and includes Cas9, CasX, CasY.1, CasY.2, CasY.3, CasY.4, CasY.5, CasY.6, spCas, eSpCas, SpCas9-HF1, SpCas9-HF2, SpCas9-HF3, SpCas9-HF4, ARMAN 1, ARMAN 4, mutants, variants, high-fidelity variants, orthologs, analogs, fragments, or combinations thereof. The Cas9 protein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. Included are Cas9 proteins encoded in genomes of the nanoarchaea ARMAN-1 (Candidatus Micrarchaeum acidiphilum ARMAN-1) and ARMAN-4 (Candidatus Parvarchaeum acidiphilum ARMAN-4), CasY (Kerfeldbacteria, Vogelbacteria, Komeilibacteria, Katanobacteria), CasX (Planctomycetes, Deltaproteobacteria).

In general, CRISPR/Cas proteins comprise at least one RNA recognition and/or RNA binding domain. RNA recognition and/or RNA binding domains interact with guide RNAs. CRISPR/Cas proteins can also comprise nuclease domains (i.e., DNase or RNase domains), DNA binding domains, helicase domains, RNAse domains, protein-protein interaction domains, dimerization domains, as well as other domains. Active DNA-targeting CRISPR-Cas systems use 2 to 4 nucleotide protospacer-adjacent motifs (PAMs) located next to target sequences for self versus non-self discrimination. ARMAN-1 has a strong ‘NGG’ PAM preference. Cas9 also employs two separate transcripts, CRISPR RNA (crRNA) and trans-activating CRISPR RNA (tracrRNA), for RNA-guided DNA cleavage. Putative tracrRNA was identified in the vicinity of both ARMAN-1 and ARMAN-4 CRISPR-Cas9 systems.

Embodiments of the disclosure also include a new type of class 2 CRISPR-Cas system found in the genomes of two bacteria recovered from groundwater and sediment samples. This system includes Cas1, Cas2, Cas4 and an approximately 980 amino acid protein that is referred to as CasX. The high conservation (68% protein sequence identity) of this protein in two organisms belonging to different phyla, Deltaproteobacteria and Planctomycetes, suggests a recent cross-phyla transfer. The CRISPR arrays associated with each CasX has highly similar repeats (86% identity) of 37 nucleotides (nt), spacers of 33-34 nt, and a putative tracrRNA between the Cas operon and the CRISPR array. Distant homology detection and protein modeling identified a RuvC domain near the CasX C-terminal end, with organization reminiscent of that found in type V CRISPR-Cas systems. The rest of the CasX protein (630 N-terminal amino acids) showed no detectable similarity to any known protein, suggesting this is a novel class 2 effector. The combination of tracrRNA and separate Cas1, Cas2 and Cas4 proteins is unique among type V systems, and phylogenetic analyses indicate that the Cas 1 from the CRISPR-CasX system is distant from those of any other known type V. Further, CasX is considerably smaller than any known type V proteins: 980 aa compared to a typical size of about 1,200 amino acids for Cpf1, C2c1 and C2c3 (Burstein, D. et al., 2016 supra).

In some embodiments, a nucleic acid sequence of CCM2 comprises at least about a 50% sequence identity to wild type CCM2 or cDNA sequences thereof. In other embodiments, the CCM2 nucleic acid sequence comprises at least about 75%, 80%, 85%, 90%, 95%, 96%, 97%, 98%, 99% sequence identity to wild type CCM2 or cDNA sequences thereof, such as that set forth in SEQ ID NO: 1.

In some embodiments, a nucleic acid sequence of CCM2 further comprises one or more mutations, substitutions, deletions, variants or combinations thereof.

In some embodiments, the homology, sequence identity or complementarity, between a CCM2 nucleic acid sequence comprising one or more mutations, substitutions, deletions, variants or combinations thereof and the native or wild type or cDNA sequences of CCM2 is from about 50% to about 60%. In some embodiments, homology, sequence identity or complementarity, is from about 60% to about 70%. In some embodiments, homology, sequence identity or complementarity, is from about 70% to about 80%. In some embodiments, homology, sequence identity or complementarity, is from about 80% to about 90%. In some embodiments, homology, sequence identity or complementarity, is about 90%, about 92%, about 94%, about 95%, about 96%, about 97%, about 98%, about 99% or about 100%.

In those cases where the variants detected in a sample are predictive that a decrease of CCM2 levels or activity would be therapeutic, an agent capable of reducing expression or activity of CCM2 is administered to that subject.

In some embodiments, a nucleic acid sequence of TLNRD1 comprises at least about a 50% sequence identity to wild type TLNRD1 or cDNA sequences thereof. In other embodiments, the TLNRD1 nucleic acid sequence comprises at least about 75%, 80%, 85%, 90%, 95%, 96%, 97%, 98%, 99% sequence identity to wild type TLNRD1 or cDNA sequences thereof, such as that set forth in SEQ ID NO: 2.

In some embodiments, a nucleic acid sequence of TLNRD1 further comprises one or more mutations, substitutions, deletions, variants or combinations thereof.

In some embodiments, the homology, sequence identity or complementarity, between a TLNRD1 nucleic acid sequence comprising one or more mutations, substitutions, deletions, variants or combinations thereof and the native or wild type or cDNA sequences of TLNRD1 is from about 50% to about 60%. In some embodiments, homology, sequence identity or complementarity, is from about 60% to about 70%. In some embodiments, homology, sequence identity or complementarity, is from about 70% to about 80%. In some embodiments, homology, sequence identity or complementarity, is from about 80% to about 90%. In some embodiments, homology, sequence identity or complementarity, is about 90%, about 92%, about 94%, about 95%, about 96%, about 97%, about 98%, about 99% or about 100%.

In those cases where the variants detected in a sample are predictive that a decrease of TLNRD1 levels or activity would be therapeutic, an agent capable of reducing expression or activity of TLNRD1 is administered to that subject.

Suitable nucleic acid delivery systems include viral vector, typically sequence from at least one of an adenovirus, adenovirus-associated virus (AAV), helper-dependent adenovirus, retrovirus, or hemagglutinating virus of Japan-liposome (HVJ) complex. In a particular example, the viral vector comprises a strong eukaryotic promoter operably linked to the polynucleotide e.g., a cytomegalovirus (CMV) promoter. In another example, the viral vector comprises a truncated base-editing system such as that described in Davis et al., Nature Biomedical Engineering, doi.org/10.1038/s41551-022-000911-4 that has been suitably modified to target and correct CCM2 variants. In certain embodiments, the viral capsid has been modified for enhanced transduction of human cardiomyocytes.

If desired, the polynucleotides of the disclosure may also be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Feigner and Holm, Bethesda Res. Lab. Focus, 11 (2): 21 (1989) and Maurer, R. A., Bethesda Res. Lab. Focus, 11 (2): 25 (1989).

Replication-defective recombinant adenoviral vectors, can be produced in accordance with known techniques. See, Quantin, et al., Proc. Natl. Acad. Sci. USA, 89:2581-2584 (1992); Stratford-Perricadet, et al., J. Clin. Invest., 90:626-630 (1992); and Rosenfeld, et al., Cell, 68:143-155 (1992).

One exemplary delivery system is a recombinant viral vector that incorporates one or more of the polynucleotides therein, for example, about one polynucleotide. An exemplary viral vector used in the disclosed methods has a pfu (plague forming units) of from about 10⁸ to about 5×10¹⁰ pfu. In embodiments in which the polynucleotide is to be administered with a non-viral vector, use of between from about 0.1 nanograms to about 4000 micrograms will often be useful e.g., about 1 nanogram to about 100 micrograms.

In some embodiments, the vector is an adenovirus-associated viral vector (AAV), for example, AAV9. The term “AAV vector” means a vector derived from an adeno-associated virus serotype, including without limitation, AAV-1, AAV-2, AAV-3, AAV-4, AAV-5, AAV-6, AAV-7 and AAV-8. AAV vectors can have one or more of the AAV wild-type genes deleted in whole or part, such as the rep and/or cap genes, but retain functional flanking ITR sequences. Despite the high degree of homology, the different serotypes have tropisms for different tissues. The receptor for AAV1 is unknown; however, AAV1 is known to transduce skeletal and cardiac muscle more efficiently than AAV2. Since most of the studies have been done with pseudotyped vectors in which the vector DNA flanked with AAV2 ITR is packaged into capsids of alternate serotypes, the biological differences may be related to the capsid rather than to the genomes. Recent evidence indicates that DNA expression cassettes packaged in AAV 1 capsids are at least 1 log 10 more efficient at transducing cardiomyocytes than those packaged in AAV2 capsids. In one embodiment, the viral delivery system is an adeno-associated viral delivery system. The adeno-associated virus can be of serotype 1 (AAV1), serotype 2 (AAV2), serotype 3 (AAV3), serotype 4 (AAV4), serotype 5 (AAV5), serotype 6 (AAV6), serotype 7 (AAV7), serotype 8 (AAV8), or serotype 9 (AAV9).

Some skilled in the art have circumvented some of the limitations of adenovirus-based vectors by using adenovirus “hybrid” viruses, which incorporate desirable features from adenovirus as well as from other types of viruses as a means of generating unique vectors with highly specialized properties. For example, viral vector chimeras were generated between adenovirus and adeno-associated virus (AAV). These aspects of the disclosure do not deviate from the scope of the disclosure described herein.

Nucleic acids encoding the gene editing systems of the disclosure may be delivered to arterial endothelial cells by methods known in the art. For example, arterial endothelial cells of a large mammal may be transfected by a method that includes dilating a blood vessel of the coronary circulation by administering a vasodilating substance to said mammal prior to, and/or concurrently with, administering the nucleic acids. In some embodiments, the method includes administering the nucleic acids into a blood vessel of the coronary circulation in vivo, wherein nucleic acids are infused into the blood vessel over a period of at least about three minutes, wherein the coronary circulation is not isolated or substantially isolated from the systemic circulation of the mammal, and wherein the nucleic acids transfect cardiac cells of the mammal.

In some embodiments, the subject can be a human, an experimental animal, e.g., a rat or a mouse, a domestic animal, e.g., a dog, cow, sheep, pig or horse, or a non-human primate, e.g., a monkey. The subject may be suffering from a vascular disease, such as CAD. In some embodiments, the subject is suffering from CAD. In some embodiments, the subject is a human. For example, the subject is a human between ages 18 and 65. In another embodiment, the subject is a non-human animal.

In some embodiments, the subject has or is at risk for vascular disease, e.g. coronary artery disease (CAD).

In some embodiments, transfection of endothelial cells (e.g., arterial endothelial cells) with nucleic acid molecules encoding a gene editing system described herein decreases complications associated with CAD, including but not limited to clot formation, plaque build-up (e.g., atherosclerosclerotic plaque build-up), arterial calcification, arterial inflammation, and risk of thrombosis.

A treatment can be evaluated by assessing the effect of the treatment on a parameter related to contractility. For example, SR Ca.sup.2+ ATPase activity or intracellular Ca.sup.2+ concentration can be measured. Furthermore, force generation by hearts or heart tissue can be measured using methods described in Strauss et al., Am. J. Physiol., 262:1437-45, 1992, the contents of which are incorporated herein by reference.

Modified Nucleic Acid Sequences: It is not intended that the disclosure be limited by the nature of the nucleic acid employed. The nucleic acid may be DNA or RNA and may exist in a double-stranded, single-stranded or partially double-stranded form.

Nucleic acids useful in the disclosure include, by way of example and not limitation, oligonucleotides and polynucleotides such as antisense DNAs and/or RNAs; ribozymes; sgRNAs; inhibitory nucleic acids; DNA for gene therapy; viral fragments including viral DNA and/or RNA; DNA and/or RNA chimeras; mRNA; plasmids; cosmids; genomic DNA; cDNA; gene fragments; various structural forms of DNA including single-stranded DNA, double-stranded DNA, supercoiled DNA and/or triple-helical DNA; Z-DNA; and the like. The nucleic acids may be prepared by any conventional means typically used to prepare nucleic acids in large quantities. For example, DNAs and RNAs may be chemically synthesized using commercially available reagents and synthesizers by methods that are well-known in the art (see, e.g., Gait, 1985, Oligonucleotide Synthesis: A Practical Approach (IRL Press, Oxford, England)). RNAs may be produced in high yield via in vitro transcription using plasmids such as pGEM™ T vector or SP65 (Promega Corporation, Madison, Wis.).

Accordingly, certain nucleic acid sequences of this disclosure are chimeric nucleic acid sequences. “Chimeric nucleic acid sequences” or “chimeras,” in the context of this disclosure, contain two or more chemically distinct regions, each made up of at least one nucleotide. These sequences typically contain at least one region of modified nucleotides that confers one or more beneficial properties (such as, for example, increased nuclease resistance, increased uptake into cells, increased binding affinity for the target).

Chimeric nucleic acid sequences of the disclosure may be formed as composite structures of two or more oligonucleotides, modified oligonucleotides, oligonucleotides and/or oligonucleotide mimetics. Such compounds have also been referred to in the art as hybrids or gapmers. Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

Specific examples of some modified nucleic acid sequences envisioned for this disclosure include those comprising modified backbones, for example, phosphorothioates, phosphotriesters, methyl phosphonates, short chain alkyl or cycloalkyl intersugar linkages or short chain heteroatomic or heterocyclic intersugar linkages. In some embodiments, modified oligonucleotides comprise those with phosphorothioate backbones and those with heteroatom backbones, CH₂—NH—O—CH₂, CH, —N(CH₃)—O—CH₂ [known as a methylene(methylimino) or MMI backbone], CH₂—O—N(CH₃)—CH₂2, CH₂—N(CH₃)—N(CH₃)—CH₂ and O—N(CH₃)—CH₂—CH₂ backbones, wherein the native phosphodiester backbone is represented as O—P—O—CH).

The amide backbones disclosed by De Mesmaeker et al. Ace. Chem. Res. 1995, 28:366-374) are also embodied herein. In some embodiments, the nucleic acid sequences having morpholino backbone structures (Summerton and Weller, U.S. Pat. No. 5,034,506), peptide nucleic acid (PNA) backbone wherein the phosphodiester backbone of the oligonucleotide is replaced with a polyamide backbone, the nucleobases being bound directly or indirectly to the aza nitrogen atoms of the polyamide backbone (Nielsen et al. Science 1991, 254, 1497). The nucleic acid sequences may also comprise one or more substituted sugar moieties. The nucleic acid sequences may also have sugar mimetics such as cyclobutyls in place of the pentofuranosyl group.

Exemplary modified oligonucleotide backbones comprise, but are not limited to, phosphorothioates, chiral phosphorothioates, phosphorodithioates, phosphotriesters, aminoalkylphosphotriesters, methyl and other alkyl phosphonates comprising 3′ alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Exemplary modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

The nucleic acid sequences may also include, additionally or alternatively, nucleobase (often referred to in the art simply as “base”) modifications or substitutions. As used herein, “unmodified” or “natural” nucleotides include adenine (A), guanine (G), thymine (T), cytosine (C) and uracil (U). Modified nucleotides include nucleotides found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentiobiosyl HMC, as well as synthetic nucleotides, e.g., 2-aminoadenine, 2-(methylamino) adenine, 2-(imidazolylalkyl) adenine, 2-(aminoalkylamino) adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl) adenine and 2,6-diaminopurine. (Kornberg, A., DNA Replication, W.H. Freeman & Co., San Francisco, 1980, pp 75-77; Gebeyehu, G., (1987) et al. Nucl. Acids Res. 15:4513). A “universal” base known in the art, e.g., inosine, may be included.

Another modification involves chemically linking to the oligonucleotide one or more moieties or conjugates which enhance the activity or cellular uptake of the oligonucleotide. Such moieties include but are not limited to lipid moieties such as a cholesterol moiety, a cholesteryl moiety, cholic acid, a thioether, e.g., hexyl-5-tritylthiol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid. Nucleic acid sequences comprising lipophilic moieties, and methods for preparing such oligonucleotides are known in the art, for example, U.S. Pat. Nos. 5,138,045, 5,218,105 and 5,459,255.

It is not necessary for all positions in a given nucleic acid sequence to be uniformly modified, and in fact more than one of the aforementioned modifications may be incorporated in a single nucleic acid sequence or even at a single nucleotide within such sequences. The disclosure also includes oligonucleotides which are chimeric oligonucleotides as hereinbefore defined.

In another embodiment, the nucleic acid molecule of the disclosure is conjugated with another moiety including but not limited to basic nucleotides, polyether, polyamine, polyamides, peptides, carbohydrates, lipid, or polyhydrocarbon compounds. Those skilled in the art will recognize that these molecules can be linked to one or more of any nucleotides comprising the nucleic acid molecule at several positions on the sugar, base or phosphate group.

In another embodiment, the nucleic acid sequences comprise one or more nucleotides substituted with locked nucleic acids (LNA). The LNA modified nucleic acid sequences may have a size similar to the parent or native sequence or may be larger or smaller. Such LNA-modified oligonucleotides may contain less than about 70%, or less than about 60%, or less than about 50% LNA monomers and that their sizes are between about 1 and 25 nucleotides.

### Subject Being Treated

A subject being treated for vascular disease (e.g., CAD) according to the disclosed methods and uses may exemplify one or more of the underlying gene expression patterns that are disclosed herein. In particular, a subject with or at risk for vascular disease (e.g., CAD) that is to be treated according to the disclosed methods and uses may express a polymorphism in one or more genes, such as one or more genes of the CCM pathway, one or more genes that regulate the CCM pathway, or a combination thereof. In some embodiments, a subject with or at risk for vascular disease (e.g., CAD) that is to be treated according to the disclosed methods and uses may express a polymorphism in one or more genes selected from the group consisting of TLNRD1, CCM2, HEG1, ITGB1BP1, KRIT1, PDCD10, ARPC2, CDC42, CDH5, DNM2, MEAF6, PDCD7, RHOA, KLF2, KLF4, MAP2K5, MAP3K3, MEF2A, and NFAT5. In some embodiments, a subject with or at risk for vascular disease may express a loss-of-function variant of CCM2, TLNRD1, CCM2, HEG1, ITGB1BP1, KRIT1, PDCD10, ARPC2, CDC42, CDH5, DNM2, MEAF6, PDCD7, RHOA, KLF2, KLF4, MAP2K5, MAP3K3, MEF2A, or NFAT5. A subject may be heterozygous or homozygous for a variant. A polymorphism or variant may be observed in a sample or test sample obtained from a subject.

A subject may be a human individual of any race or gender.

In some embodiments, the subject has been diagnosed with a vascular disease (e.g., CAD) for at least about 1 month, at least about 2 months, at least about 3 months, at least about 4 months, at least about 5 months, at least about 6 months, or at least about 1 year or more.

In some embodiments, the subject has not been previously diagnosed with a vascular disease (e.g., CAD). In some embodiments, the subject does not have symptoms of vascular disease (e.g., CAD).

### Doses and Dosing Regimen for the Disclosed Methods and Uses

An effective amount of one or more pharmacological agents can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present disclosure for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment and prevention dosages generally may be titrated to optimize safety and efficacy. The dosage can be determined by a physician and adjusted, as necessary, to suit observed effects of the treatment. In some cases, dosage-effect relationships from in vitro and/or in vivo tests initially can provide useful guidance on the proper doses for patient administration. In general, one will desire to administer an amount of the agent that is effective to achieve a serum level commensurate with the concentrations found to be effective in vitro. Determination of these parameters is well within the skill of the art. These considerations, as well as effective formulations and administration procedures are well known in the art and are described in standard textbooks.

Dosage regimens for treating or preventing vascular disease (e.g., CAD) may comprise flat dosing (i.e., administering the same dose repeatedly at pre-determined intervals) or comprise a loading dose (i.e., administering an initial dose that is higher or different than subsequent, serial doses). For the purposes of either type of dosing regimen an effective dose may be administered topically, parenterally, subcutaneously, subdermally, intradermally, or intramuscularly.

In some embodiments, a loading dose and the subsequent serial doses may be administered via the same route (e.g., subcutaneously), while in some embodiments, a loading dose and the subsequent serial doses may be administered via different routes (e.g., parenterally and subcutaneously, respectively). In some embodiments, the loading dose is administered as a single injection. In some embodiments, the loading dose is administered as multiple injections, which may be administered at the same time or spaced apart at defined intervals. The subsequent serial doses of a loading dose regimen are generally lower than the loading dose.

In some embodiments of the disclosed methods and uses, the duration of treatment or prevention is about one day, about one week, about two weeks, about three weeks, about four weeks, about five weeks, about six weeks, about seven weeks, about eight weeks, about nine weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 24 weeks, about 30 weeks, about 36 weeks, about 40 weeks, about 48 weeks, about 50 weeks, about one year, about two years, about three years, about four years, about five years, or as needed based on the appearance of symptoms of the vascular disease (e.g., CAD).

The present disclosure provides uses of a therapy described herein in the manufacture of a medicament for the treatment or prevention of a vascular disease, such as CAD, for disrupting the CCM pathway in subjects with or at risk for vascular disease (e.g., CAD), and/or for normalizing vascular or cardiovascular function. All of the disclosed doses, dosing regimens, routes of administrations, biomarkers, and therapeutic endpoints are applicable to these uses as well.

Routes and frequency of administration of the therapeutic agents disclosed herein, as well as dosage, will vary from individual to individual as well as with the selected drug, and can be readily established using standard techniques. In general, the pharmaceutical compositions can be administered, by injection (e.g., intracutaneous, intramuscular, intravenous or subcutaneous), intranasally (e.g., by aspiration) or orally.

Dosage forms typically include an “excipient,” which as used herein, is any component of a dosage form that is not an API. Excipients include binders, lubricants, diluents, disintegrants, coatings, barrier layer components, glidants, and other components. Excipients are known in the art (see HANDBOOK OF PHARMACEUTICAL EXCIPIENTS, FIFTH EDITION, 2005, edited by Rowe et al., McGraw Hill). Some excipients serve multiple functions or are so-called high functionality excipients. For example, talc can act as a lubricant, and an anti-adherent, and a glidant. See Pifferi et al., 2005, “Quality and functionality of excipients” Farmaco. 54:1-14; and Zeleznik and Renak, Business Briefing: Pharmagenerics 2004.

A suitable dose is an amount of a compound that, when administered as described above, is capable of promoting an anti-cardiac disease therapeutic response. Such response can be monitored using conventional methods. In general, for pharmaceutical compositions, the amount of each drug present in a dose ranges from about 100 μg to 5 mg per kg of host, but those skilled in the art will appreciate that specific doses depend on the drug to be administered and are not necessarily limited to this general range. Likewise, suitable volumes for each administration will vary with the size of the patient.

In the context of treatment, a “therapeutically effective amount” of a drug is an amount of or its pharmaceutically acceptable salt which eliminates, alleviates, or provides relief of the symptoms for which it is administered. The disclosed compositions are administered in any suitable manner, often with pharmaceutically acceptable carriers. Suitable methods of administering treatment in the context of the disclosure to a subject are available, and, although more than one route can be used to administer a particular composition, a particular route can often provide a more immediate and more effective reaction than another route. The dose administered to a patient, in the context of the disclosure, should be sufficient to effect a beneficial therapeutic response in the patient over time, or to inhibit disease progression. Thus, the composition is administered to a subject in an amount sufficient to elicit an effective response and/or to alleviate, reduce, cure or at least partially arrest symptoms and/or complications from the disease. An amount adequate to accomplish this is defined as a “therapeutically effective dose.”

In general, an appropriate dosage and treatment regimen involves administration of the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients.

### Methods of Prognosis

Methods of treating, predicting, or preventing vascular diseases are also provided herein and can be used in combination with the diagnostic and prognostic method provided for detection and monitoring of the CCM pathway associated genes described herein. In some embodiments, methods for detecting mutations in CCM pathway-associated genes are provided for treating, predicting, diagnosing, providing prognosis, determining the likelihood that a patient will respond to a therapy, and/or managing a disease or condition related to a vascular disease or disorder. In some embodiments, methods for detecting CCM pathway associated gene mutations are provided for treating or preventing a vascular disease or condition, in conjunction with one or more therapies effective in treating the disease or condition, including but not limited to drug therapy and gene therapy.

Provided herein, in certain embodiments, are methods for determining the prognosis of a patient having a vascular disease (e.g., CAD), comprising: performing a nucleic acid detection assay on a sample comprising nucleic acids from a patient to determine whether the nucleic acid comprises a mutation, for example, where the mutation is loss-of-function mutation or a nonsense (stop-gain) mutation; and diagnosing the patient as having a favorable prognosis when the mutation is detected.

The phrase “determining the prognosis” as used herein refers to the process by which the skilled artisan can predict the course or outcome of a condition in a patient. The term “prognosis” does not refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. A prognosis can be expressed as the amount of time a patient can be expected to survive. Alternatively, a prognosis can refer to the likelihood that the disease goes into remission or to the amount of time the disease can be expected to remain in remission. Prognosis can be expressed in various ways; for example prognosis can be expressed as a percent chance that a patient will survive after one year, five years, ten years or the like. Alternatively prognosis can be expressed as the number of years, on average that a patient can expect to survive as a result of a condition or disease. The prognosis of a patient can be considered as an expression of relativism, with many factors effecting the ultimate outcome. For example, for patients with certain conditions, prognosis can be appropriately expressed as the likelihood that a condition can be treatable or curable, or the likelihood that a disease will go into remission, whereas for patients with more severe conditions prognosis can be more appropriately expressed as likelihood of survival for a specified period of time. In certain embodiments, a prognosis can be expressed as the likelihood that a subject or patient who is positive for a mutation in a particular gene, but who does not present with a particular disease (e.g., a disease linked to or associated with the gene), will subsequently develop the particular disease.

The term “favorable prognosis” as used herein, in the context of a patient having a vascular disease and a mutation in CCM pathway gene or CCM pathway associated gene (i.e., a gene that regulates the CCM pathway, or a gene that is a member of the CCM pathway), refers to an increased likelihood that the patient will have a better outcome in a clinical condition relative to a patient diagnosed as having the same disease but without the mutation. A favorable prognosis can be expressed in any relevant prognostic terms and can include, for example, the expectation of an increased duration of remission, increased survival rate, and increased survival duration.

Provided herein is a method for prognosing a subject having or suspected of having a vascular disease (e.g., CAD), comprising: (a) detecting in a sample obtained from the subject the presence or absence of one or more nucleic acid sequence variant that results in loss-of-function of one or more genes of the Cerebral Cavernous Malformation (CCM) pathway or one or more genes that regulate the CCM pathway; and (b) prognosing the subject as having a favorable prognosis if the sequence variant is detected. In some embodiments, the sequence variant is a sequence variant of one or more of TLNRD1, CCM2, HEG1, ITGB1BP1, KRIT1, PDCD10, ARPC2, CDC42, CDH5, DNM2, MEAF6, PDCD7, RHOA, KLF2, KLF4, MAP2K5, MAP3K3, MEF2A, and NFAT5.

Provided herein is a method for prognosing a subject having or suspected of having a vascular disease (e.g., CAD), comprising: (a) detecting in a sample obtained from the subject the presence or absence of one or more nucleic acid sequence variant that results in loss-of-function of one or more target genes; and (b) prognosing the subject as having a favorable prognosis if the sequence variant is detected. In some embodiments, the target gene is selected from the group consisting of TLNRD1, CCM2, HEG1, ITGB1BP1, KRIT1, PDCD10, ARPC2, CDC42, CDH5, DNM2, MEAF6, PDCD7, RHOA, KLF2, KLF4, MAP2K5, MAP3K3, MEF2A, and NFAT5.

Provided herein, in certain embodiments, are methods for determining the likelihood that a patient will respond to a therapy. In some embodiments, provided is a method for determining whether a subject having or suspected of having a vascular disease (e.g., CAD) is likely to respond to a therapy, comprising: (a) detecting in a sample obtained from the subject the presence or absence of one or more nucleic acid sequence variant that results in loss-of-function of one or more genes of the Cerebral Cavernous Malformation (CCM) pathway or one or more genes that regulate the CCM pathway (i.e., target genes); and (b) determining that the subject is more likely to respond to the therapy if the sequence variant is detected. In some embodiments, the sequence variant is a sequence variant of one or more of TLNRD1, CCM2, HEG1, ITGB1BP1, KRIT1, PDCD10, ARPC2, CDC42, CDH5, DNM2, MEAF6, PDCD7, RHOA, KLF2, KLF4, MAP2K5, MAP3K3, MEF2A, and NFAT5.

Provided herein, in certain embodiments, are methods for determining the likelihood that a patient will respond to a therapy. In some embodiments, provided is a method for determining whether a subject having or suspected of having a vascular disease (e.g., CAD) is likely to respond to a therapy, comprising: (a) detecting in a sample obtained from the subject the presence or absence of one or more nucleic acid sequence variant that results in loss-of-function of one or more target genes; and (b) determining that the subject is more likely to respond to the therapy if the sequence variant is detected. In some embodiments, the target gene is selected from the group consisting of TLNRD1, CCM2, HEG1, ITGB1BP1, KRIT1, PDCD10, ARPC2, CDC42, CDH5, DNM2, MEAF6, PDCD7, RHOA, KLF2, KLF4, MAP2K5, MAP3K3, MEF2A, and NFAT5.

Provided is a method for determining whether a subject having, suspected of having, or at risk for a vascular disease is likely to respond to a therapy for the vascular disease, comprising detecting in a sample obtained from the subject the presence or absence of one or more nucleic acid sequence variant that results in loss-of-function of one or more genes of the Cerebral Cavernous Malformation (CCM) pathway or one or more genes that regulate the CCM pathway. In some embodiments, the sample is a biological sample comprising nucleic acids.

In some embodiments, a therapy comprises administering to the subject a therapeutically effective amount of a pharmacological agent capable of modulating the expression of a target gene in vascular endothelial cells. In some embodiments, the target gene comprises a Cerebral Cavernous Malformation (CCM) pathway gene and/or a gene that regulates the function of the CCM pathway. In some embodiments, the target gene is selected from the group consisting of TLNRD1, CCM2, HEG1, ITGB1BP1, KRIT1, PDCD10, ARPC2, CDC42, CDH5, DNM2, MEAF6, PDCD7, RHOA, KLF2, KLF4, MAP2K5, MAP3K3, MEF2A, and NFAT5. In some embodiments, the therapy comprises a CRISPR therapy. In some embodiments, the pharmacological agent is a gene editing system. In some embodiments, the gene editing system comprises a Clustered Regularly Interspaced Short Palindromic Repeats (CRISPR)-associated (CRISPR-Cas) system, comprising (a) a single guide RNA (sgRNA) which comprises a guide sequence capable of hybridizing with the target sequence, or a polynucleotide encoding the sgRNA, and (b) an effector protein, or one or more nucleotide sequences encoding the effector protein; wherein the sgRNA hybridizes to the target sequence, and the sgRNA forms a complex with the effector protein; and wherein the effector protein comprises a nuclease and/or an effector domain. In some embodiments, the guide sequence is linked to a direct repeat sequence. In some embodiments, the sgRNA is capable of hybridizing to a target gene, for example, one or more Cerebral Cavernous Malformation (CCM) pathway gene and/or one or more genes that regulate the function of the CCM pathway. In some embodiments, the sgRNA is capable of hybridizing to one or more genes selected from the group consisting of: TLNRD1, CCM2, HEG1, ITGB1BP1, KRIT1, PDCD10, ARPC2, CDC42, CDH5, DNM2, MEAF6, PDCD7, RHOA, KLF2 KLF4, MAP2K5, MAP3K3, MEF2A, and NFAT5. In some embodiments, the sgRNA comprises a nucleotide sequence as set forth in any one of SEQ ID NO: 3-209. In some embodiments, the sgRNA comprises a nucleotide sequence as set forth in any one of SEQ ID NO: 4, 14, 48, 49, 84, 91, 93, 94, 129, 139, 200, 201, 202, 203, 204, 205, 206, 207, 208, and 209.

Provided is a method for determining whether a subject having, suspected of having, or at risk for a vascular disease is likely to respond to a therapy for the vascular disease, comprising: (a) analyzing a biological sample obtained from the subject, wherein the biological sample comprises nucleic acids; (b) detecting the presence or absence of one or more nucleic acid sequence variant that results in loss-of-function of one or more genes of the Cerebral Cavernous Malformation (CCM) pathway or one or more genes that regulate the CCM pathway (i.e., target genes); and (c) determining that the subject is more likely to respond to the therapy if the sequence variant is detected. In some embodiments, the method further comprises (d) determining that the subject is less likely to respond to the therapy if the sequence variant is not detected.

### VII. Cells, Vectors, and Polynucleotides

In some aspects, the present disclosure provides an engineered, non-naturally occurring vector system comprising (a) one or more vectors comprising a first regulatory element operably linked to the present guide RNA (e.g., sgRNAs described herein) that targets a DNA molecule encoding a gene product and (b) a second regulatory element operably linked to an effector protein. Components (a) and (b) may be located on the same or different vectors of the system. The present guide RNA targets the DNA molecule encoding the gene product in a cell and the effector protein modifies the expression of the DNA molecule encoding the gene product; and, wherein the effector protein and the guide RNA do not naturally occur together.

In some embodiments, component (a) of the above vectors further comprises two or more guide sequences operably linked to the first regulatory element, wherein when expressed, each of the two or more guide sequences direct sequence specific binding of an effector protein complex (e.g., a CRISPR complex) to a different target sequence in a eukaryotic cell.

In some aspects, the present disclosure provides a vector system comprising one or more vectors, wherein the one or more vectors comprises: (a) a first expression regulatory element operably linked to a nucleotide sequence encoding an effector protein, or one or more nucleotide sequences encoding the effector protein; and (b) a second expression regulatory element operably linked to one or more nucleotide sequences encoding a single guide RNA (sgRNA) comprising a guide sequence capable of hybridizing to a target sequence, wherein components (a) and (b) are located on the same or different vectors. In some embodiments, the sgRNA is capable of hybridizing to one or more genes of the Cerebral Cavernous Malformation (CCM) pathway and/or one or more genes that regulate the CCM pathway (i.e., target genes).

In some embodiments, guide RNA forms a complex with the effector protein to form an effector protein complex. For example, a guide RNA can form a complex with a CRISPR enzyme to form a CRISPR complex. The effector protein complex or polynucleotides encoding it may comprise one or more nuclear localization sequences of sufficient strength to drive accumulation of said effector protein complex in a detectable amount in the nucleus of a eukaryotic cell.

In some embodiments, the effector protein is a CRISPR enzyme. In some embodiments, the CRISPR enzyme is a Cas9 enzyme. In some embodiments, the Cas9 enzyme is S. aureus, S. pneumoniae, S. pyogenes, or S. thermophilus Cas9, and may include mutated Cas9 derived from these organisms. The enzyme may be a Cas9 homolog or ortholog. In some embodiments, the CRISPR enzyme is codon-optimized for expression in a eukaryotic cell. In some embodiments, the CRISPR enzyme directs cleavage of one or two strands at the location of the target sequence.

In some embodiments, the first regulatory element in the present vectors is a polymerase III promoter. In some embodiments, the second regulatory element in the present vectors is a polymerase II promoter.

In some embodiments, the guide sequence is at least 15, 16, 17, 18, 19, 20, 25 nucleotides, or between 10-30, or between 15-25, or between 20-24 nucleotides in length.

In any of the sgRNA, DNA polynucleotide molecule, DNA expression vector, delivery vector, method, system, composition or complex described herein said manipulation may be performed in vitro or ex vivo.

In one aspect, the present disclosure also provides any of the sgRNA, DNA polynucleotide molecule, DNA expression vector, delivery vector, system, composition or complex as described herein for use as a medicament, optionally for use in the treatment of vascular disease. In such cases, treatment is treatment of a mammal, such as a human.

In one aspect, the present disclosure also provides pharmaceutical compositions comprising any of the sgRNAs, DNA polynucleotide molecules, DNA expression vectors, delivery vectors, systems, compositions or complexes described herein. Such pharmaceutical compositions may contain an excipient. Such pharmaceutical compositions may be formulated for administration to a mammal, such as a human.

It will be appreciated that the disclosure described herein involves various components which may display variations in their specific characteristics. It will be appreciated that any combination of features described above and herein, as appropriate, are contemplated as a means for implementing the disclosure.

In general, applying to any of the aspects discussed herein, the sgRNA is a non-naturally occurring single guide RNA molecule. It is capable of effecting the manipulation of a target nucleic acid within a prokaryotic or eukaryotic cell when in complex within the cell with an effector protein (e.g., a CRISPR enzyme). Examples of CRISPR enzymes are a Staphylococcus aureus Cas9 enzyme (SaCas9) or other similar smaller Cas9 orthologs. The sgRNA may comprise, in some embodiments, the following, in any tandem arrangement:
I. a guide sequence, which is capable of hybridizing to a sequence of the target nucleic acid to be manipulated;
II. a tracr mate sequence, comprising a region of sense sequence;
III. a linker sequence; and
IV. a tracr sequence, comprising a region of antisense sequence which is positioned adjacent the linker sequence and which is capable of hybridizing with the region of sense sequence thereby forming a stem loop.

In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the disclosure in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell).

The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoters (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8 (1), p. 466-472, 1988); SV40 enhancer; and the intron sequence between exons 2 and 3 of rabbit β-globin (Proc. Natl. Acad. Sci. USA., Vol. 78 (3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

Advantageous vectors include lentiviruses and adeno-associated viruses (AAV), and types of such vectors can also be selected for targeting particular types of cells.

In one aspect, the disclosure provides a eukaryotic host cell. The host cell may comprise (a) a first expression regulatory element operably linked to a nucleotide sequence encoding an effector protein, or one or more nucleotide sequences encoding the effector protein; and (b) a second expression regulatory element operably linked to one or more nucleotide sequences encoding a single guide RNA (sgRNA) comprising a guide sequence capable of hybridizing to a target sequence. In some embodiments, the host cell comprises components (a) and (b). In some embodiments, component (a), component (b), or components (a) and (b) are stably integrated into a genome of the host eukaryotic cell. In some embodiments, component (a) further comprises the tracr sequence downstream of the tracr mate sequence under the control of the first regulatory element. In some embodiments, component (b) further comprises two or more guide sequences operably linked to the first regulatory element, wherein when expressed, each of the two or more guide sequences direct sequence specific binding of an effector protein complex (e.g., a CRISPR complex) to a different target sequence in a eukaryotic cell. The effector protein may be an enzyme, such as a CRISPR enzyme (e.g., a Cas9 homolog or ortholog). In some embodiments, the CRISPR enzyme is codon-optimized for expression in a eukaryotic cell. In some embodiments, the CRISPR enzyme directs cleavage of one or two strands at the location of the target sequence. In some embodiments, the CRISPR enzyme lacks DNA strand cleavage activity. In some embodiments, the guide sequence is at least 15, 16, 17, 18, 19, 20, 25 nucleotides, or between 10-30, or between 15-25, or between 15-20 nucleotides in length. In one aspect, the disclosure provides a non-human eukaryotic organism, such as a multicellular eukaryotic organism, comprising a eukaryotic host cell according to any of the described embodiments. In other aspects, the disclosure provides a eukaryotic organism, such as multicellular eukaryotic organism, comprising a eukaryotic host cell according to any of the described embodiments. The organism in some embodiments of these aspects may be an animal, for example a mammal.

With respect to use of the CRISPR-Cas system generally, mention is made of the documents, including patent applications, patents, and patent publications cited throughout this disclosure as embodiments of the disclosure can be used as in those documents.

In one aspect, the present disclosure provides a vector system or eukaryotic host cell comprising (a) a first expression regulatory element operably linked to a nucleotide sequence encoding an effector protein, or one or more nucleotide sequences encoding the effector protein; and (b) a second expression regulatory element operably linked to one or more nucleotide sequences encoding a single guide RNA (sgRNA) comprising a guide sequence capable of hybridizing to a target sequence. Components (a) and (b) can be on the same or different vectors. In some embodiments, the sgRNA is capable of hybridizing to one or more genes of the Cerebral Cavernous Malformation (CCM) pathway or one or more genes that regulate the CCM pathway (i.e., target genes). In some embodiments, the one or more genes are selected from the group consisting of: TLNRD1, CCM2, HEG1, ITGB1BP1, KRIT1, PDCD10, ARPC2, CDC42, CDH5, DNM2, MEAF6, PDCD7, RHOA, KLF2, KLF4, MAP2K5, MAP3K3, MEF2A, and NFAT5. In some embodiments, the host cell comprises components (a) and (b). In some embodiments, component (a), component (b), or components (a) and (b) are stably integrated into a genome of the host eukaryotic cell.

### VIII. Kits

The present disclosure also contemplates detection, diagnostic, prognostic, and treatment systems in kit form. In some embodiments, a kit can be used for conducting the diagnostic, prognostic, or treatment methods described herein. In some embodiments, a kit can be used as a companion diagnostic for detection of a CCM pathway-associated gene variant in a subject that has received one or more treatments for a vascular disease or condition (e.g., CAD), or that has not received any treatments for a vascular disease or condition.

The kit may contain, in a carrier or compartmentalized container, reagents useful in any of the above-described embodiments of the methods.

A detection system provided herein can include a kit that contains, in an amount sufficient for at least one assay, any of the hybridization assay probes and amplification primers for detection of nucleic acids encoding a CCM pathway associated gene or loss-of-function variant thereof, such as those discussed herein. For example, a kit provided herein can contain in an amount sufficient for at least one assay, any of the hybridization assay probes and amplification primers for detection of nucleic acids encoding a CCM pathway associated gene or loss-of-function variant thereof, such as, without limitation, one or more of TLNRD1, CCM2, HEG1, ITGB1BP1, KRIT1, PDCD10, ARPC2, CDC42, CDH5, DNM2, MEAF6, PDCD7, RHOA, KLF2, KLF4, MAP2K5, MAP3K3, MEF2A, and NFAT5.

In some embodiments, the kit includes one or more primers or probes suitable for amplification and/or sequencing. The primers can be labeled with a detectable marker such as radioactive isotopes, or fluorescence markers.

In some embodiments, the kit may also include primers for the amplification of one or more housekeeping genes. Non-limiting examples of housekeeping genes include GAPDH, ACTB, TUBB, UBQ, PGK, and RPL.

Typically, the kits will also include instructions recorded in a tangible form (e.g., contained on paper or an electronic medium) for using the packaged probes, primers, and/or antibodies in a detection assay for determining the presence or amount of mutant nucleic acid or protein in a test sample.

The various components of the detection, diagnostic, prognostic, and treatment systems can be provided in a variety of forms. For example, the required enzymes, the nucleotide triphosphates, the probes, primers, and/or antibodies can be provided as a lyophilized reagent. These lyophilized reagents can be pre-mixed before lyophilization so that when reconstituted they form a complete mixture with the proper ratio of each of the components ready for use in the assay. In addition, the systems of the disclosures can contain a reconstitution reagent for reconstituting the lyophilized reagents of the kit. In an exemplary kit, the enzymes, nucleotide triphosphates and required cofactors for the enzymes are provided as a single lyophilized reagent that, when reconstituted, forms a proper reagent for use in the present amplification methods.

In some embodiments, the kit includes suitable buffers, reagents for isolating nucleic acid, and instructions for use. Kits can also include a microarray that contains nucleic acid or peptide probes for the detection of the mutant genes or encoded proteins, respectively.

In some embodiments, the kits can further contain a solid support for anchoring the nucleic acid or proteins of interest on the solid support. In some embodiments, the target nucleic acid can be anchored to the solid support directly or indirectly through a capture probe anchored to the solid support and capable of hybridizing to the nucleic acid of interest. Examples of such solid supports include, but are not limited to, beads, microparticles (for example, gold and other nanoparticles), microarray, microwells, multiwell plates. The solid surfaces can comprise a first member of a binding pair and the capture probe or the target nucleic acid can comprise a second member of the binding pair. Binding of the binding pair members will anchor the capture probe or the target nucleic acid to the solid surface. Examples of such binding pairs include but are not limited to biotin/streptavidin, hormone/receptor, ligand/receptor, antigen/antibody.

Exemplary packaging for the kit can include, for example, a container or support, in the form of, e.g., bag, box, tube, rack, and is optionally compartmentalized. The packaging can define an enclosed confinement for safety purposes during shipment and storage.

Provided herein is a kit for prognosing a vascular disease in a subject or patient diagnosed with a vascular disease (e.g., CAD), comprising at least one PCR primer pair for PCR amplification of a CCM pathway gene or at least one probe for hybridizing to a CCM pathway gene under stringent hybridization conditions. In some embodiments, provided herein is a kit for prognosing a vascular disease in a subject or patient diagnosed with a vascular disease (e.g., CAD), comprising: (i) at least one PCR primer pair for PCR amplification of a CCM pathway gene or at least one probe for hybridizing to a CCM pathway gene under stringent hybridization conditions; and (ii) at least one PCR primer pair for PCR amplification of at least one housekeeping gene.

In one aspect, the present disclosure provides a kit comprising one or more of the components described herein. In some embodiments, the kit comprises a vector system and instructions for using the kit. In some embodiments, the vector system comprises (a) a first expression regulatory element operably linked to a nucleotide sequence encoding an effector protein, or one or more nucleotide sequences encoding the effector protein; and (b) a second expression regulatory element operably linked to one or more nucleotide sequences encoding a single guide RNA (sgRNA) comprising a guide sequence capable of hybridizing to a target sequence. In some embodiments, components (a) and (b) are located on the same or different vectors. In some embodiments, the sgRNA is capable of hybridizing to one or more genes of the Cerebral Cavernous Malformation (CCM) pathway or one or more genes that regulate the CCM pathway (i.e., target genes). In some embodiments, the one or more genes are selected from the group consisting of: TLNRD1, CCM2, HEG1, ITGB1BP1, KRIT1, PDCD10, ARPC2, CDC42, CDH5, DNM2, MEAF6, PDCD7, RHOA, KLF2, KLF4, MAP2K5, MAP3K3, MEF2A, and NFAT5. In some embodiments, the host cell comprises components (a) and (b). In some embodiments, component (a), component (b), or components (a) and (b) are stably integrated into a genome of the host eukaryotic cell. In some embodiments, the effector protein is a CRISPR enzyme. In some embodiments, the CRISPR enzyme is codon-optimized for expression in a eukaryotic cell. In some embodiments, the CRISPR enzyme directs cleavage of one or two strands at the location of the target sequence. In some embodiments, the CRISPR enzyme lacks DNA strand cleavage activity. In some embodiments, the guide sequence is at least 15, 16, 17, 18, 19, 20, 25 nucleotides, or between 10-30, or between 15-25, or between 20-24, or between 18-24 nucleotides in length.

### EXAMPLES

These Examples are provided for illustrative purposes only and not to limit the scope of the claims provided herein.

### Example 1. Materials and Methods

The present example describes materials and methods used in the study described herein.

### Cell Culture & Creation of CRISPRi TeloHAEC

Telomerase-immortalized human aortic endothelial cells (TeloHAEC) were purchased from ATCC, and grown in Lifeline VEGF endothelial cell media (LL-0005) with 1× Penn/Strep. Cells were plated at a density of 0.5-1.0×10⁶ cells per 10 cm plate and split before reaching 4×10⁶/plate (3 to 4 days). To create the TeloHAEC CRISPRi line, cells were transduced with lentiviral vectors containing 1) dox-inducible (tetracycline operator controlled) dCas9-KRAB-BFP (CRISPRi machinery, which targets epigenetic repressors to efficiently silence enhancers or promoters, AddGene #85449) and 2) rtTA (tetracycline activator) with a hygromycin marker. After hygromycin selection (250 ug/ml for 4 days), cells were treated with 1 μg/ml doxycycline (dox, a stable tetracycline analogue) for 3 days before FACS sorting for the top 15% of BFP positive cells, and after a period in culture without dox, treated again with dox and re-sorted. Diagnostic FACS performed immediately before the Perturb-seq screen showed no leaky BFP expression in the absence of dox, and 93% BFP positive cells in the presence of dox. CRISPRi TeloHAEC were passaged for routine maintenance in the absence of dox. Eahy926 cells (a HUVEC+A549 hybrid line) were purchased from ATCC, and grown in DMEM+10% FBS. To study responses to CAD-associated cytokines, cells were untreated (control), or treated with 10 ng/ml recombinant human IL-1B (Millipore IL038), 10 ng/ml recombinant TNF-α (Millipore GF023) or with normal media lacking VEGF (for TeloHAEC) or supplemented with VEGF (1× concentration from LifeLine VEGF media, for Eahy926), for 24 hours.

### Bulk RNA-Seq

Total RNA was harvested from TeloHAEC (parental or CRISPRi lines) by Qiagen RNeasy kit (74016, Qiagen), DNAse treated (TURBO DNAse, In Vitrogen AM2238, 15′ 37° C.), and purified on MyOne Silane beads. For flow response and MAP3K3 knockdown studies, DNAse treatment was performed on the spin column between two buffer RW1 washes, for 20 mins at room temperature with Purelink DNAse (In Vitrogen 12185010), 10 μL in 80 μL of 1× buffer. mRNA was purified from 400 ng to 1 μg of total RNA using the NEBNext Poly(A) mRNA Magnetic Isolation module (NEB), and processed for RNA-seq library generation using the NEBNext Ultra II RNA Library Kit for Illumina (NEB), and sequenced to a depth of 10 to 30 million reads/library. Reads were mapped to the human hg19 genome build, and counts per gene tables assembled, or, for flow response and MAP3K3 knockdown studies, using Kallisto. Differential expression calls were made using Limma VOOM (for parental TeloHAEC & Eahy926) or edgeR (for all other libraries). Bulk RNA-seq data is available from the Gene Expression Omnibus (GEO). For cytokine treatment of parental lines and single guide knockdowns use accession GSE210522. For flow response and MAP3K3 double knockdown studies use accession GSE232400 (reviewer token: svcbwygehpklvqx).

### ATAC-Seq, H3K27Ac ChIP-Seq & Identification of TeloHAEC Enhancers

For ATAC-seq, one well of a 12-well plate (˜200,000 cells) was directly lysed using a custom TN5 buffer (33 mM Tris Acetate pH 7.8, 66 mM Potassium Acetate, 10 mM Magnesium Acetate, 16% dimethylformamide & 0.1% NP40). 47.5 μl of lysed cells was added to 2.5 μl Tn5 tagmentation enzyme (Illumina) & incubated at 37° C. for 1 hr, and the reaction stopped by addition of 20 μl buffer RLT (Qiagen). Products were purified by addition of 1.8 volumes Ampure XP beads (Beckman-Coulter) & magnetic separation of beads, followed by two 80% ethanol washes, brief drying of pellets & resuspension in 23 μl water. Barcoded ATAC-seq libraries were then generated, and sequenced to a depth of 10-20 million reads per library. Chromatin immunoprecipitation for histone H3 lysine 27 acetylation (H3K27ac) was performed. ChIP-seq libraries were prepared using the KAPA Hyper Prep Kit (KAPA Biosystems). ATAC-seq libraries were prepared in biological triplicate, and ChIP-seq libraries in biological duplicate. For both types of libraries, reads were mapped to the human genome (hg19 build) using Bowtie2, and ATAC peaks identified using MACS2. Raw and processed data can be found on GEO: GSE210489 (ATAC-seq) and GSE210491 (ChIP-seq). Enhancers and their predicted target genes were identified by applying the Activity-by-Contact (ABC) model to these data, using ATAC-seq and H3K27ac ChIP-seq as the measures of enhancer Activity, and using a cross-cell type average of Hi-C maps as the measure of 3D enhancer-promoter contact frequency (github.com/broadinstitute/ABC-Enhancer-Gene-Prediction). An ABC fractional score threshold of ≥0.015⁸ was used.

### Selection of Genes for the Perturb-Seq Library

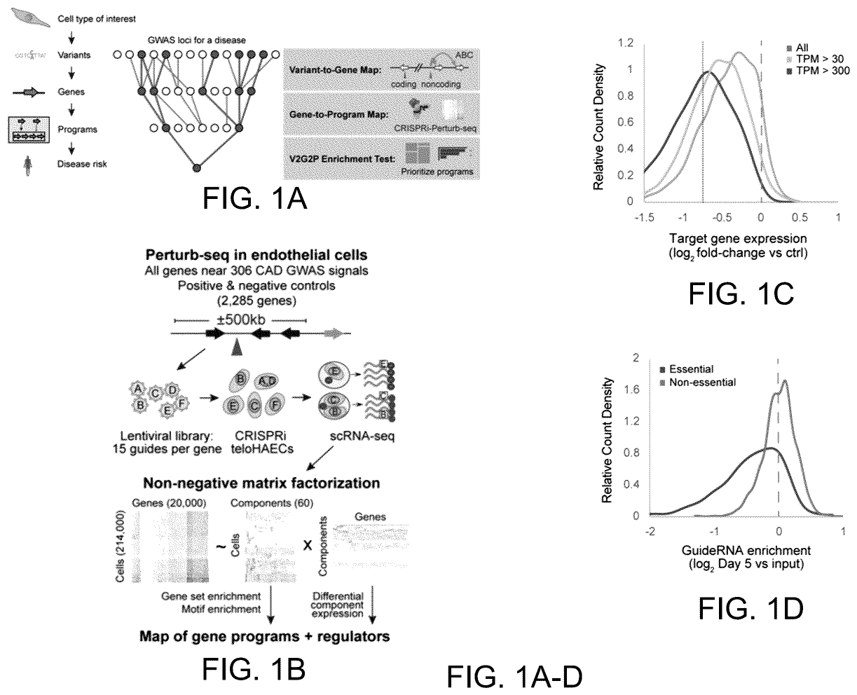

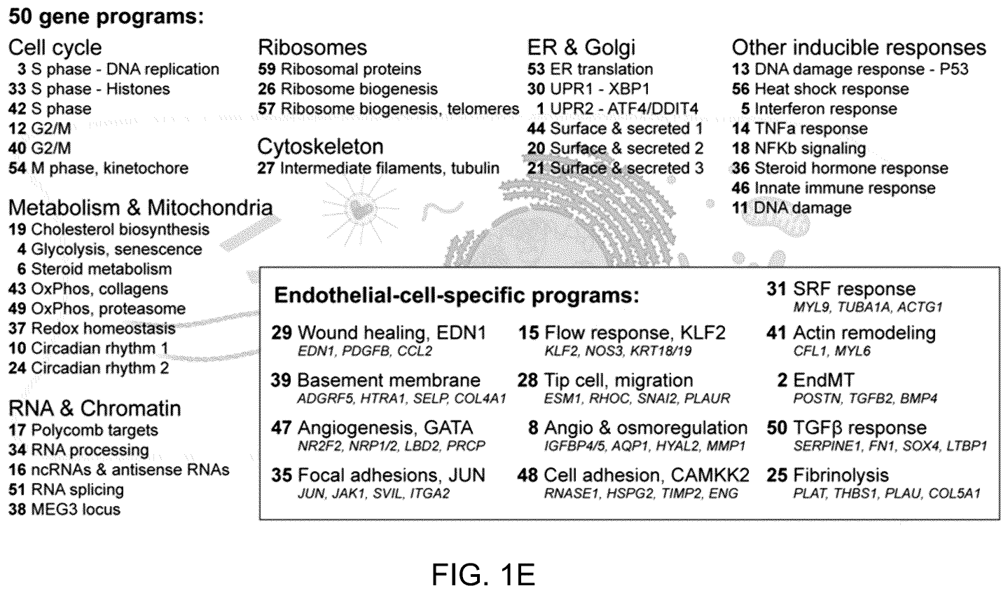

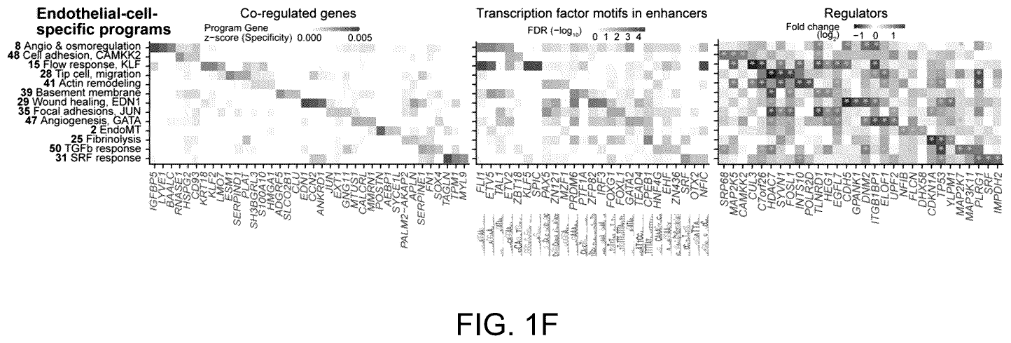

Perturb-seq, which involves knocking down hundreds to thousands of genes in parallel and measuring their effects on gene expression using single-cell RNA-seq, has previously been shown to provide a high-content, unbiased view of cellular programs as represented in gene expression. See, for example, Replogle, J. M. et al. Mapping information-rich genotype-phenotype landscapes with genome-scale Perturb-seq. Cell (2022) doi:10.1016/j.cell.2022.05.013. A library of promoter-targeted CRISPRi guides to all potential causal CAD genes was constructed (FIG. 1B). First, all coding genes within a 1 megabase window surrounding the lead SNPs from CAD loci identified in either or both of van der Harst et al (Harst, P. van der, van der Harst, P. & Verweij, N. Identification of 64 Novel Genetic Loci Provides an Expanded View on the Genetic Architecture of Coronary Artery Disease. Circulation Research vol. 122 433-443 Preprint at doi.org/10.1161/circresaha.117.312086 (2018)) and Aragam et al (Aragam, K. G. et al. Discovery and systematic characterization of risk variants and genes for coronary artery disease in over a million participants. bioRxiv (2021) doi:10.1101/2021.05.24.21257377) that were expressed in TeloHAEC (1+ TPM, from bulk RNA-seq) were identified. If fewer than 2 expressed genes were found within 500 kb up- or downstream of the lead SNP, the window was expanded to include the closest 2 genes to each side (for a total of 1661 genes). Non-coding genes were generally excluded, unless there was strong evidence for regulatory functions, particularly in ECs. In some cases, genes were included with TPM<1, particularly if they were known to be important for CAD in tissues where they were more highly expressed (e.g. PCSK9), or were regulated by IL1-beta in bulk RNA-seq data in TeloHAEC (FDR<0.05, fold change >1.3). As negative controls, guides targeting 48 coding genes expressed in other cell types but not detectably expressed in ECs were included, and the 132 expressed coding genes within 1 Mb of 16 randomly-selected lead SNPs associated with Inflammatory bowel disease, Crohn's disease or Ulcerative colitis, and which did not overlap with CAD loci. As positive controls, and to aid in connecting candidate CAD genes to known pathways in ECs, the promoters of an additional 284 genes with known roles in a wide range of CAD-relevant EC functions such as barrier formation, TGF-beta signaling and inflammation, as well as major classes of expressed transcription factors and common essential genes, were targeted. An additional 160 promoters of expressed genes predicted to be regulated by EC enhancers containing fine-mapped variants associated with other disease phenotypes expected to be modulated by ECs (migraine, blood clotting in leg, systolic blood pressure, diastolic blood pressure & mean arterial blood pressure, from UKBB) were also targeted. This gave a total of 2285 genes, some of which were members of more than one category.

### Guide Library Production and Validation

sgRNA guides were designed to target promoters of the chosen CAD and control genes (15 guides spanning from −150 to +100 relative to the Transcription Start Site (TSS)), using an established pipeline (github.com/EngreitzLab/CRISPRDesigner). To optimize expression, if the first base of a guide was not G, a G was added to the 5′ end. 400 non-targeting guides (that don't match any region of the genome) and 600 safe targeting guides (targeting non-genic regions lacking enhancer marks) were included. Because TeloHAEC are puromycin resistant, the CROP-opti vector (Addgene, #106280) was adapted for Blasticidin resistance (“Crop Opti Blast”), by digesting the vector with BsiWI and MluI, PCR-amplifying the Blasticidin resistance gene from lenti-dCas-VP64_Blast (Addgene, #61425) with added homology arms, and performing Gibson Assembly (Gibson Master mix, New England Biolabs). To create “CROP-opti-BC-Blast”, HyPR-Seq barcodes were added between the WPRE element and the U6 promoter of CROP-opti-Blast. A pool of oligos encoding the guide sequences, plus extensions with homology to the u6 promoter and downstream scaffold (TATCTTGTGGAAAGGACGAAACACCG (SEQ ID NO: 230) & GTTTAAGAGCTATGCTGGAAACAGCATAG (SEQ ID NO: 231)) was synthesized by Agilent Technologies, and cloned into Crop Opti BC Blast by Gibson assembly and bacterial electroporation, with an average of 202 transformants per guide. Note that, since the vector was prepared from a single clone, diversity of the HyPR-seq barcodes (which were not required for Perturb-seq) was not preserved. The library was sequenced and shown to include all 37,637 designed guides with relatively equal coverage of each (the difference in count frequency between the top and bottom 10th percentiles of guides was 2.8). A lentiviral library was produced using a standard 3 plasmid protocol, at a scale to yield 10 ml of virus, stored in aliquots at −80° C., with each aliquot thawed only once.

### Perturb-seq: Experimental Procedure

To transduce this library into CRISPRi TeloHAEC, cells were resuspended in media containing 10 μg/ml polybrene at a density of 1e6 cells per ml, mixed with virus and plated 4 ml per well to 6-well plates, centrifuged at 2000 rpm for 2 hrs at 30° C., and incubated at 37° C. for 2 hours before addition of another 4 ml media without polybrene. The next day, cells were harvested and plated to 15 cm plates and treated with 15 μg/ml blasticidin for 4 days. The effective viral titer was determined using this same high yield protocol, and a volume of virus was chosen that gave a final measured 15.7% infection rate (such that most successfully transduced cells have only 1 guideRNA). For the Perturb-seq study, 127.5 million CRISPRi TeloHAEC were transduced and selected for blasticidin resistance, for a coverage of approximately 360 cells per guide (as back-calculated from yield at the first post-blasticidin split, using the 36.7 hour doubling time observed in routine culture) to 461 cells per guide (as estimated from initial number of cells and infection rate). After blasticidin selection, cells were treated with 2 μg/ml dox for 5 days (plating 18e6 cells at each split, to maintain complexity of the library). Since atherosclerotic plaques develop slowly, the longer-term transcriptional effects of causal CAD gene disruption would provide the greatest insights into disease mechanisms. Thus, while it was found that knock down of guide-targeted genes is near maximal after 2 days of doxycycline treatment (inducing the CRISPRi machinery), guide-containing cells were treated with 2 μg/ml doxycycline for 5 days, to measure the long term consequences of each perturbation. The same 5-day dox treatment protocol was used for downstream validation studies (e.g. bulk RNAseq of single gRNA clones).

Unlike other scRNAseq methods, CROPseq allows multiplets (droplets containing 2 or more cells) to be unambiguously identified (as droplets containing more than one guide). This allowed us to load ˜10-fold more cells per 10× Genomics lane than the maximum number recommended in the manufacturer's protocol. Briefly, cells were harvested, resuspended in PBS with 1% BSA, counted, and loaded at 150,000 cells per lane on a 10× Genomics Chromium Controller using a 3′ scRNA-seq V3 kit (20 lanes, for a total of 3 million cells). Cells were isolated in two batches, with 6 lanes for the first batch, and 14 lanes, across 2 cassettes, for the 2nd batch, 6 hours later. scRNA-seq libraries were generated using the 10× protocol, and given lane-specific indexes. From the initial amplified cDNA, a two stage PCR protocol was used to generate “dialout” libraries, for each lane. Because the CROP-seq vector expresses a Pol II polyadenylated transcript that ends just downstream of the guide sequence, the dialout libraries identify the sgRNA sequences associated with each droplet. PCR1 oligos were: CTACACGACGCTCTTCCGATCT (SEQ ID NO: 232) & GTGACTGGAGTTCAGACGTGTGCTCTTCCGATCTTGTGGAAAGGACGAAACACC (SEQ ID NO: 233), and PCR2 oligos were AATGATACGGCGACCACCGAGATCTACACTCTTTCCCTACACGACGCTC (SEQ ID NO: 234) & CAAGCAGAAGACGGCATACGAGAT-8 bp index sequence-GTGACTGGAGTTCAG (SEQ ID NO: 235).

Assignment of gRNAs to Cells

To get complete information about guide assignments, dialout libraries were sequenced to approximately 40-fold saturation. Guides were identified from read 1 sequences, using Bowtie2 to align dialout reads to a “genome” composed of all 37,637 guide sequences, requiring no-mismatches. Aligning read 1 and read 2 sequences linked gRNA sequences with cell barcodes (CBCs, unique to each bead/droplet) and unique molecular identifiers (UMIs). To avoid low-frequency PCR chimeras, each CBC-UMI-guide combination was required to be duplicated at least 4-times. Then the guides associated with each CBC were identified, and the number of different UMIs for each CBC-guide combination. 4 UMIs were selected for any single guide as the threshold to call a cell as containing a guide. Singlets (one cell & one guide per CBC) were defined as having >=4 UMIs for the most frequent guide and >=4× less than this for the 2nd most frequent guide (choosing these thresholds to give a good balance between power to detect transcriptional effects and accuracy in measuring the magnitude of these effects, as described under Selection of Singlet Thresholds, below). Doublets and higher multimers, were cells with >=4 UMIs for the top guide, and one or more additional guides with more than ¼ this number of UMIs.

scRNA-Seq Data Pre-Processing and Subsetting to Singlets

scRNA-seq libraries were sequenced on two Illumina NovaSeq S4 flowcells, yielding 20,245,734,673 total reads, across all 20 libraries. The FASTQ files were processed on the 10× Cloud to run cellranger count with the hg38 reference genome. The “filtered” features (i.e., cell barcodes corresponding to droplets that contain a cell) were used, and combined the outputs from all twenty 10× lanes into a single genes×cell matrix. This analysis identified 822,156 cell-containing droplets. To measure the effects of individual guides on individual cells, only those CBCs identified in the dialout analysis as corresponding to singlet cells were selected. This identified 214,449 singlets (droplets containing one cell and one guide), defined as 4+ unique molecular identifiers (UMIs) for the top guide and <=4-fold fewer UMIs for any other guide. This gave an average of 5.7 cells per guide and 85.5 cells per target promoter. Average sequencing depth was 10,870 transcriptome-mapped UMIs per singlet cell, and 929,000 transcript UMIs, across all 15 guides, for each target promoter. Raw and processed data, as well as supplemental files for downstream analyses, can be found on GEO: GSE210681.

### Estimation of Fitness Effects

To estimate the fitness effects of guides, the relative frequency of all 15 guides was compared to a given target in the original library to the frequency of the same guides in singlet cells, and estimated significance by Benjamini-Hochberg adjusted binomial tests. Essential genes were defined as those that scored as fitness reducing in 5 of 7 tested lines.

### Differential Gene Expression (DE) Analysis & Knockdown Efficacy

To measure the differential effects of guides to specific target promoters on the transcriptome, edgeR was used, comparing all singlet cells with guides to each target to all singlet cells with any of the 1,000 non-targeting and safe targeting guides. Genes with fewer than 10 UMI counts across all singlet cells were excluded from the analysis. To control for possible batch effects, the 10× lane number was included as a covariate. For average knockdown efficacy (across all 15 guides), the log 2 fold change and p-values reported by edgeR for each target gene in cells with guides to that target were used. To measure the knockdown efficacy of individual guides, binomial tests were used on the number of transcripts for the target in singlet cells with the corresponding guides (hits), all transcripts in singlets with guides to the target (tests) versus a background frequency of transcripts to the target over all transcripts in all other singlet cells. Note, that with an average of 5.7 cells per guide, assigning significance for knockdown effects of individual guides was only possible for genes with high expression in unperturbed cells (e.g. TPM>100). To identify perturbations with a significant effect on the transcriptome, the edgeR results for the 48 negative control promoters (for genes not detectably expressed in TeloHAEC) were used to estimate the number of apparently DE genes that occur by chance, at thresholds of nominal p.value 0.005 and fold change of 1.15. Perturbations with a significant effect on the transcriptome (across all 15 guides to each target) were identified as having more DE genes, by these same thresholds, than the 48 non-expressed controls (using binomial tests with a background rate equal to the average DE gene count for controls over all genes tested, and multiple hypothesis correction by the Benjamini Hochberg method).

### Selection of Singlet Thresholds

Expression of the CROP-seq guide mRNA in TeloHAEC is lower than in some other cell lines, such as K562 & HEK293T resulting in the absence of a clear gap between noise (low UMI CBC-guide combinations that are likely PCR chimeras) and higher UMI-count true guide reads. It was hypothesized that reducing stringency for singlet calls could potentially reduce power to detect perturbation effects on transcription (due to increased noise from mis-calling some true doublets as singlets), or could increase power (by increasing the total number of called singlets analyzed). To test which of these was true, the correlation between differential expression calls for cells with guides to a given target in the full Perturb-seq library versus a smaller pilot library tested in resting TeloHAEC was measured, reasoning that parameters that improved the correlation between these separate studies would also increase the power of the full scale library to detect real transcriptional effects. Information about guides, as well as raw and processed data for the “200 gene” pilot library can be found on GEO, with accession number GSE212396. For the pilot library, singlets with the very stringent threshold of 6 UMIs for the top guide and more than 5-fold less for the next most frequent guide (“6&<5×”) were chosen. For the full Perturb-seq dataset, 4 UMIs for the top guide and equal to or more than 4-fold less than the next most frequent guide (“4&<=4×”, final applied standard, yielding 214,449 singlets), or the relaxed thresholds “3&<=3×” (284,466 singlets) and “2&<=2×” (389,792 singlets) were chosen. 37 gene targets were identified that were shared between libraries, and which also showed an FDR<0.1 effect on the transcriptome in the full Perturb-seq 4&<=4× dataset (measured as described above). EdgeR was then run for differential expression testing (cells with guides to each of these 37 targets versus cells with control guides), for each library and singlet definition (pilot 6&<5×, or full library 4&<=4×, 3&<=3×, and 2&<=2×). Then, for all genes called as differentially expressed in either the pilot library or the full library (raw p-value <0.01), the correlation in log 2 fold changes between the pilot & full scale data was measured, repeating this analysis for each singlet definition.

Lastly, the difference in correlation coefficients (R) between the relaxed threshold comparisons (pilot v. full library 3&<=3×, and pilot v. full library 2&<=2×) and the base comparison (pilot vs. full library 4&<=4×) were measured. It was found that the median correlation between pilot & full-scale studies significantly improved with the relaxed singlet thresholds (with significance assessed by two-sided t-test). This indicates that the increased number of called singlets with the relaxed thresholds increased the power to detect real transcriptional effects, despite an expected increase in doublets mis-assigned as singlets. Plotting change in R for each target for the 2&<=2× singlet definition ((R for pilot v. full library 2&<=2×)-(R for pilot v. full library 4&<=4×), y-axis) against the R value for the base correlation (between the pilot and the 4&<=4× full library singlet definition, x-axis), it was found that in all 13 cases where R started high (>0.15, likely real correlations between strong transcriptional effects), R increased. R also increased in all but one case where it started out negative (correcting anti-correlations likely driven by noise). Weak positive base correlations were adjusted up or down, potentially improving true correlations and correcting spurious ones. As such, relaxed singlet thresholds might improve power to detect reproducible transcriptional changes more than is indicated by simple mean differences in R values. On the other hand, it was found that lower stringencies reduced the apparent knock down effect on these target genes, themselves (median log 2 fold changes: −0.53 for 4&<=4×, −0.41 for 3&<=3× and −0.42 for 2&<=2×), likely due to the fact that a mis-called singlet that was actually 2 cells with different guides would show half-magnitude transcriptional effects of each guide. Reduced singlet thresholds also decreased median log 2-fold changes for target genes across all targets in the full-scale library (−0.368 for the 4&<=4× singlet definition, and −0.327 for the 2&<=2× singlet definition). Based on these observations, the thresholds of 4 UMIs for the top guide and <=¼ this for the next (4&<=4×) were chosen, to provide a good balance between overall power and accurate detection of the magnitude of effects.

### Data Processing Prior to Defining Gene Programs

To remove noncoding RNA from the analysis, genes with names starting with “LINC” and gene names with patterns starting with two alphabets and six digits were removed. Cells with a minimum of 200 unique detected genes and a minimum of 200 UMIs were retained. Genes detected in a minimum of 10 cells were retained.

Consensus Non-Negative Matrix Factorization (cNMF)

To identify sets of genes that are co-expressed across single cells in a dataset, non-negative matrix factorization (NMF) was used. NMF decomposes an input cell×gene read count matrix (X) into a cell×component matrix (W) and a component×gene matrix (H), such that X=W·H+E, where E is the error term. The cell×component matrix W represents the contribution of each component to the cell's transcriptional profile, and the component×gene matrix H encodes information about gene expression programs. The number of components (K) is a hyperparameter defined prior to performing matrix factorization (see below). To account for the fact that the NMF algorithm is a stochastic algorithm that depends on the initial seed, the consensus NMF (cNMF) method was used. The cNMF method, after normalizing each gene's expression to unit standard deviation, factorizes the normalized matrix multiple times (here, 100 repeats); clusters the components from the repeat runs based on their pairwise Euclidean distances; removes the components that show low similarity to any other component (here, threshold on Euclidean distance=0.2); defines “consensus components” as the median of each of the component clusters; and recomputes the cell×component matrix W using these consensus components. As one technical note about applying the cNMF pipeline, it was found that including all genes, as opposed to the 2000 most variable genes, was important for finding certain programs observed only infrequently in the dataset. This is because genes whose expression changes in only a small fraction of cells (e.g. cells with a particular perturbation) would not end up being included in the 2000 most variable genes.

Choosing the Number of Components for cNMF Analysis

To choose the free parameter K (number of components), a set of benchmarking statistics was defined, and the results of cNMF run for K=[3, 4, 5, 6, 7, 8, 9, 10, 11, 12, 13, 14, 15, 17, 19, 21, 23, 25, 27, 29, 30, 35, 40, 45, 50, 55, 60, 100] were compared. K=60 was ultimately chosen for all downstream analyses.

The following benchmarking statistics were examined:
(i) Number of unique GO terms enriched in program co-regulated genes;
(ii) Number of unique enriched TF motifs in the promoters or enhancers of program co-regulated genes;
(iii) Number of perturbations significantly regulating any component;
(iv) Error of cNMF (difference between the original normalized data and reconstructed data, calculated by taking the sum of squares of the element-wise difference of the data.); and
(v) Stability of cNMF (a measure of consistency of the components output from repeated runs, represented by the silhouette score).
K=60 was chosen for further analysis, as the number of components that gave a low cNMF error value while near-maximizing each other metric.
   Excluding Components Associated with Batch Effects

It was examined whether some components identified by cNMF were likely to represent batch effects. To do so, the Pearson correlation between each of the 20 batches (i.e., 10× lanes) and the expression of each component across all cells was calculated. Based on the distribution of batch×program Pearson correlation, 10 components with Pearson correlation >0.15 were assigned as likely representing batch effects. The remaining 50 components were used for further analysis. This approach (including batch as a covariate in the differential expression test) has theoretical advantages, in particular reducing bias when groups (here, perturbed genes) are not distributed evenly across batches.

### Defining Co-Regulated Genes for Each Program

‘Co-regulated genes’ were defined for each cNMF component as the 300 marker genes with the highest z-score regression coefficient as defined by cNMF. Essentially, cNMF uses a linear regression model to identify coefficients indicating the number of standard deviations each gene's expression would change with the increased usage of a given component. A component's marker genes, then, are those with the highest “marker gene regression coefficients” (or “specificity scores”) for that component, and the top 300 of these marker genes were selected as the set of “co-regulated genes” for each gene expression “program” (as defined below).

### Defining Regulators for Each Program

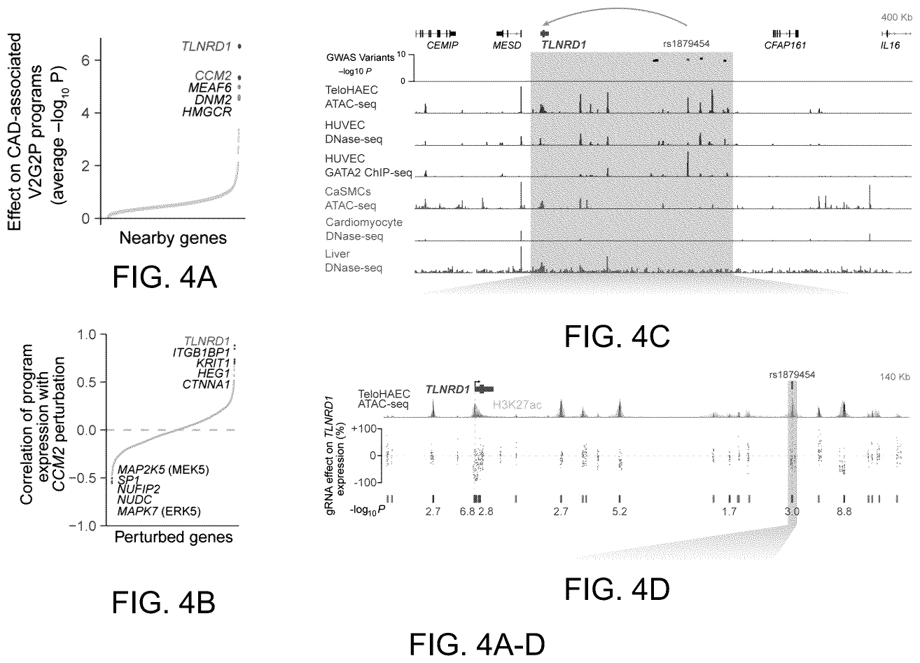

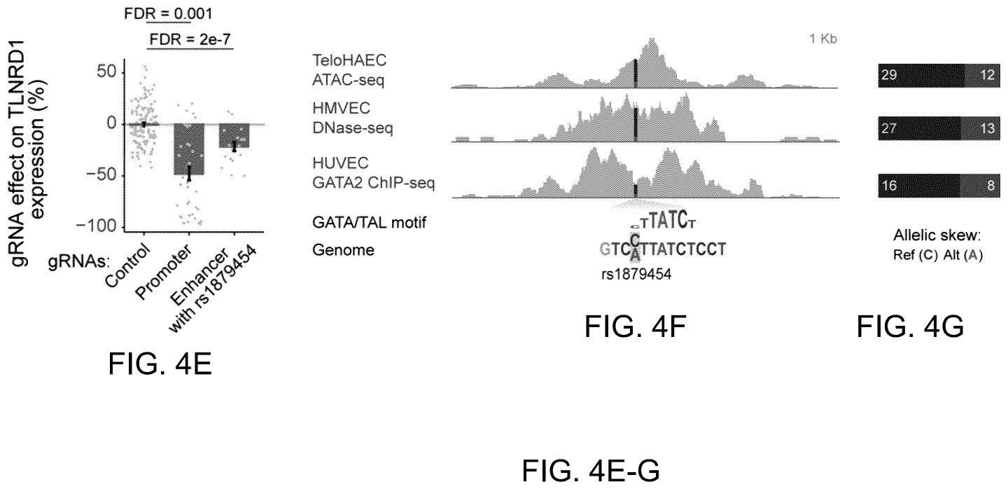

It was tested whether gRNAs targeting a given gene led to a significant change in expression of each component from the cNMF model. The Model-based Analysis of Single Cell Transcriptomics package (MAST) was used to compare the expression of each component in cells carrying gRNAs targeting a given gene vs. cells carrying control gRNAs (1,000 safe-targeting and negative control guides), including 10× lane as a covariate to account for batch effects. The guides present in fewer than 3 singlet cells and the perturbations with fewer than 2 guides were removed. The Benjamini-Hochberg method was used to account for multiple hypothesis testing on the MAST p-values (Extended Data FIG. 4e), and assigned ‘regulators’ of a program as those genes whose perturbation affected component expression with FDR<0.05 accounting for 140,760 total tests (60 programs×2,346 perturbations, which includes the 2,285 targeted TSSes, as well as targeted enhancers that were not further analyzed in this study.

To confirm that these FDRs were well-calibrated, a simulation-based test was also conducted. For each perturbed gene, from the control cells (all singlet cells with non-targeting or safe-targeting guides) the same number of cells were sampled, and compared these sampled cells to the rest of the control cells using the same MAST procedure. 0 significant regulators were identified in this approach, indicating that the FDR<0.05 threshold is a conservative estimate. The same procedure was performed to estimate the background rate for perturbations called as having a significant effect on the transcriptome using EdgeR.

### Definition and Annotation of Gene Expression Programs

A gene expression program was defined as the set of genes comprised of both the 300 “co-regulated genes” and the significant “regulators” for each cNMF component. Programs were annotated based on features of their co-regulated genes and regulators, including: by manual curation of genes with known biological functions, by enrichment of transcription factor (TF) motifs in the promoters and predicted enhancers of co-regulated genes, and by GO term enrichment (see below).

### Identifying Motifs Enriched in Promoters and Enhancers

To identify transcription factors that might regulate program co-regulated genes, enrichment of human transcription factor motifs in the sequences of the promoter and enhancers of the top 300 genes ranked by component specificity score was calculated.

Promoter sequences were obtained by taking 500 bp surrounding the TSS as previously annotated and enhancer regions from the Activity by Contact model at an ABC score threshold of 0.015 in the TeloHAEC control condition. For a gene that had multiple enhancers, motif instances were counted across all of its enhancers. To match motifs to sequences, HOCOMOCO v11 human full scan motifs (hocomoco11.autosome.ru/downloads_v11), and Find Individual Motif Occurrences (FIMO) (meme-suite.org/meme/meme_5.3.2/tools/fimo) were used, with the default settings and p-value threshold of 10⁻⁶ for enhancers, 10⁻⁴ for promoters.

For a given motif and a given program, the number of occurrences of a motif in the promoter sequences of either (i) the top 300 program co-regulated genes, or (ii) all expressed genes in TeloHAEC was counted, and these two vectors of motif counts were compared using a t-test. Enrichment was computed by dividing the program gene's average motif match count by the rest of the expressed gene's average motif match count. All pairs of matched motifs (570 for promoter and 590 for enhancer)×60 programs were tested, and Benjamini-Hochberg method was used to account for multiple hypothesis testing on the t-test p-values.

### Determining Enrichment of Annotated Gene Sets in Components

To determine if the gene expression programs align with annotated and publicly available pathways, whether the co-regulated genes in each component were enriched in gene sets from the Molecular Signatures Database (MSigDB) was tested. To do so, clusterProfiler R package and MSigDB gene sets were used (here, the gene sets labeled as “all” for all gene sets and “c5” for GO terms only). The MSigDB gene sets were filtered such that they consist of greater than 3 genes and less than 800 genes. Each program was annotated with the gene sets that showed significant enrichment among the program genes (FDR<0.05), and the number of gene sets showing significant enrichment as a function of the number of programs K was compared.

### Defining Endothelial-Cell-Specific Programs

To annotate programs as “endothelial-cell-specific” or “EC-specific”, the degree to which program genes were expressed in endothelial cells versus other cells was analyzed. Gene expression transcript per million (TPM) data across all available cell types from FANTOM5 was taken, and the expression z-score of each gene across all cell types was calculated. To give each gene an EC-specificity score, the average of all z-scores for a gene across endothelial cell samples was calculated. A high EC-specificity score means the gene is specifically expressed in ECs. EC specificity scores were defined for each program as the average of the 300 program genes' EC specificity score, and selected 0.19 or 90% percentile for threshold to call EC-specific programs.

Variance Explained by all cNMF Components

To quantify the fraction of variance explained by all 60 programs jointly, the residual variance in the dataset after subtracting the consensus matrix factorization was compared to the total variance in the dataset:

$$V = 1 - \frac{Var ( X - WH )}{Var ( X )} ,$$

where X is the (cell×gene) normalized data matrix input to cNMF, W is the (cell×program) usage matrix, H is the (program×gene) spectra or weight matrix, and matrix variance is defined by summing the column- or gene-level variances:

$$Var ( X ) = {\sum }_{j} Var ( {X}_{j} ) .$$

Note that cNMF normalizes the input data so each Var(Xⱼ)=1.

### Variance Explained by Individual Gene Programs

To rank gene programs by variance explained, a method was devised to quantify variance explained by NMF or cNMF components separately. For the k'th program Hₖ, the effective matrix decomposition given only this program was considered; the effective usage matrix Bₖ in this case is given simply by orthogonal projection or ordinary least squares: Bₖ=XH′ₖ/∥Hₖ∥², where the prime indicates transposition. The variance explained in terms of the residual fraction was then defined as above:

$${V}_{k} = 1 - \frac{Var ( X - {B}_{k} {H}_{k} )}{Var ( X )} .$$

The method may be generalized to any set of programs, but with more than one program the effective usage matrix must be obtained by nonnegative least squares (a single iteration of NMF).

### Defining Variants in CAD GWAS Signals for Variant-to-Gene Analysis

CAD lead GWAS SNPs were derived from both Aragam et al. and Harst et al. (as discussed supra). Lead SNPs from Harst et al. were excluded if the SNPs were in strong LD (r²≥0.7) with an Aragam et al. lead SNP or were <=5 Kb away from an Aragam et al. lead SNP. An LD-expansion was performed to include SNPs that are both within a 1 Mb window of, and are in strong LD (r²≥0.9) with the any of the lead GWAS SNPs in 1000 Genome European ancestry (plink --ld-window-kb 1000 --ld-window 99999 --ld-window-r2 0.9). For each lead SNP, variants prioritized through functionally informed fine-mapping (PIP≥0.1) in either study were included. “GWAS Signal” was defined as this collection of variants around, and including, each lead variant.

Identifying CAD Variants Associated with Lipid Levels

CAD GWAS signals were classified as “lipid” or “non-lipid” based on their association with lipid levels in other GWAS studies, because the CAD GWAS signals also associated with lipids are presumed to act through non-endothelial cells such as hepatocytes. For lead signals included in Aragam et al., a CAD GWAS signal was considered to be associated with lipid metabolism if the lead variant is linked to “LDL-direct”, “Triglycerides”, “Cholesterol”, “HDL-cholesterol”. “Apolipoprotein A”, “Apoplipoprotein B”, “HDLC” or “LDLC” in the phenome-wide association scan (PheWas) conducted by Aragam et al. For GWAS signals exclusively nominated by Harst et al., a signal was linked to lipid metabolism if it reaches genome-wide significance (WGS) P-value threshold of 5×10⁻⁸ in relevant UK biobank GWAS studies (HDLC, LDLC, TG, ApoA, or ApoB) (Hilary Finucane and Jacob Ulirsch: www.finucanelab.org/data). The remaining GWAS signals not associated with lipid levels were referred to as “non-lipid CAD GWAS signals”, and this subset of signals was focused on as cases where CAD variants might plausibly act in endothelial cells.

### Linking Variants to Genes

A combination of variant-to-gene methods was used to identify a list of genes linked to CAD variants that could plausibly act in endothelial cells. At each CAD GWAS signal, at least two genes upstream or downstream of the lead GWAS SNP were considered as candidate genes, and all the genes within +/−500 Kb of the lead variant were considered to be potentially regulated by the locus. The analysis was focused on protein-coding genes and excluded long noncoding RNAs (“\hat{ }LINC”), gene isoforms (“−AS”), microRNAs (“\hat{ }MIR”), small nuclear RNAs (“RNU”), and genes of uncertain functions (“\hat{ }LOC”). To link CAD variants to genes, the CAD variants were intersected with endothelial cell-specific ABC enhancers in endothelial cells to identify the top two genes most likely to be regulated by each variant (highest 2 ABC fractional scores over 0.015). Specifically, ABC data was used, for enhancers and predicted target genes, from TeloHAEC and Eahy926 (control, or treated with IL1β, TNFα or VEGF, this study), and from prior ABC analysis of HUVEC. To account for cell state-specific regulation that was not predicted by ABC, CAD variants at each locus were also intersected with ATAC peaks identified in endothelial cells to identify the closest variants containing ATAC peaks for each potential target gene of the locus, and the top 2 genes closest to a CAD variant-containing ATAC peak are considered to be genes as plausibly regulated. Variants were also linked to genes if the variant was in a coding sequence or within 10 bp of a splice site annotated in the RefGene database (downloaded from UCSC Genome Browser on 24 Jun. 2017). It was confirmed that these CAD variants were significantly enriched for matching any or all of these criteria. 254 candidate CAD genes were identified, defined as “genes with V2G (variant-to-gene) links”, at 125 of 228 non-lipid CAD GWAS signals.

Transcription Profile Comparisons Between teloHAEC and Human Right Coronary Artery Endothelial Cell (RCAEC)

To confirm the validity of teloHAEC as a relevant model for endothelial cells in human coronary artery (where atherosclerosis that leads to CAD develops), single cell RNA-seq gene expression from control guide carrying teloHAEC from the Perturb-seq screen was compared to scRNAseq data from explanted human right coronary artery endothelial cells (RCAECs). The gene expression was compared at two levels: for all perturbed genes (2,285 genes) and for the 41 CAD associated genes. Among the perturbed genes in teloHAEC, 2,107 genes are expressed at TPM>1 in healthy or disease RCAECs. High correlation of gene expression in transcripts per million (TPM) between teloHAECs and RCAECs (Pearson correlation=0.66, p-value=6.45×10⁻²⁸⁰) was observed. Similar correlations of gene expression for the 41 CAD associated genes (Pearson correlation=0.63, p-value=9.29×10⁻⁶) was observed. Furthermore, 40 out of 41 CAD associated genes are expressed at >1 TPM in RCAECs.

### Identifying CAD-Associated Programs Via Variant-to-Gene-to-Program Analysis

An approach was developed to identify gene programs likely to affect CAD risk through functions in endothelial cells. To do so, it was tested whether the 254 genes with V2G links (between CAD variants and enhancers/coding regions in endothelial cells) were enriched in each Perturb-seq program. Specifically, a one-tailed Fisher exact test was performed separately for co-regulated genes and for regulators. For co-regulated genes, a contingency table was constructed for whether a gene is a co-regulated gene (out of 17,472 expressed genes) and whether a gene has a V2G link. For regulators, a contingency table was constructed for whether a gene is a regulator (out of all perturbed genes) and whether a gene has a V2G link. The p-values from co-regulated gene and regulator Fisher exact tests were then multiplied together to get a final program enrichment p-value. The Benjamini-Hochberg method was used for multiple hypothesis correction across all 50 non-batch programs. 5 programs showed significant enrichment by this method (FDR<0.05: Programs 8, 35, 39, 47, 48), referred to as “CAD-associated programs”.

### Defining CAD-Associated V2G2P Genes

“V2G2P genes for CAD” were defined as those 41 genes that were both (i) a gene with a V2G link to a CAD variant and (ii) a member of one of the 5 CAD-associated programs (as a regulator and/or co-expressed gene). The 41 genes were linked to 43 GWAS signals due to cases where independent GWAS signals are linked to the same gene.

### Identifying Enriched Programs Via MAGMA

To identify programs that were enriched for CAD heritability, the lists of co-regulated genes for each program (the top 300 genes most specifically associated with each cNMF component) were taken, and whether these genes were enriched in variants associated with CAD using MAGMA was tested. To do so, the CAD summary statistics from Aragam et al. (CAD_UKBB.gz) was taken, and the MAGMA --annotate function was used to summarize CAD association p-values for variants within a 50 kb window of all human genes, using the 1000 genomes European reference data to for base allelic frequencies (ctg.cncr.nl/software/MAGMA/ref_data/g1000_eur.zip). MAGMA was then run to test for enrichment of CAD heritability within 50 kb of the top 300 program genes, and corrected for multiple testing (60 programs) using the Benjamini-Hochberg method.

### Identifying Programs and Cell Types Enriched for CAD Heritability Via Stratified LD Score Regression

S-LDSC was used to estimate the enrichment of CAD heritability linked to program genes and to enhancers in TeloHAEC. To estimate the enrichment of CAD heritability linked to program genes, while the original implementations of S-LDSC linked variants to genes based on genomic distance, it was additionally required that variants either overlap exonic regions of the gene or overlap nearby candidate enhancers in endothelial cells. In particular, for the genes expressed in each program, an annotation was derived for S-LDSC by including exonic regions (exons from transcripts with Ensembl_canonical, appris_principal, appris_candidate, or appris_candidate_longest tags, as indicated in the GENCODE v38lift37 annotations) as well as endothelial cis-regulatory elements derived from snATAC-seq, from which the 9 adult and 8 fetal sets of endothelial peaks were merged into a single annotation, and for each geneset included all peaks within 50 kb of the gene starts and ends. For all peaks, coordinates were converted from the GRCh38 to the GRCh37 reference assembly using UCSC LiftOver, discarding peaks that could not be converted. To estimate the enrichment of CAD heritability in TeloHAEC enhancers, it was required that the variants overlap enhancers predicted by ABC from ATAC-seq and H3K27ac ChIP-seq data in TeloHAEC under control conditions or treated with IL1b, TNFa or VEGF (ABC score >0.015). LDSC was run, using 1000G EUR Phase3 genotype data to estimate LD scores, baseline v2.2 annotations as recommended by the LDSC developers, and HapMap 3 SNPs excluding the MHC region as regression SNPs. Gene sets or enhancer sets were then ranked by their enrichments and reported the p-values of these enrichments.

### Polygenic Priority Score (PoPS)

POPS is a method to nominate likely causal genes in a GWAS locus, which prioritizes genes based on their being members of many gene sets enriched for heritability genome-wide. PoPS was applied to summary statistics from Aragam et al. using the predefined set of gene sets as previously described. For each GWAS signal, the POPS rank among “nearby genes” (2 to either side of the lead SNP, and all within +/−500 kb) was calculated. It was previously shown that genes with the highest POP score in the locus are strongly enriched for likely causal genes, as identified by fine-mapped coding variants, and that this enrichment increases when further focusing on genes that are both the closest gene and have the highest PoP score. In this analysis, no features from Perturb-seq were used, and as such this method represents an entirely independent method that validates the high likelihood of causality of the set of CAD-associated genes.

### Defining Gene Expression Programs for Cells Carrying Control Guides

To examine the gene programs in normal teloHAECs, the same analysis pipeline was used on the subset of cells carrying control guides (5,506 cells). cNMF was used to discover K=60 components, and defined 60 “control programs” based solely on the 300 co-regulated genes defining each component (because control guides did not target any genes, so there was no regulator information). Of the 60 programs, 4 programs correlated with batch (Programs 2, 17, 22, 41). The program co-regulated genes between control cells and full library programs was compared. Control program 10 highly overlapped with full library programs 8 and 39. The four control programs that correlated with batch also had high overlap in co-regulated genes with the full library's batch programs. The V2G2P approach was utilized to prioritize these programs, and it was found that none of the control programs was enriched for genes with V2G links.

### Identifying Genes in the CCM Pathway

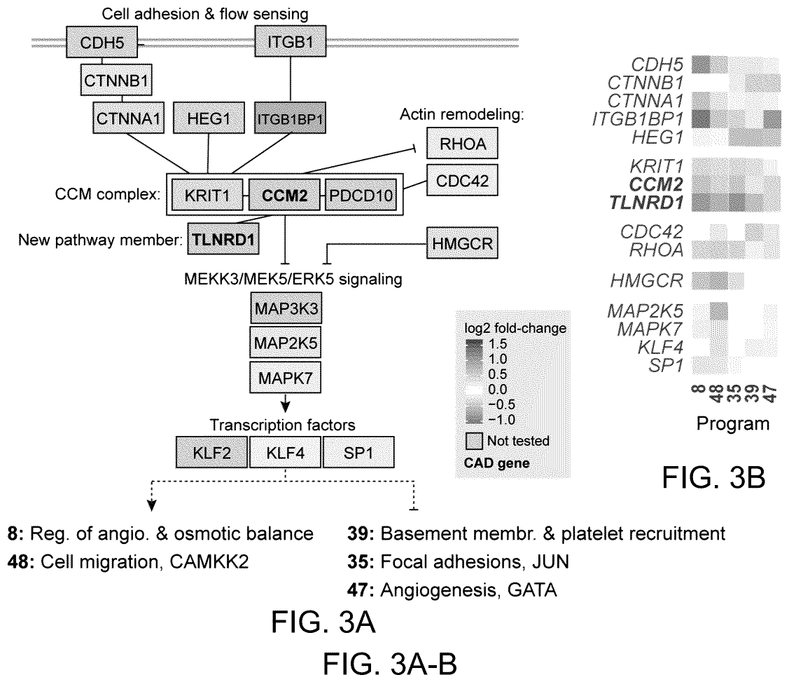

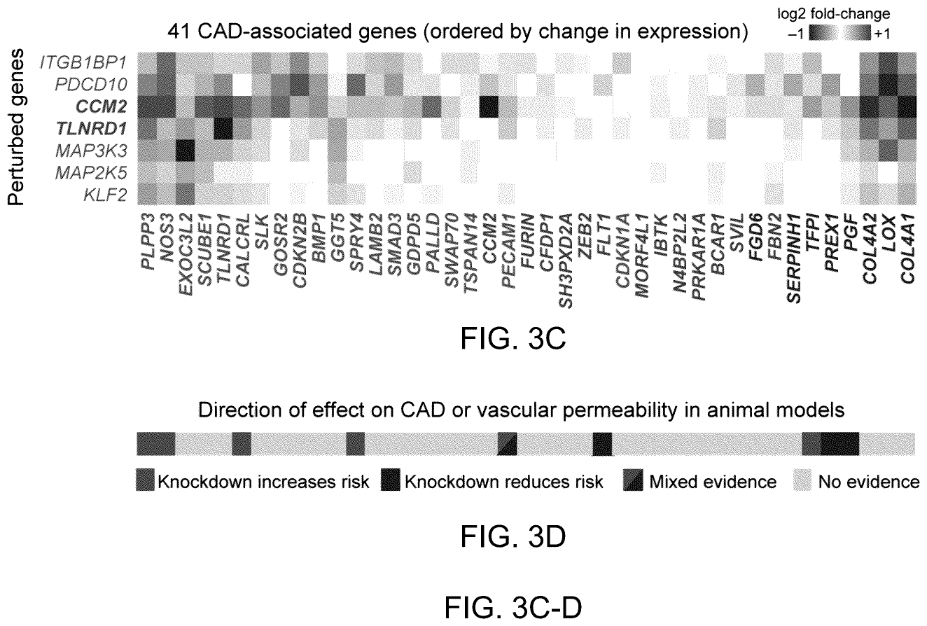

FIG. 3 shows a curated set of genes previously reported to interact physically or functionally with the CCM complex and/or downstream ERK5/MEK5 signaling, plus one additional gene (TLNRD1) identified here as a member of the CCM pathway. These genes were manually selected through an iterative process involving examining genes known to interact with the CCM complex and that were found to regulate the enriched programs in Perturb-seq.

### Allelic Imbalance Analysis for a Variant Linked to TLNRD1

Allelic imbalance in ATAC-seq signal was calculated for the rs1879454 variant, accounting for any mapping bias toward the reference allele following methods previously described. Specifically, two reference genome FASTA files were created that harbored the reference or alternate alleles at rs1879454; ATAC-seq data were aligned to both genome files; reads were selected that overlapped the variant coordinate; PySuspenders and PySAM were used to assign and count reads that aligned to one or the other allele. This procedure was applied to ATAC-seq data from TeloHAEC under control or stimulated conditions and the ENCODE datasets ENCSR000EVW (GATA2 ChIP-seq on human HUVEC) and ENCSR000EOB (DNase-seq and DGF on human HMVEC-dLy-Neo).

### CRISPRi-Flow FISH for TLNRD1

CRISPRi-FlowFISH was used to test the effects of 61 candidate enhancers on TLNRD1 expression in teloHAEC, including the enhancer containing rs1879454. gRNAs tiling across all accessible regions (here, defined as the union of the peaks in the chromatin accessibility dataset called by MACS2 with a lenient P-value cut-off of 0.1, and 150-bp regions on either side of the MACS2 summit) in the range chr15:81,267,614-81,427,246 in ATAC-seq data from TeloHAEC were designed. gRNAs with low specificity scores or low-complexity sequences as previously described were excluded. teloHAECs were infected with the gRNA lentiviral library with 15 ug/mL blasticidin selection for 3 days, and activated CRISPRi with 2 μg/mL doxycycline incubation for 5 days. FlowFISH was performed using ThermoFisher PrimeFlow (ThermoFisher 88-18005-210) as previously described, using ThermoFisher probe sets VA1-3010837-PF for TLNRD1 and VA4-13187-PF for RPL13A. An approximately 2.6-fold signal was observed for TLNRD1 in cells with all probes applied (“stained”) versus cells without target gene probes applied (“unstained”). This data were analyzed as previously described. In brief, gRNAs in each bin were counted using Bowtie to map reads to a custom index, normalized gRNA counts in each bin by library size, then used a maximum-likelihood estimation approach to compute the effect size for each gRNA. The limited-memory Broyden-Fletcher-Goldfarb-Shanno algorithm (implemented in the R stats4 package) was used to estimate the most likely log-normal distribution that would have produced the observed guide counts, and the effect size for each gRNA is the mean of its log-normal fit divided by the average of the means from all negative-control gRNAs. As previously described the effect size of each gRNA in a screen was scaled linearly, so that the strongest 20-guide window at the TSS of the target gene has an 85% effect, in order to account for non-specific probe binding in the RNA FISH assay (this is based on the observation that promoter CRISPRi typically shows 80-90% knockdown by qPCR). The effect sizes of each gRNA across replicates was averaged and the effect size of an element was computed as the average of all gRNAs targeting that element. The significance was assessed using a two-sided t-test comparing the mean effect size of all gRNAs in a candidate element to all negative-control guides. The false-discovery rate (FDR) for elements was computed using the Benjamini-Hochberg procedure and used an FDR threshold of 0.05 to call significant regulatory effects.

### Generation of Single-Guide CRISPRi TeloHAEC Derivatives

Paired oligos for individual guides (newly-designed, or with the best KD efficacy in Perturb-seq) were annealed and cloned into the BsmBI site of the CROP Opti Blast vector, which were then used to generate lentivirus (as per). CRISPRi TeloHAEC were infected with each virus, in separate wells, and selected for blasticidin (15 ug/ml 4 days), before 5 day dox induction and analysis by bulk RNA-seq, fluorescence imaging or physiological assays. Guides (TargetGene_CloneIndex: ForwardSequence) were:

CCM2_C2:
(SEQ ID NO: 14)
GGCAAGAAGGTGAGCGTGCG.
CCM2_F6:
(SEQ ID NO: 4)
GAGCCGCTACATGCTCGACCC.
CDH5_B8:
(SEQ ID NO: 129)
GCCAGCTGGAAAACCTGAAG.
CDH5_D5:
(SEQ ID NO: 139)
GTTGGACTGCCTGTCCGTCCA.
ITGB1BP1_C7:
(SEQ ID NO: 48)
GAAGGCCGCGGCACTCCCACG.
ITGB1BP1_G8:
(SEQ ID NO: 49)
GAAGTCCGCAACCCGGGGAT.
KLF2_C9:
(SEQ ID NO: 200)
GGACCCGGGGAGAAAGGACG.
KLF2_G10:
(SEQ ID NO: 201)
GCCGCGGTATATAAGCCGGC.
MAP2K5 A11:
(SEQ ID NO: 202)
GCCGAGGCCGCGCGGACTGG.
MAP2K5 B5:
(SEQ ID NO: 203)
GTCTGCCCCACCCGGAGACAC.
MAP3K3 A4:
(SEQ ID NO: 204)
GTTCCTGAGGTGGAGAACGG.
MAP3K3 C3:
(SEQ ID NO: 205)
GCCAATAACAAGAAGGAAGT.
MEF2A_C10:
(SEQ ID NO: 206)
GCGGCGCGAAGCGCTGGTGG.
MEF2A H10:
(SEQ ID NO: 207)
GACTGAATTATCCTCTCGGT.
Negative_control_B6:
(SEQ ID NO: 210)
GCAACGGTGTACCGCGGATC.
Negative_control_D2:
(SEQ ID NO: 211)
GTGGTTCACAACCGGACCCA.
Negative_control D8:
(SEQ ID NO: 212)
GGTGGTTCGGTTTGCGTGGCC.
Negative_control F4:
(SEQ ID NO: 213)
GCTGGGCGGACGTTGGGATA.
NFAT5 D4:
(SEQ ID NO: 208)
GGCCTCGCTTCCTGCCGGCG.
NFAT5 D7:
(SEQ ID NO: 209)
GGTCCCCGTCCCGCCGGGGG.
PDCD10 D11:
(SEQ ID NO: 93)
GACCGAGCAGAAGAGGTCTA.
PDCD10 G1:
(SEQ ID NO: 94)
GCCGCTTTACGCCACTCGCGT.
TLNRD1 B3:
(SEQ ID NO: 91)
GTGGCTGCGCCGCCGCCCGCA.
TLNRD1 D12:
(SEQ ID NO: 84)
GCCTCCGGCAGCCCCTGCGGG.

### Ribonucleoprotein-Based CRISPR/Cas9 Genome Editing

For some experiments, Synthego's ribonucleoprotein (RNP) technology was used as an orthologous method to knock down target genes. Briefly, TeloHAEC were nucleofected with Synthego's Gene Knockout Kit v2 for non-targeting negative control, CCM2, or TLNRD1 using the Lonza 4D-Nucleofector system. For each nucleofection reaction, 150,000 cells were used with 20 pmol of Cas9 and 50 pmol of sgRNA. The cells were then nucleofected (program CA-210) using SG cell line nucleofection solution (Lonza; V4XC-3024). The nucleofected cells were seeded in endothelial culture medium, and harvested 48 hours later for RNA extraction for qRT-PCR analysis to measure gene knock down efficiency and perturbation effects. For MAP3K3 knockdown in single-guide CRISPRi lines, cells were treated with 2 μg/ml doxycycline for 72 hours before nucleofection, and for the 48 hours afterwards.

### Computational Prediction of the TLNRD1 and CCM Protein Structure

AlphaFold2.3 Multimer v3 was run using sequences for KRIT1 (UniProt 000522), CCM2 (Uniprot Q9BSQ5, with and without deletion of residues 417-444), PDCD10 (Uniprot Q9BUL8), and TLNRD1 (Uniprot Q9H1K6). Models were visualized using UCSF ChimeraX v1.61. Predicted Alignment Error (PAE) was extracted using AlphaPickle and plotted using combinations of AlphaPickle, Matplotlib v3.7.0, and Seaborn.

### Co-Immunoprecipitation of CCM2 and TLNRD1

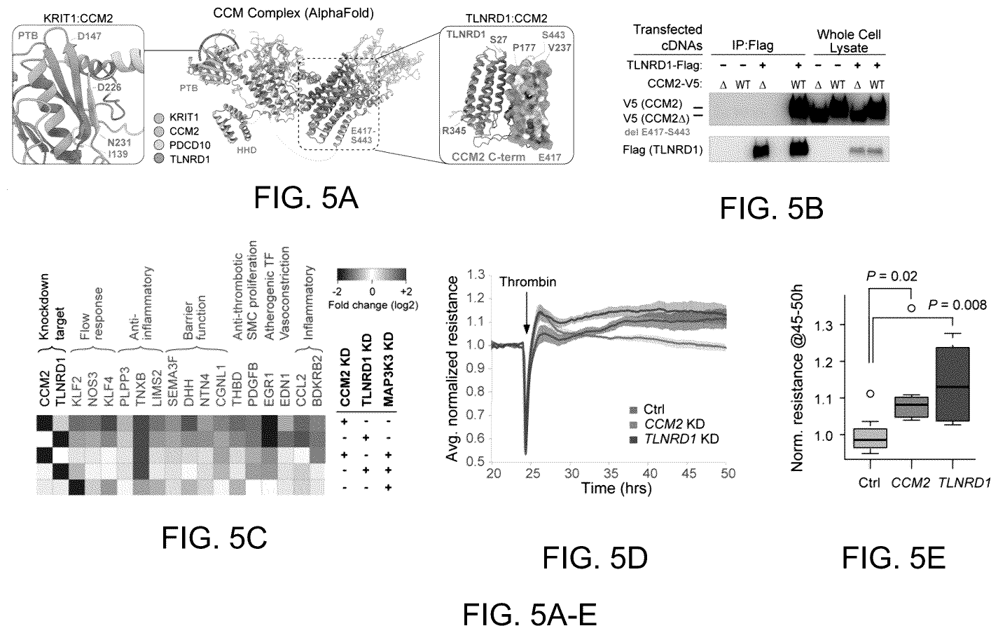

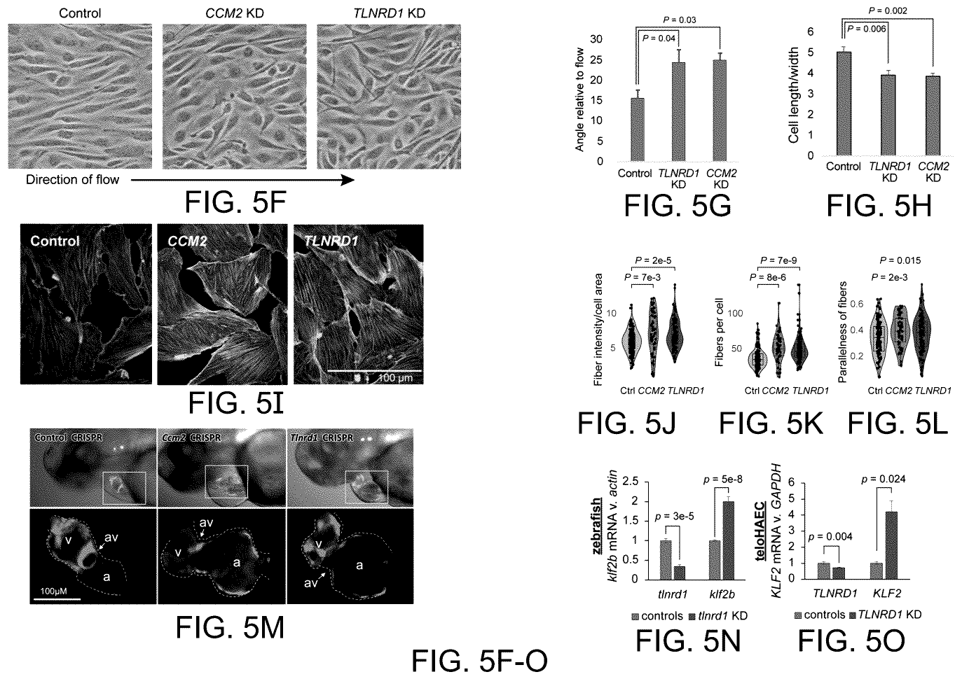

HEK293 cells were transfected with V5-tagged CCM2 full length, V5-tagged CCM2 C-terminal truncation, Flag-tagged TLNRD1 and/or Flag-tagged Akt1 as indicated in FIG. 5B using FuGENE (E2311, Promega). Two days after the transfection, cell lysates were extracted with IP lysis buffer (87787, Thermo Scientific) supplemented with 1× Halt Protease Inhibitor Cocktail (1862209, Thermo Scientific). Immunoprecipitation was carried out using magnetic beads (88805, Thermo Scientific) conjugated with 5 μg of either rabbit anti-V5 (13202, Cell Signaling Technology) or mouse anti-Flag (F1804, Millipore Sigma) antibody. Cell lysates were incubated with the antibody-conjugated beads for 20 mins at room temperature, beads were then washed three times with IP lysis buffer, and precipitants were eluted using 2×LDS sample buffer (NP0007, Thermo Fischer Scientific). Precipitants and total lysates were immunoblotted with anti-V5 (13202, Cell Signaling Technology, ab27671, Abcam), then stripped (21059, Thermo Scientific) and re-probed with anti-Flag (F1804, Millipore Sigma), or vice versa.

### Trans-Endothelial Electrical Resistance (TEER) Measurements

For TEER measurements, the ECIS Z-Theta instrument from Applied BioPhysics was used in the 96-well plate system (Applied BioPhysics; 96W10idf). CRISPRi TeloHAEC expressing individual guides to TLNRD1, CCM2, or non-targeting guides (2 different guides each) were treated for 5 days with 2 ug/ml doxycycline. A gold electrode-containing 96-well ECIS plate was incubated at 37° C. and 5% CO₂ with culture media for 30 min to normalize before coating with 2.5 mg/mL fibronectin in 0.1 M bicarbonate buffer at pH 8.0. Then, the coated wells were inoculated with 45,000 cells in 100 mL media. An additional 100 mL of media was added to each well before initiating the measurements at 4000-Hz ac. At 25 hours, after the cells formed a confluent layer, the culture media was replaced with 200 mL of fresh culture media with 1 U/mL thrombin to disrupt cell-cell junctions, and measurements continued until 50 hrs to observe cell junction recovery post-thrombin treatment.

### Measurement of Endothelial Cell Responses to Laminar Flow

200,000 CRISPRi TeloHAEC cells with individual control, CCM2 or TLNRD1 guides were seeded on flow chamber slides (80176, Ibidi) that had been pre-coated with 0.2% gelatin. After 24 hours, cells were cultured under laminar flow (12 dynes/cm²) for 48 hours (10902, Ibidi pump system). Static culture controls were seeded at the same density. Cells were treated with 2 μg/ml doxycycline for 2 days prior to seeding, and throughout, for a total of 5 days. RNA was harvested with 300 μL of Trizol and extracted with 60 μL of chloroform. After addition of 1 volume 70% ethanol, RNA was loaded onto a Qiagen RNeasy spin column, washed with 350 μL of buffer RW1 and treated for 20 mins at room temperature with 10 μL Purelink DNAse (In Vitrogen 12185010) in 80 μL of 1× buffer. Subsequent RNA purification steps were as per the Qiagen RNeasy protocol.

### Fluorescence Imaging and Quantitation of TeloHAEC

For quantitation of actin fiber characteristics, single guide derivatives of CRISPRi teloHAEC cells were treated with 2 ug/ml doxycycline for 5 days, before fixation in situ with by addition of paraformaldehyde to 3.2% for 30 mins at 37° C., washed with PBS, permeabilized by addition of PBS with 0.1% triton X100 for 15 mins at room temperature, washed with PBS and stained with PerkinElmer Cell Painting dyes (Phenovue Fluor 568-Phalloidin, Phenovue Fluor 488-Concanavalin A, Phenovue Hoechst 33342 Nuclear Stain & Phenovue 512 Nucleic Acid Stain) according to the manufacturer's instructions. Cells were imaged in four channels on a Perkin Elmer Opera Phenix Imaging System-106513, confocal 63× magnification with 1× binning. The stacks of images for the Phalloidin and Hoechst channels were converted to single images using maximum projection, output ranges standardized, and images exported. Cell boundaries were drawn by hand on a Phalloidin/Hoechst composite image in FIJI and saved as regions of interest (ROI). Phalloidin channel images were loaded into FIJI, converted to 16 bit grayscale, and cell areas and dimensions for each ROI were extracted using the Measure function (reporting Area and Fit Ellipse). Actin fibers were detected and quantified using the LPX FIJI plugin, with lineExtract parameters: giwsiter=5, mdnmsLen=8, pickup=above (10.0), shaveLen=3, delLen=5, and line properties for each ROI measured using LineFeature. Parallelness (a_normAvgRad) ranges from 0 (for randomly-oriented fibers) to 1 (all fibers parallel).

### Zebrafish Husbandry and Transgenic Lines

Adult wild type AB, transgenic Tg (flk1:EGFP) (that express EGFP at the surface of blood vessels) and transgenic Tg (cmlc2:EGFP) (that express EGFP in heart muscle) zebrafish lines were maintained at 28.5° C. in circulating system water on a 14-h light/10-h dark cycle under standard conditions. Embryos and larvae (≤5dpf) were kept in the dark in an incubator at 28.5° C. for subsequent experiments. At the end point, embryos were euthanized by tricaine overdose (MS-222; Western Chemical Inc.) followed by freezing (for RNA isolation), PFA-fixing (for histological analysis) or bleach treatment. All animal experiments were performed in accordance with relevant guidelines and regulations and with approval from the Mayo Clinic Institutional Animal Care and Use Committee.

tlnrd1 and ccm2 CRISPR Knockdown in Zebrafish

crRNAs for both ccm2 and tlnrd1 were designed using the Alt-R Predesigned Cas9 crRNA Selection Tool using the Integrated DNA Technologies (IDT) database. All the crRNAs were selected based on published criteria. For ccm2, guides were designed to target two distinct exons shared by all transcripts (AA: TTGAACGGAGACACGATACC (SEQ ID NO: 214), AF: ATGGAGCCACAACACCCACC (SEQ ID NO: 215)). For tlnrd1, guides either targeted the 5′ untranslated region (UTR, AN.1: GGAAACACAAGGGACGTCTC (SEQ ID NO: 216), AF: GCTGAAAGTTACACCCAACG (SEQ ID NO: 217)) or the single tlnrd1 exon (AN.2: CTGCCGCTAAGGATGTTGGT (SEQ ID NO: 218), DG: CAAGAGCAAAATGCAGCTGG (SEQ ID NO: 219)). For ccm2 and tlnrd1, RNPs were prepared as described; briefly, the crRNA (bearing the guide sequence) was annealed with an equal molar amount of tracrRNA (bearing the gRNA scaffold, IDT, #1072532) in duplex buffer (IDT, #11010301), to form gRNA, by heating at 95° C. for 5 min and subsequently cooling on ice. Guide RNA was assembled with an equal molar amount of Alt-R S.p. Cas9 Nuclease V3 (IDT, #1081058) to form the RNP complex (28.5 μM final concentration), by incubation at 37° C. for 5 min followed by storage at −20° C., following the published protocol. RNP complexes prepared from the tracrRNA/scaffold only were used as a negative control. 3 nL of each RNP complex (28.5 μM final concentration) was injected into the yolk of one-to-two cell stage embryos (wildtype, Tg;Fli:EGFP (for the permeability analysis) or Tg;cmlc2:EGFP (for visualization of the atrioventricular valve, AV)).

### Tlnrd1 and Ccm2 Morpholino Knockdown in Zebrafish

Morpholinos (MOs) to knock down tlnrd1 and ccm2 were designed and injected using standard protocols. The ccm2 morpholino has been validated to cause cardiovascular phenotypes at the 100 UM dose. A custom morpholino for tlnrd1 (TTCCCCGAGCCACTACTAGCCATAG (SEQ ID NO: 220)) was designed to target the translation start site and ordered from Gene Tools, LLC. The control oligo is a single sequence, CCTCTTACCTCAGTTACAATTTATA (SEQ ID NO: 221), that is a validated negative control. Wildtype zebrafish embryos were injected with 3 nl of diluted morpholinos at multiple concentrations (50 μM, 100 μM, 200 μM, 300 μM), of control, tlnrd1 and ccm2 morpholinos at one cell stage by using a pico-injector (Harvard Apparatus). For coinjection, tlnrd1 and ccm2 MOs were mixed to give 50 μM of each, and 3 nl of the mixture was injected.

### Zebrafish Imaging and Phenotyping

Embryos were observed for mortality and visible phenotypes at 2 days post-fertilization (dpf) and 3 dpf using a light microscope. Images were captured at 3 dpf on an EVOS microscope (Life technology) and Zeiss Axio-observer Z1. 3 dpf embryos (knock down or control) were scored as having a heart phenotype if they displayed visible atrial chamber enlargement, moderate to severe pericardial edema and slow blood flow in the tail veins. Note that, normal zebrafish undergo cardiac looping between approximately 2dpf and 3dpf (wherein the atrium and ventricle change from a linear posterior-to-anterior arrangement to a right-to-left asymmetric arrangement). Most of the ccm2 or tlnrd1 knockdown embryos that scored positive by the criteria above also showed a looping defect, maintaining the posterior-to-anterior arrangement of atrium and ventricle at 3dpf. However, because looping is a time dependent phenomenon that normally occurs near the 3dpf time when the embryos for heart phenotypes were examined, this was not included as a scoring criterion. For the additional phenotypic analyses described below (confocal imaging, H&E staining, tail vein morphology, blood flow & vascular permeability), ccm2 or tlnrd1 knockdown embryos were selected that scored as positive for heart phenotype at 2dpf. High resolution images for the vascular permeability and cardiac chamber analyses, were acquired using a confocal microscope LSM 800 (Zeiss).

### Histological Staining of Zebrafish Embryos for Atrial/Ventricular Thickness

H&E staining was performed by the Mayo Clinic Comprehensive Cancer Center Histology core lab. Jacksonville, FL. Briefly, zebrafish 3dpf larvae were fixed in 4% paraformaldehyde overnight at 4° C. To obtain paraffin sections, fixed larvae were dehydrated stepwise in ethanol/1×PBS dilutions (5, 25, 50, 75 and 100% ethanol). Transverse sections at a thickness of 5 μm using a microtome (MICROME) were produced from the anterior beginning of the otic vesicle and included posterior structures until the cloacal vent. The sectioned region therefore spanned from the glomerulus up to the cloaca and included the complete pronephros. Sections were stained with Gills 1, eosin Y and Harris hematoxylin (Richard Allan Scientific) according to the manufacturer protocol.

### FITC-Dextran 2000 kDa & Texas Red-Dextran 70 kDa Injections, & Imaging for Tail Vein Morphology and Vascular Permeability

Briefly, at 3-days post-fertilization (3-dpf), Crispr/Cas9-injected embryos were anesthetized in 0.015% tricaine methanesulfonate (Western Chemical, Inc.) and microangiography was performed by inserting a glass microneedle (World precision Instruments, Sarasota, FL) through the pericardium directly into the ventricle. For assessment of vascular morphology, 2000 kDa FITC dextran (Sigma, FD2000S-100 MG) was diluted to 2 mg/ml in Zebrafish embryo medium, and a total of 4.5 nL was injected. For measurement of vascular permeability, Texas Red-dextran with a molecular weight of 70 kDa was solubilized in embryo medium at a 2 mg/mL concentration and a total of 4.5 nL was injected. Images were acquired after 30 minutes, using a Zeiss LSM 880 confocal microscope, and standard FITC and dsRed filter sets, and 10× objective, at room temperature. For quantitation of permeability, the Raw “.czi” images were preprocessed using the Zeiss software (ZEN2) to generate a maximum intensity projection image. The maximum intensity projection images of controls as well as Crispr mutants were then processed using the MATLAB programming platform. Movies for the blood flow in the heart and tail veins were taken by capturing 60 second bright field-time-lapse images at 60 frames per second, using an EVOS microscope at 20× magnification, as described previously.

qRT-PCR Assays in Zebrafish

klf2b, ccm2 & tlnrd1 expression was measured by qRT-PCR on RNA isolated from 100 μM tlnrd1 morpholino embryos at 3 dpf, using primers F:

(SEQ ID NO: 222)
GAAGAGACACCTGTGAGGGC
&
R:
(SEQ ID NO: 223)
GGACACCGATTCGTAGGACC,
for ccm2,
F:
(SEQ ID NO: 224)
GGCGGATCAGATGAGGGAAC
&
R:
(SEQ ID NO: 225)
CAGACAGCAATACGGACCGA,
and
for tlnrd1,
F:
(SEQ ID NO: 226)
ACACGCGAGAGTACCTGTTG
&
R:
(SEQ ID NO: 227)
TCATCCCGCGACAAATCCAA.

### In Situ Hybridization for Tlnrd1 Expression in Zebrafish

In situ hybridization was performed as follows. Briefly, a 437 bp fragment of tlnrd1 was amplified from genomic DNA using the PCR primers, F: CATTAACGGAATGGCAGGCG (SEQ ID NO: 228) and R: TGCCCGGATAAAGGCAAAGT (SEQ ID NO: 229), subcloned and verified by sequencing. Antisense in situ hybridization probes were generated using an M13 reverse primer with SpeI-linearized plasmid, while sense (negative control) probes were generated using an M13 forward primer with NotI-linearized plasmid. In situ hybridization of embryos was conducted at 24 and 72 hrs post-fertilization using these anti-sense or sense (control) probes against tlnrd1.

### Applying the Variant-to-Gene-to-Program Approach to Additional GWAS Traits and Cell Types

It was tested whether the V2G2P method was generally applicable to other traits beyond CAD in endothelial cells, and to other cell types.

First, it was examined whether the same Perturb-seq dataset in endothelial cells could be applied to interpret variants for other vascular traits related to endothelial cell functions, beyond CAD. V2G2P was applied to 2 additional GWAS traits (Pulse Pressure (PP) and Mean Arterial Pressure (MAP), from the UK Biobank, with finemapping information from Hilary Finucane and Jacob Ulirsch: www.finucanelab.org/data). V2G analysis was performed by mapping variants associated with these traits onto the same endothelial cell enhancer map used for CAD, and identified genes linked to PP or MAP variants in endothelial cells. V2G2P analysis was then performed, by testing for enrichment of the PP or MAP V2G gene sets in the 50 endothelial cell programs identified from Perturb-seq. Note, that the V2G2P enrichment test was performed using only the 300 co-regulated genes in each program, because not all the genes at GWAS loci for these blood pressure traits were targeted for perturbation in the endothelial cell Perturb-seq screen.

Next, it was examined whether the entire analysis framework could be applied to another cell type: K562 erythroid cells, which are a relevant model for red blood cell and platelet traits. Here, 7 GWAS traits for red blood cell and platelet measures were examined: Mean Corpuscular Hemoglobin (MCH), Mean Corpuscular Volume (MCV), Platelet Count (Plt), Red Blood Cell count (RBC), Mean Corpuscular Hemoglobin Concentration (MCHC), Hemoglobin A1c (HbA1c) and Hemoglobin (Hb), along with 4 traits for which K562 cells are not likely to be an appropriate model: pulse pressure (PP), mean arterial pressure (MAP), systolic blood pressure (SBP) & diastolic blood pressure (DBP), from the UK biobank, with finemapping by Hilary Finucane and Jacob Ulirsch: www.finucanelab.org/data.

V2G maps were constructed for each trait using ABC data in K562 cells (K562-Roadmap), to identify variant-containing enhancers, and identified the set of V2G genes for each trait (genes with links to variants associated, by GWAS, with each trait). We, then, constructed a gene-to-program map by applying cNMF to the genome-scale Perturb-seq data previously collected in K562 cells. K values were tested over a broad range, and selected K=90 as the number of components that minimized cNMF error and maximized other ranking metrics (see “Choosing the number of components for cNMF analysis” above). Finally, the V2G2P enrichment test (considering both the 300 co-regulated genes for each program and the regulators of each program, identified as the perturbations significantly affecting expression of each program, from Perturb-seq) were performed. Of the 90 programs, 32 programs were found to be prioritized for at least one of 6 GWAS traits.

### Curating Previously-Identified CAD Prioritization Gene Sets

To assess the ability of V2G2P to prioritize disease-associated genes, several CAD studies that used more than just GWAS and genomic positioning data to prioritize CAD loci and genes were surveyed. Below is a summary of how each study created their gene set and how this data was accessed.

Aragam et al., polygenic prioritization score (PoPS): Computed POPS score for all protein-coding genes within 500 kb of all GWAS signals and prioritized the gene with the highest POPS score in each locus, resulting in 221 genes.

Hodonsky et al., eQTL and sQTL colocalization: Bulk RNA-seq was collected from human coronary artery tissue samples from explanted transplant tissue, or collected from rejected transplant donors (138 individuals, from left anterior descending coronary artery, right coronary artery, and left circumflex artery). eQTL colocalization was performed to find eQTL-associated genes (eGenes), or to find splice QTLs (sQTLs). The eQTL list was from Supplementary Table 12 column “vdh_CAD_PPH4” with posterior probability >0.8 (Methods: “PPH4>0.8 to support evidence of a shared causal variant”). The slice variant list was from Supplementary Table 22 and subset for variants with posterior probability >0.8 (column “vdh_CAD_PPH4”). sGenes linked to the colocalized sQTLs were then identified by finding matching genes in Supplementary Table 20 (columns “gene_id” and “spliceid”).

Li et al., transcriptome-wide association study (TWAS): Associated genotype and expression data across 15 tissues (7 from STARNET and 8 from GTEx). Supplementary Table 4 for significant TWAS genes (114 genes) was used.

OpenTarget L2G: Used a supervised machine-learning model to learn the weights of multiple evidence sources (distance, molecular QTL colocalization, chromatin interaction, and variant pathogenicity) based on a gold standard of previously identified causal genes. The authors applied this model to the van der Harst coronary artery disease GWAS dataset. Prioritized genes had an L2G model score >0.5 (table downloaded from https://genetics.opentargets.org/Study/GCST005194/associations).

Stolze et al., endothelial cell-specific eQTL colocalization: Human aortic endothelial cells (HAECs) were isolated from deceased heart donor aortic trimmings and cultured+/−IL-1beta (53 individuals, bulk RNA-seq), as well as 157 EC donors' cultured ECs+/−oxPL treatment (microarray). They performed eQTL mapping using Matrix eQTL and used the R package “coloc” for colocalization. Their data was obtained from Table S5.

van der Harst and Verweij: Prioritized variants using Probabilistic Annotation Integrator based on several features such as LD information, p-value distribution, coding genes, and H3K4me1 sites. Data were obtained from Table 2 and Online Table XX.

Wunnemann et al., endothelial cell CRISPR screen for 6 phenotypes: The authors used a CRISPR screening approach to identify CAD risk variant-containing regulatory elements in 83 CAD GWAS loci that altered FACS-sortable signals for any of 6 pre-selected phenotypes in endothelial cells (E-selectin, ICAM1, VCAM1, nitric oxide, reactive oxygen species, and intracellular calcium). The identified 26 loci where perturbation of a variant-containing element affected one or more of these phenotypes (prioritizing a single gene in 21 of these loci). Data was obtained from their FIG. 3A and Supplementary Table 4.

### Example 2. A Variant-to-Gene Map in Endothelial Cells

To address various challenges described herein, including the challenge of identifying which genes work together in which pathways in which cell types, a 5-step Variant-to-Gene-to-Program (V2G2P) approach (FIG. 1A) was developed. The 5 steps of the developed approach are as follows:
1. Identifying a cell type and cellular model relevant to disease genetics, through enrichment of disease risk variants in enhancers in that cell type—To accomplish this, epigenomic data was analyzed to prioritize telomerase-immortalized human aortic endothelial cells (teloHAEC) to study the role of endothelial cells in coronary artery disease;
2. Building a map of variant-to-gene (V2G) links in that cell type, to link disease-associated variants to potential target genes—To accomplish this, evidence from variants in endothelial cell enhancers, as well as coding regions and splice sites, was examined;
3. Building a map of gene-to-program (G2P) links in that cell type, by using Perturb-seq to systematically knock down all possible candidate disease genes and identify sets of genes that act together in biological pathways—All expressed genes within 500 kb of CAD GWAS loci were knocked down, and the effects of each perturbation were read out with single cell RNA-seq;
4. Identifying “disease-associated programs”, by developing a statistical test to determine whether the genes with links to risk variants are enriched in (converge on) particular programs—This step revealed that many CAD GWAS loci converge on 5 gene programs identified de novo with Perturb-seq, which appear to correspond to branches of the cerebral cavernous malformations (CCM) signal transduction pathway, whose potential role in coronary artery disease risk had not yet been characterized; and
5. Studying the genes in disease-associated programs—In this step, 41 genes were nominated as likely to influence CAD risk through effects in endothelial cells. One new gene, TLNRD1, was dissected in detail, and this gene was observed to be a novel regulator in the CCM pathway. The 41 nominated genes are listed below in Table 2.

TABLE 2
Nominated CAD-associated genes
Nominated CAD-associated Genes

| | | | |
|---|---|---|---|
| BCAR1 | FGD6 | NOS3 | SMAD3 |
| BMP1 | FLT1 | PALLD | SPRY4 |
| CALCRL | FURIN | PECAM1 | SVIL |
| CCM2 | GDPD5 | PGF | SWAP70 |
| CDKN1A | GGT5 | PLPP3 | TFPI |
| CDKN2B | GOSR2 | PREX1 | TLNRD1 |
| CFDP1 | IBTK | PRKAR1A | TSPAN14 |
| COL4A1 | LAMB2 | SCUBE1 | ZEB2 |
| COL4A2 | LOX | SERPINH1 | |
| EXOC3L2 | MORF4L1 | SH3PXD2A | |
| FBN2 | N4BP2L2 | SLK | |

In summary, V2G2P defined cell-type specific programs de novo using Perturb-seq, combined these programs with enhancer-to-gene maps from the same cell type, and provided an interpretable framework for tracing the path from variant to gene to disease program at individual GWAS loci. This framework expanded on recent findings that combining V2G and G2P evidence can identify causal genes with improved precision.

A V2G2P approach was implemented to study the role of endothelial cells in CAD risk (FIG. 1A). GWAS signals for CAD from recent meta-analyses (see Aragam, K. G. et al. Discovery and systematic characterization of risk variants and genes for coronary artery disease in over a million participants. bioRxiv (2021) doi: 10.1101/2021.05.24.21257377; Harst, P. van der, van der Harst, P. & Verweij, N. Identification of 64 Novel Genetic Loci Provides an Expanded View on the Genetic Architecture of Coronary Artery Disease. Circulation Research vol. 122 433-443 Preprint at doi.org/10.1161/circresaha.117.312086 (2018)), were collected, and a set of “nearby genes” for each GWAS signal was defined to include the 2 closest genes on either side, plus all genes within 500 Kb. The 228 “non-lipid” GWAS signals that were not associated with circulating lipid levels were focused on in this study (see Methods), because lipid-associated signals likely act in hepatocytes or other non-endothelial cell types. Altogether, this yielded 1,942 total candidate genes, with a median of 8 nearby genes per GWAS signal.

Telomerase-immortalized primary human aortic endothelial cells (teloHAEC) were selected as a cellular model and bulk RNA-seq, ATAC-seq, and H3K27ac ChIP-seq data was collected in resting and several stimulated conditions (+IL1β, TNFα, VEGFA) to identify expressed genes and candidate enhancers. Variants in teloHAEC enhancers were 11-to-13-fold enriched for CAD heritability by stratified linkage disequilibrium score regression, and the genes near CAD GWAS loci that were expressed in teloHAEC were also expressed in primary coronary artery endothelial cells in vivo, supporting the choice of this cellular model.

To link risk variants to genes (V2G), variants for each GWAS signal, and variants to genes were linked based on that variant (i) being in a coding sequence or splice site, or (ii) overlapping an enhancer linked to the gene by the Activity-by-Contact model (ABC, which performs well, and better than other methods, at linking noncoding variants to target genes in specific cell types), considering the 2 genes per signal with the strongest ABC scores; or (iii) a variant overlapping a chromatin accessible region close to a gene, considering the 2 closest genes to the variant. Enhancers identified in resting and stimulated teloHAEC were included, as well as other previous datasets, to capture many possible endothelial cell states. 254 of 1,942 nearby genes with a link to a CAD risk variant (“genes with a V2G link”, or simply “V2G genes”) were identified at 125 of 228 non-lipid GWAS signals (range: 1-5 genes per signal). At most GWAS signals, multiple nearby genes had V2G links, because there were multiple candidate variants per signal and/or because individual noncoding variants were linked to more than one gene, consistent with previous observations.

### Example 3. A Gene-to-Program Map in Endothelial Cells

To link genes to programs (G2P), Perturb-seq was applied together with a matrix factorization approach to identify de novo sets of genes which are co-regulated in TeloHAEC. Information about the perturbations that significantly alter expression of these co-regulated gene sets was then integrated to define a catalog of 50 gene “programs”. Each program includes 300-335 genes discovered and grouped in an unbiased fashion (FIG. 1B).

Data were generated to identify co-regulated gene programs using CRISPRi Perturb-seq in teloHAEC targeting the promoters of 2,285 genes, including all 1,661 expressed nearby genes around lipid and non-lipid CAD GWAS signals, as well as 624 control genes (e.g., genes known to regulate endothelial functions, or genes not expressed in TeloHAEC). 15 guides were cloned per promoter plus 1,000 non-targeting and safe-targeting guides, for a total of 37,637 guides, into a modified CROP-seq vector. TeloHAEC was engineered to express KRAB-dCas9 (CRISPR interference (CRISPRi)) under a doxycycline-inducible promoter. Cells were transduced with the guide library at a low multiplicity of infection. Cells were harvested 5 days after activation of CRISPRi expression. 20 lanes of 10×3′ single-cell RNA-seq were collected.

In total, data for 214,449 cells expressing a single guide were obtained, at an average depth of 10,870 transcriptome-mapped reads with distinct unique molecular identifiers (UMIs) per cell. This dataset included on average 5.7 cells per guide, 85.5 cells per target promoter, and 929,000 total transcript UMIs per targeted promoter. It was found that target genes were effectively knocked down (FIG. 1C), that knock down of common essential genes decreased fitness (FIG. 1D), and that 14% of perturbations of expressed targets significantly impacted the transcriptome.

An unsupervised approach was applied to this Perturb-seq data to discover gene programs independent of previous knowledge of annotated pathways or gene sets (FIG. 1B, bottom). First, consensus non-negative matrix factorization (cNMF) was used to model the gene-by-cell matrix as a linear combination of latent components in which each component has a non-negative weight for each gene and has non-negative expression in each cell. 50 components were identified, after optimizing the number of components and excluding components correlated with batch.

From each component from cNMF, a “program” was defined: a set of genes comprised of both “co-regulated genes” (the 300 marker genes whose expression is most specific to that component as described herein) and “regulators” (those genes whose perturbations significantly affected the expression of a component compared to negative control guide RNAs (gRNAs), with experiment-wide FDR<0.05). This analysis defined 50 programs, each including 300 co-regulated genes and from 0 to 35 regulators (300 to 335 total genes per program). Together, this gene-to-program map included 18,606 links from 7,692 unique genes to the 50 programs (FIG. 1E).

After defining the 50 programs using an unsupervised approach independent of any prior information about gene sets or pathways, each program was annotated based on their regulators and co-regulated genes (including manual curation, analysis of transcription factor (TF) motifs in their promoters and predicted enhancers, and gene set enrichment) Programs were identified representing an array of cellular functions: from ubiquitously expressed (“housekeeping”) processes, inducible stress responses, to pathways specifically associated with endothelial cells (FIG. 1E). 13 programs were annotated as “endothelial-cell-specific” because they included genes that were on average more highly expressed in endothelial cells than in other cell types. These endothelial-cell-specific programs included distinct combinations of genes enriched for roles in angiogenesis, extracellular matrix remodeling, barrier function, and the endothelial-to-mesenchymal transition (endoMT), and the promoters of their co-regulated genes were enriched for different transcription factor motifs (FIG. 1F). Notably, although the Perturb-seq experiment is conducted in resting, unstimulated conditions, several programs were identified that are related to specific stimulus responses. These included non-cell type-specific programs for unfolded protein response (UPR), DNA damage, heat shock, and inflammation, as well as endothelial-specific programs. For example, Program 15 (Flow response, KLF2) appeared to correspond to a canonical endothelial cell response to laminar shear stress defined by the known flow-responsive transcription factor KLF2: the program was highly enriched for KLF motifs in promoters; included known flow-responsive genes such as KRT18/19, NOS3, and KLF2 itself; and was significantly reduced by perturbations to MAP2K5 (MEK5), a kinase known to act in the signaling pathway upstream of KLF2 (FIG. 1F).

Analysis of regulators (perturbations) showed expected effects on programs related to known metabolic or regulatory pathways, and identified cases where programs were coordinately or oppositely regulated by the same perturbations. 31 regulators significantly regulated 5 or more programs, and 10 perturbed genes regulated 5 or more endothelial-cell specific programs, including genes known to have important functions in endothelial cells such as EGFL7 and ITGB1BP1/ICAP1.

Taken together, this gene-to-program map represented a wide range of cellular pathways, links upstream regulators to coherent sets of downstream genes, and provided a resource for understanding the functions and potential disease-relevance of genes in endothelial cells. Using this gene-to-program map from Perturb-seq, the 228 non-lipid CAD GWAS signals were annotated, and 883 of 1,942 nearby genes were found to be linked to at least one program, and that all 50 programs included one or more genes near CAD GWAS signals.

### Example 4. CAD GWAS Signals Converge on 5 Gene Programs

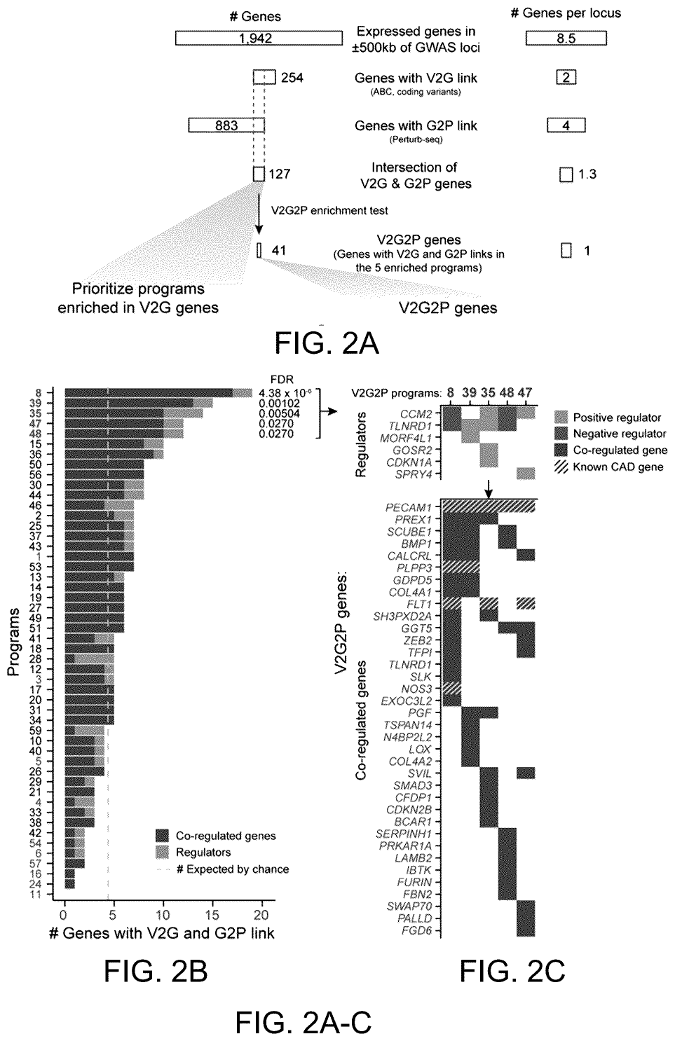

Next, a simple statistical test (“V2G2P enrichment”) was developed to determine, in an unbiased fashion, whether GWAS variants for a trait would converge onto particular gene programs. Specifically, it was evaluated whether genes for each program (Genes with a G2P link, FIG. 2A) were more highly enriched in genes likely to be affected by CAD risk variants (Genes with a V2G link, FIG. 2A) than expected by chance. To account for the differing numbers of possible program regulators (drawn from the 2,285 perturbed genes) and co-regulated genes (drawn from all expressed genes), Fisher's exact tests conducted for program regulators and co-regulated genes were separated and the p-values were combined to create a single score for each program (V2G2P enrichment test, FIG. 2A).

Significant V2G2P enrichment was identified for 5 programs: Program 8 (regulation of angiogenesis & osmotic balance), 39 (basement membrane & platelet recruitment), 35 (focal adhesions, JUN), 47 (angiogenesis, GATA2), and 48 (calcium-dependent cell adhesion) (FIG. 2A). Each of these V2G2P programs included 12 to 18 genes linked to CAD variants, versus 4.5 expected by chance (2.6- to 4-fold enrichment, FDR<0.05, FIG. 2B). Together, these 5 programs included 41 unique V2G2P genes (genes linked to CAD variants and part of at least one of the 5 V2G2P programs), including genes near 43 of 228 non-lipid GWAS signals (FIG. 2C).

Several independent lines of evidence supported the associations of these 5 programs and 41 genes with CAD. (i) All 5 V2G2P programs were endothelial cell programs that included at least 1 of the 8 known genes whose variant-to-gene-to-disease effects in endothelial cells have previously been characterized (“known endothelial cell CAD genes: in FIG. 2C). Program 8 included 4 such genes: NOS3, PLPP3, FLT1, and PECAM1. (ii) All 5 V2G2P programs were significantly enriched for CAD heritability by MAGMA, and two were significantly enriched for CAD heritability by S-LDSC. (iii) The 41 V2G2P genes were highly ranked by an independent gene prioritization method, PoPS, compared to other nearby genes at the same GWAS signals. (iv) 9 of the 41 V2G2P genes have previously been found to affect atherosclerosis and/or vascular barrier integrity via studies in mouse models, in a way that is consistent with their acting in endothelial cells.

Of the 41 V2G2P genes, 4 were known endothelial cell CAD genes, 7 have been previously nominated as likely causal CAD genes with roles in endothelial cells, and 23 have been nominated as likely causal for CAD through approaches that do not specify a relevant cell type. Altogether, the 41 V2G2P genes included 31 genes not previously suggested to affect CAD risk through effects in ECs, and 17 genes not previously linked to CAD risk through any cell type.

Several observations were made that helped to explain the ability of V2G2P analysis to identify disease-associated genes and programs (FIG. 2A). Most notably, at most GWAS signals, neither V2G nor G2P information alone was sufficient to identify likely disease genes: 119 GWAS signals had 2 or more genes with a V2G link (up to 5), and 195 GWAS signals had 2 or more genes with a G2P link (up to 25), including links to all 50 programs. These observations were consistent with the expectation that noncoding variants often regulate multiple nearby genes, and that, by chance, a given GWAS signal might have several nearby genes involved in various cellular pathways. Combining these two layers of information in the V2G2P enrichment test provided far more specificity: for the 43 signals with V2G2P links to these programs, only 6 had more than 1 linked gene (up to 2). This finding further supported other observations that combining locus-specific variant-to-gene links with genome-wide enrichments for gene pathways can improve the specificity of disease gene identification.

In summary, the V2G2P analysis supported that CAD GWAS signals converge on specific gene programs associated with disease in endothelial cells.

### Example 5. The Cerebral Cavernous Malformations Pathway Regulates CAD-Associated Programs

Remarkably, all 5 V2G2P programs for CAD were regulated in Perturb-seq by the same V2G2P gene-cerebral cavernous malformations 2 (CCM2)—and/or by other genes known to act in the same pathway (FIG. 2C, FIG. 3A and FIG. 3B).

In the variant-to-gene analysis, CCM2 harbored a missense coding variant (rs2107732) that has been associated with a decreased risk of CAD in multiple recent GWASs (odds ratio: 0.92, P=1.53×10⁻⁸) (see e.g., Aragam, K. G. et al. Discovery and systematic characterization of risk variants and genes for coronary artery disease in over a million participants. bioRxiv (2021) doi: 10.1101/2021.05.24.21257377). rs2107732 was the lead variant for this GWAS signal, and lead to a valine-to-isoleucine substitution in CCM2 at amino acid 74—in the PTB domain that is responsible for CCM2 interactions with KRIT1.

In Perturb-seq, knock down of CCM2 regulated 4 of the 5 V2G2P programs at an experiment-wide FDR<0.05, and the fifth was nominally significant (P<0.05) (−28% to +90% effects on program expression; FIG. 3B). Other genes known to interact physically or functionally with CCM2 showed directionally concordant effects on the V2G2Pprograms, including KRIT1, VE-cadherin (CDH5), integrin B1 binding protein 1 (ITGB1BP1), alpha catenin (CTNNA1), and heart of glass 1 (HEG1) (FIG. 3A and FIG. 3B). As expected, knock down of genes known to be repressed by CCM2—including MEK5 (MAP2K5), ERK5 (MAPK7), and KLF4—affected the expression of these gene programs in the opposite direction (FIG. 3A and FIG. 3B).

Downstream of the CCM pathway, the V2G2Pprograms for CAD corresponded to distinct sets of genes related to extracellular matrix (ECM) organization, cell migration, and angiogenesis—all processes that have been observed to change upon inactivation of the CCM complex and that may have roles in atherosclerosis (FIG. 3A and FIG. 3B). Program 8 included genes involved in negative regulation of angiogenesis (IGFBP4 and IGFBP5) and osmotic balance (SLC12A2 and AQP1), and included 4 CAD genes known to function in endothelial cells, including NOS3 and PLPP3, which are protective against atherosclerosis. Program 48 included genes involved in cell adhesion and migration such as FSLT1 and TIMP2, and was regulated by MEK5/MAP2K5, ERK5/MAPK7, and calcium/calmodulin-dependent (CAMKK2) signaling. Program 39 expressed genes involved in the basement membrane (COL4A1/2) and platelet recruitment (VWF, SELP). Program 35 expressed genes involved in focal adhesions (ITGA2) and the JAK/STAT signaling pathway. Program 47 expressed genes involved in angiogenesis including NR2F2 and NRP1/2, including two genes specifically associated with a stalk cell phenotype (VWF, EHD4).

To further characterize the co-regulation of the 41 V2G2P genes for CAD, 6 genes in the CCM pathway (ITGB1BP1, CCM2, PDCD10, MAP3K3, MAP2K5 and KLF2) were individually knocked down and gene expression changes were subsequently measured using bulk RNA-seq. 28 of the 41 V2G2P genes were significantly differentially expressed upon CCM pathway perturbation (FDR<0.05, FIG. 3C, Table 2). 8 of these 28 genes have previously been studied in mice to characterize their functions in endothelial cells on atherosclerosis and or vascular permeability, allowing for the assessment of how changes in gene expression downstream of the CCM complex might relate to disease phenotypes in vivo. The direction of effect on disease phenotypes and response to CCM2 knock down were similar (FIG. 3D): of the 5 genes previously shown to maintain vascular barrier function or be protective for atherosclerosis, 4 (NOS3, PLPP3, CALCRL, and SPRY4) were up-regulated in response to CCM2 knock down, whereas genes previously shown to promote atherosclerosis or vascular dysfunction (PGF and PREX1) were down-regulated. One additional gene (PECAM1) has been observed to have mixed directions of effect on disease depending on the genetic model (FIG. 3D). Thus, down-regulation of the CCM complex led to changes in gene expression that may be protective for CAD. Interestingly, this is opposite of the direction of effect of the CCM complex on cerebral cavernous malformations, where loss-of-function mutations increase risk.

These findings implicated the CCM complex, together with its associated regulators and downstream transcriptional effects, in genetic risk for CAD via effects in endothelial cells.

### Example 6. TLNRD1: From Association to Function at 15q25.1

Among the 17 novel TLNRD1: genes for CAD, TLNRD1 (talin rod domain containing 1), a poorly studied gene near the 15q25.1 CAD GWAS signal, was analyzed in greater detail. TLNRD1 was previously found to interact with F-actin and to affect cell migration in a cancer cell line, but has not previously been linked to coronary artery disease or any function in endothelial cells. Notably, however, TLNRD1 was the gene whose knockdown in Perturb-seq most strongly regulated the 5 V2G2P programs for CAD (FIG. 2C, FIG. 3B, and FIG. 4A). To gain further clues about its potential function in endothelial cells and relationship to CAD, the Perturb-seq dataset was further examined, and it was found that the effects of TLNRD1 knockdown on program expression were extremely similar to the effects of CCM2 knockdown (FIG. 4B). Accordingly, it was explored whether TLNRD1 might be a CAD risk gene that acts in the CCM pathway.

The risk variants in the 15q25.1 locus were associated with CAD (lead variant P=2.63×10⁻¹⁰; rank: 159 of 241) but not with lipid levels or blood pressure, and were located in an intergenic region between CFAP161 (the closest gene) and TLNRD1 (FIG. 4C). Based on V2G2P analysis, TLNRD1 was the only gene in the locus with both a variant-to-gene and a gene-to-program link.

To determine whether a variant in this locus might indeed regulate TLNRD1 expression in endothelial cells, the epigenomic datasets were examined using CRISPRi-FlowFISH to perturb candidate enhancers near TLNRD1. It was found that a chromatin accessible element containing rs1879454 (hg19 chr15:81377717: C (major, risk allele)→A (minor, protective allele); MAF=0.16) indeed regulated TLNRD1 expression (estimated—21% effect, FDR corrected two-sided Student's t-test, P=0.001, FIG. 4D, FIG. 4E). rs1879454 also appeared to affect the regulatory activity of this element. The A allele was predicted to disrupt a GATA motif, and, in cells heterozygous for this variant, the A allele was associated with a 2-fold decrease in allele-specific GATA2 ChIP-seq signal in HUVEC (binomial P=0.0758) and a 2.4-fold decrease in allele-specific ATAC-seq signal in teloHAEC (binomial P=0.0058) (FIG. 4F and FIG. 4G).

Next, the functional relationship between TLNRD1 and CCM2 was investigated. CCM2 is known to physically interact with other proteins in the CCM complex and downstream pathways. A recent yeast-2-hybrid screen provided preliminary evidence of a direct interaction between CCM2 and TLNRD1. AlphaFold2.3 Multimer was used to model potential interactions between the three core CCM proteins and TLNRD1, and it was found that TLNRD1 was predicted to directly bind the C-terminal helix of CCM2 (C-helix, residues 417-443, FIG. 5A, right inset), as part of a consistent high confidence arrangement that also recapitulated the known CCM2/KRIT1 binding site in the PDB domain of CCM2 (FIG. 5A, left inset). The CCM2/TLNRD1 interaction depends on the C-helix of CCM2, which binds the TLNRD1 nine-helix bundle (FIG. 5A). The TLNRD1-CCM2 interaction was tested in human cells and a co-immunoprecipitation analysis was performed. It was observed that TLNRD1 immunoprecipitated with CCM2 pulldown, and vice versa, but that this interaction was lost upon deletion of the C-helix of CCM2 (FIG. 5B).

It was noted that perturbation of TLNRD1 or CCM2 had similar effects on V2G2P programs and genes, while perturbations of downstream signaling genes (MAP3K3, MAP2K5, MAPK7, KLF2 & KLF4) had the opposing direction of effect (FIG. 3). For CCM2, this is consistent with its known role in inhibiting MAP3K3/MEKK3 signaling. To test if the transcriptional effects of TLNRD1 knockdown were also related to MAP3K3 signaling, these genes were knocked down in combination. It was observed that knockdown of TLNRD1 or CCM2 upregulated KLF2, KLF4, NOS3 and other likely atheroprotective genes and downregulated likely atherogenic genes, that MAP3K3 knockdown had the opposite effect, and that double knockdown of MAP3K3 and TLNRD1 or MAP3K3 and CCM2 had intermediate effects on these genes (FIG. 5C). Applicant observed similar effects when considering all regulated genes. These observations are consistent with the expected role of the CCM complex in inhibiting the MAP3K3/MEKK3 signaling cascade, and the similarities between CCM2 and TLNRD1 in these effects support that both genes are required for CCM complex signaling.

Knockdown of CCM2 in endothelial cells in vitro has previously been observed to lead to upregulation of KLF signaling, re-organization of the actin cytoskeleton, altered responses to laminar flow, and changes in barrier function—all endothelial cell processes that are thought to be relevant to atherosclerosis in vivo. Similar tests for TLNRD1 were conducted, and it was observed that CRISPRi knock down of either TLNRD1 or CCM2 in teloHAEC indeed led to increased expression of KLF2, NOS3, PLPP3 and other genes likely to be protective for atherosclerosis (FIG. 5C), and also increased endothelial cell barrier function, as measured by trans-endothelial electrical resistance (FIG. 5D and FIG. 5E). Interestingly, when cells under static conditions were compared to cells under laminar shear stress (“flow”, 12 dynes/cm²), the transcriptional effects of TLNRD1 or CCM2 knockdown in static culture were similar to the effects of flow on control cells (Pearson R=0.40, P=1.5e-54 for CCM2; R=0.52, P=1.6e-94 for TLNRD1). TLNRD1 or CCM2 knockdown cells showed reduced alignment to flow, relative to control cells (FIG. 5F-5H), and a weaker transcriptional response to flow, apparently because the flow-response program was already partly active. Consistent with similar transcriptional effects of flow and either TLNRD1 or CCM2 knockdown, it was observed that CCM2 or TLNRD1 knockdown in static culture increased the number and parallelness of actin stress fibers (FIG. 5I-5L), a characteristic of flow response in unperturbed ECs, consistent with prior studies of CCM2 knockdown in HUVEC. Together, these observations indicate that TLNRD1 and CCM2 play very similar roles in endothelial cell phenotypes relevant to CAD, and suggest that down-regulation of TLNRD1 or CCM2 may be atheroprotective by conferring a “flow-like” response and improving barrier function in endothelial cells not exposed to laminar flow, which are most prone to atherogenesis.

To determine whether TLNRD1 has an evolutionarily conserved role in the CCM pathway, TLNRD1 expression and function was assessed in zebrafish. Consistent with TLNRD1 being most highly expressed in human endothelial cells (Tabula Sapiens, atlas of gene expression), tlnrd1 expression was strong in the dorsal vasculature as well as the brain and heart. Several studies have identified a role for CCM2 in heart and vascular development in zebrafish. Similar experiments were performed here, and it was found that knock down of either tlnrd1 or ccm2 with CRISPR and found highly similar effects on cardiac and vascular development, including atrial chamber enlargement, pericardial edema and atrioventricular valve defects (FIG. 5M). Similar effects were also observed using 100 μM tlnrd1 or ccm2 morpholinos. CRISPR-targeted embryos for both tlnrd1 and ccm2 also had thin ventricular walls, and vascular defects, including posterior cardinal vein (PCV) dilation and increased vascular permeability to red dextran particles. Additionally, TLNRD1 knock down led to increased KLF2B expression, similar to the effect of human TLNRD1 knock down on KLF2B expression in teloHAECs (FIG. 5N and FIG. 5O). Finally, it was observed that a 50 μM dose of either tlnrd1 or ccm2 morpholino alone had no effect on cardiovascular morphology, 50 M of both morpholinos had similar effects as the 100 μM dose of either morpholino alone, consistent with both proteins functioning in the same pathway. These data show that tlnrd1 phenocopies ccm2 in zebrafish, indicating deep evolutionary conservation of the functional relationship between these two genes.

Together, these data indicate that TLNRD1 is a strong candidate CAD gene, regulates phenotypes relevant to CAD in endothelial cells in vitro, and appears to be a previously unrecognized regulator in the CCM pathway.

### Example 7. Application to Therapy

In the studies described herein, 35 variants of CCM pathway associated genes were identified that have an effect on endothelial cell function. These variants were compared to a lipid-specific polygenic risk score.

In a clinical trial for a lipid-lowering agent (Evolocumab), it was observed that individuals with elevated endothelial cell-associated polygenic risk score benefited significantly from aggressive LDL-C lowering therapy, whereas individuals who did not have an elevated endothelial cell-associated polygenic risk score did not achieve the same benefit. In this way, an endothelial cell-specific polygenic risk score was able to predict the likelihood of a subject responding favorably to an LDL-C lowering therapy. Accordingly, the detection of one or more loss-of-function variants of CCM pathway associated genes, as described herein, may be predictive of the likelihood of a subject responding favorably to an endothelial cell-specific therapy for a vascular disease (e.g., CAD), such as, but not limited to the gene therapies described herein.

The results described herein suggest a model for how certain CAD risk variants tune endothelial cell functions to influence risk for CAD. In particular, variants affecting CCM2 and TLNRD1 may down-regulate CCM complex signaling, and thereby alter the expression of many other genes linked to CAD risk variants, including up-regulation of the atheroprotective factors NOS3, PLPP3, and other genes downstream of KLF2/4, and down-regulation of PREX1, PGF, and other candidate disease-promoting genes (e.g., FIG. 3 and FIG. 5C). These changes in gene expression may help protect against atherosclerosis by mimicking responses induced by laminar flow and improving barrier function in arterial endothelial cells (FIGS. 5D, 5E, and 5I-5L), and also by acting in a non-cell-autonomous fashion on other cell types in the vessel wall (e.g., by production of nitric oxide by NOS3). Notably, this newly discovered role for the CCM complex in CAD (where partial down-regulation appears to be protective) differs from its previously known role in cerebral cavernous malformations (where complete loss-of-function is thought to be pathogenic). The functions of the many newly identified CAD-associated genes present opportunities for therapeutic interventions.

### Example 8. Exemplary sgRNA-Guided Reduction in Target Gene Expression

sgRNAs of the present disclosure are capable of targeting a gene editing system to target locus, resulting in modulation of expression of a gene of the locus. For example, sgRNAs described herein have been shown to be capable of targeting a gene editing system to a target locus containing a target gene, and reducing target gene expression in cells, relative to that of cells that were not targeted by the gene editing system. Examples of such sgRNAs include: SEQ ID NO: 4 (−4.369), SEQ ID NO: 14 (−2.590), SEQ ID NO: 48 (−2.883), SEQ ID NO: 49 (−2.710), SEQ ID NO: 84 (−2.201), SEQ ID NO: 91 (−3.384), SEQ ID NO: 93 (−2.814), SEQ ID NO: 94 (−1.193), SEQ ID NO: 129 (−5.191), and SEQ ID NO: 139 (−5.340). The number in parentheses for each sgRNA refers to the magnitude of the change in expression of RNA transcripts of the given gene in arterial endothelial cells targeted by a gene editing system comprising an sgRNA comprising a given sequence, relative to that of cells that were not targeted by the gene editing system. The (−) indicates a reduction in target gene expression.

These examples are provided for illustrative purposes only and not to limit the scope of the claims provided herein.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof, inclusive of the endpoints. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art, all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

What is claimed is:

1. An engineered, non-naturally occurring gene editing system comprising:
a) a single guide RNA (sgRNA) comprising a guide sequence capable of hybridizing with a target sequence, or a polynucleotide encoding the sgRNA, and
b) an effector protein, or one or more nucleotide sequences encoding the effector protein;
wherein the sgRNA hybridizes to said target sequence, and the sgRNA forms a complex with the effector protein;
wherein the effector protein comprises a nuclease and/or an effector domain,
wherein the sgRNA is capable of hybridizing to one or more target genes, and
wherein the one or more target gene is a gene of the Cerebral Cavernous Malformation (CCM) pathway or a gene that regulates the CCM pathway.

2. The engineered, non-naturally occurring gene editing system of claim 1, wherein the one or more target genes are selected from the group consisting of: TLNRD1, CCM2, HEG1, ITGB1BP1, KRIT1, PDCD10, ARPC2, CDC42, CDH5, DNM2, MEAF6, PDCD7, RHOA, KLF2, KLF4, MAP2K5, MAP3K3, MEF2A, and NFAT5.

3. The engineered, non-naturally occurring gene editing system of claim 1, wherein the sgRNA comprises a nucleotide sequence as set forth in any one of SEQ ID NO: 3-209.

4. The engineered, non-naturally occurring gene editing system of claim 1, wherein the gene editing system comprises a Clustered Regularly Interspaced Short Palindromic Repeats (CRISPR)-associated (CRISPR-Cas) system.

5. The engineered, non-naturally occurring gene editing system of claim 1, wherein the effector protein comprises a Cas protein fused to a repression domain.

6. An adeno-associated virus (AAV) particle comprising the gene editing system of claim 1.

7. An isolated cell comprising the gene editing system of claim 1.

8. A method for treating a vascular disease in a subject comprising:
administering to the subject a therapeutically effective amount of a pharmacological agent capable of modulating the expression of a target gene in vascular endothelial cells,
wherein the target gene is a Cerebral Cavernous Malformation (CCM) pathway gene or a gene that regulates the function of the CCM pathway.

9. The method of claim 8, wherein the vascular disease is coronary artery disease (CAD).

10. The method of claim 8, wherein the subject has, is suspected of having, or is at risk for developing the vascular disease.

11. The method of claim 8, wherein the target gene is selected from the group consisting of: TLNRD1, CCM2, HEG1, ITGB1BP1, KRIT1, PDCD10, ARPC2, CDC42, CDH5, DNM2, MEAF6, PDCD7, RHOA, KLF2, KLF4, MAP2K5, MAP3K3, MEF2A, and NFAT5.

12. The method of claim 8, wherein the pharmacological agent is a gene editing system.

13. The method of claim 8, wherein the gene editing system comprises a Clustered Regularly Interspaced Short Palindromic Repeats (CRISPR)-associated (CRISPR-Cas) system, comprising:
a) a single guide RNA (sgRNA) comprising a guide sequence capable of hybridizing with the target sequence, or a polynucleotide encoding the sgRNA, and
b) an effector protein, or one or more nucleotide sequences encoding the effector protein;
wherein the sgRNA hybridizes to the target sequence, and the sgRNA forms a complex with the effector protein;
wherein the effector protein comprises a nuclease and/or an effector domain; and
wherein the sgRNA is capable of hybridizing to one or more genes selected from the group consisting of: TLNRD1, CCM2, HEG1, ITGB1BP1, KRIT1, PDCD10, ARPC2, CDC42, CDH5, DNM2, MEAF6, PDCD7, RHOA, KLF2, KLF4, MAP2K5, MAP3K3, MEF2A, and NFAT5.

14. The method of claim 8, wherein the sgRNA comprises a nucleotide sequence as set forth in any one of SEQ ID NO: 3-209.

15. The method of claim 8, further comprising:
administering to the subject a therapeutically effective amount of a pharmacological agent capable of increasing the activity of MEKK3, MEK5, ERK5, KLF2, or KLF4 in vascular endothelial cells of the subject.

16. A method for determining whether a subject having, suspected of having, or at risk for a vascular disease is likely to respond to a therapy for the vascular disease, comprising:
(a) analyzing a biological sample obtained from the subject, wherein the biological sample comprises nucleic acids;
(b) detecting the presence or absence of one or more nucleic acid sequence variant that results in loss-of-function of one or more genes of the Cerebral Cavernous Malformation (CCM) pathway or one or more genes that regulate the CCM pathway; and
(c) determining that the subject is more likely to respond to the therapy if the one or more nucleic acid sequence variant is detected.

17. The method of claim 16, wherein the therapy comprises:
administering to the subject a therapeutically effective amount of a pharmacological agent capable of reducing the expression of a target gene in vascular endothelial cells,
wherein the target gene is a Cerebral Cavernous Malformation (CCM) pathway gene or a gene that regulates the function of the CCM pathway.

18. The method of claim 17, wherein the target gene is selected from the group consisting of TLNRD1, CCM2, HEG1, ITGB1BP1, KRIT1, PDCD10, ARPC2, CDC42, CDH5, DNM2, MEAF6, PDCD7, RHOA, KLF2, KLF4, MAP2K5, MAP3K3, MEF2A, and NFAT5.

19. The method of claim 16, further comprising:
(d) determining that the subject is less likely to respond to the therapy if the one or more nucleic acid sequence variant is not detected.

20. A method for modifying a target locus of interest, the method comprising:
delivering to the target locus the gene editing system of claim 1,
wherein the effector protein forms a complex with the sgRNA and upon binding of the complex to the target locus of interest, the effector protein induces a modification of the target locus of interest, and
wherein the target locus of interest is within a cell.


SEQUENCE LISTING (236 sequences)

SEQ ID NO: 1 - protein, 465 aa, Homo sapiens
MHSSCRQRRNQNLSKEIPQTEFHTGYSMENEPGIVSPFKRVFLKGEKSRDKKAHEKVTERRPLHTVVLSLPERVEPDRLLSDYIEKEVKYLGQLTSIPGYLNPSSRTEILHFIDNAKRAHQLPGHLTQEHDAVLSLSAYNVKLAWRDGEDIILRVPIHDIAAVSYVRDDAAHLVVLKTAQDPGISPSQSLCAESSRGLSAGSLSESAVGPVEACCLVILAAESKVAAEELCCLLGQVFQVVYTESTIDFLDRAIFDGASTPTHHLSLHSDDSSTKVDIKETYEVEASTFCFPESVDVGGASPHSKTISESELSASATELLQDYMLTLRTKLSSQEIQQFAALLHEYRNGASIHEFCINLRQLYGDSRKFLLLGLRPFIPEKDSQHFENFLETIGVKDGRGIITDSFGRHRRALSTTSSSTTNGNRATGSSDDRSAPSEGDEWDRMISDISSDIEALGCSMDQDSA

SEQ ID NO: 2 - protein, 362 aa, Homo sapiens
MASGSAGKPTGEAASPAPASAIGGASSQPRKRLVSVCDHCKGKMQLVADLLLLSSEARPVLFEGPASSGAGAESFEQCRDTIIARTKGLSILTHDVQSQLNMGRFGEAGDSLVELGDLVVSLTECSAHAAYLAAVATPGAQPAQPGLVDRYRVTRCRHEVEQGCAVLRATPLADMTPQLLLEVSQGLSRNLKFLTDACALASDKSRDRFSREQFKLGVKCMSTSASALLACVREVKVAPSELARSRCALFSGPLVQAVSALVGFATEPQFLGRAAAVSAEGKAVQTAILGGAMSVVSACVLLTQCLRDLAQHPDGGAKMSDHRERLRNSACAVSEGCTLLSQALRERSSPRTLPPVNSNSVN

SEQ ID NO: 3 - DNA, 21 bp, synthetic construct
  source 1..21: mol_type = other DNA
gagacttcgggcccggctggc

SEQ ID NO: 4 - DNA, 21 bp, synthetic construct
  source 1..21: mol_type = other DNA
gagccgctacatgctcgaccc

SEQ ID NO: 5 - DNA, 21 bp, synthetic construct
  source 1..21: mol_type = other DNA
gatcgccgcgtgcggctcccg

SEQ ID NO: 6 - DNA, 21 bp, synthetic construct
  source 1..21: mol_type = other DNA
gatgagcgacaaccgtgggcg

SEQ ID NO: 7 - DNA, 21 bp, synthetic construct
  source 1..21: mol_type = other DNA
gcaagaaggtgagcgtgcgcg

SEQ ID NO: 8 - DNA, 21 bp, synthetic construct
  source 1..21: mol_type = other DNA
gcatgtagcggctgctggcgg

SEQ ID NO: 9 - DNA, 21 bp, synthetic construct
  source 1..21: mol_type = other DNA
gcgcggcgatatggaagagga

SEQ ID NO: 10 - DNA, 20 bp, synthetic construct
  source 1..20: mol_type = other DNA
gcggagacttcgggcccggc

SEQ ID NO: 11 - DNA, 21 bp, synthetic construct
  source 1..21: mol_type = other DNA
gctcttccatatcgccgcgtg

SEQ ID NO: 12 - DNA, 20 bp, synthetic construct
  source 1..20: mol_type = other DNA
gctgctggcggcggggctcc

SEQ ID NO: 13 - DNA, 20 bp, synthetic construct
  source 1..20: mol_type = other DNA
ggatgagcgacaaccgtggg

SEQ ID NO: 14 - DNA, 20 bp, synthetic construct
  source 1..20: mol_type = other DNA
ggcaagaaggtgagcgtgcg

SEQ ID NO: 15 - DNA, 20 bp, synthetic construct
  source 1..20: mol_type = other DNA
ggccccaccttccccagcgc

SEQ ID NO: 16 - DNA, 20 bp, synthetic construct
  source 1..20: mol_type = other DNA
gtcgagcatgtagcggctgc

SEQ ID NO: 17 - DNA, 20 bp, synthetic construct
  source 1..20: mol_type = other DNA
gtgaggatgagcgacaaccg

SEQ ID NO: 18 - DNA, 20 bp, synthetic construct
  source 1..20: mol_type = other DNA
gaagtcggccgccgtaaaga

SEQ ID NO: 19 - DNA, 21 bp, synthetic construct
  source 1..21: mol_type = other DNA
gagcctgcctcgcgtccaggg

SEQ ID NO: 20 - DNA, 21 bp, synthetic construct
  source 1..21: mol_type = other DNA
gatttgccgccatctttacgg

SEQ ID NO: 21 - DNA, 21 bp, synthetic construct
  source 1..21: mol_type = other DNA
gcaggtctctgaaaaggggac

SEQ ID NO: 22 - DNA, 21 bp, synthetic construct
  source 1..21: mol_type = other DNA
gcccgcgtccttcaggagagc

SEQ ID NO: 23 - DNA, 21 bp, synthetic construct
  source 1..21: mol_type = other DNA
gcggccgtgcagaagcgaagt

SEQ ID NO: 24 - DNA, 21 bp, synthetic construct
  source 1..21: mol_type = other DNA
gcgtccagggaggcggagctc

SEQ ID NO: 25 - DNA, 21 bp, synthetic construct
  source 1..21: mol_type = other DNA
gctcctgaaggacgcggggcg

SEQ ID NO: 26 - DNA, 20 bp, synthetic construct
  source 1..20: mol_type = other DNA
gctgagtgggcggagcgaac

SEQ ID NO: 27 - DNA, 21 bp, synthetic construct
  source 1..21: mol_type = other DNA
gctgcctcgcgtccagggagg

SEQ ID NO: 28 - DNA, 20 bp, synthetic construct
  source 1..20: mol_type = other DNA
ggagctcaggtctctgaaaa

SEQ ID NO: 29 - DNA, 21 bp, synthetic construct
  source 1..21: mol_type = other DNA
gtcccgcgcgcgctgagtggg

SEQ ID NO: 30 - DNA, 21 bp, synthetic construct
  source 1..21: mol_type = other DNA
gtccggctctcctgaaggacg

SEQ ID NO: 31 - DNA, 20 bp, synthetic construct
  source 1..20: mol_type = other DNA
gtctctgaaaaggggacggg

SEQ ID NO: 32 - DNA, 21 bp, synthetic construct
  source 1..21: mol_type = other DNA
gtgcagaagcgaagtaggggt

SEQ ID NO: 33 - DNA, 21 bp, synthetic construct
  source 1..21: mol_type = other DNA
gaagttatcgggagccgcggg

SEQ ID NO: 34 - DNA, 21 bp, synthetic construct
  source 1..21: mol_type = other DNA
gaggctcagcgcgcggcgagc

SEQ ID NO: 35 - DNA, 21 bp, synthetic construct
  source 1..21: mol_type = other DNA
gagttatcgggagccgcggga

SEQ ID NO: 36 - DNA, 21 bp, synthetic construct
  source 1..21: mol_type = other DNA
gcccgcccgcgctcacatgcc

SEQ ID NO: 37 - DNA, 21 bp, synthetic construct
  source 1..21: mol_type = other DNA
gcctcactccgctcacttccc

SEQ ID NO: 38 - DNA, 20 bp, synthetic construct
  source 1..20: mol_type = other DNA
gcgcgccgggcatgtgagcg

SEQ ID NO: 39 - DNA, 21 bp, synthetic construct
  source 1..21: mol_type = other DNA
gcgcgccgggcatgtgagcgc

SEQ ID NO: 40 - DNA, 20 bp, synthetic construct
  source 1..20: mol_type = other DNA
gcgcggcgaggccatggtga

SEQ ID NO: 41 - DNA, 20 bp, synthetic construct
  source 1..20: mol_type = other DNA
gcgctcacatgcccggcgcg

SEQ ID NO: 42 - DNA, 20 bp, synthetic construct
  source 1..20: mol_type = other DNA
gcgggacccgccgccttccc

SEQ ID NO: 43 - DNA, 20 bp, synthetic construct
  source 1..20: mol_type = other DNA
gctcacatgcccggcgcgcg

SEQ ID NO: 44 - DNA, 20 bp, synthetic construct
  source 1..20: mol_type = other DNA
gctcagcgcgcggcgagccg

SEQ ID NO: 45 - DNA, 20 bp, synthetic construct
  source 1..20: mol_type = other DNA
ggctcagcgcgcggcgagcc

SEQ ID NO: 46 - DNA, 21 bp, synthetic construct
  source 1..21: mol_type = other DNA
gtcaaaagttatcgggagccg

SEQ ID NO: 47 - DNA, 21 bp, synthetic construct
  source 1..21: mol_type = other DNA
gtggcctcgccgcgcgcctcg

SEQ ID NO: 48 - DNA, 21 bp, synthetic construct
  source 1..21: mol_type = other DNA
gaaggccgcggcactcccacg

SEQ ID NO: 49 - DNA, 20 bp, synthetic construct
  source 1..20: mol_type = other DNA
gaagtccgcaacccggggat

SEQ ID NO: 50 - DNA, 20 bp, synthetic construct
  source 1..20: mol_type = other DNA
gaccccgaagtccgcaaccc

SEQ ID NO: 51 - DNA, 21 bp, synthetic construct
  source 1..21: mol_type = other DNA
gacttggggtcttggaaccgg

SEQ ID NO: 52 - DNA, 20 bp, synthetic construct
  source 1..20: mol_type = other DNA
gagcacaacttccgtcactc

SEQ ID NO: 53 - DNA, 21 bp, synthetic construct
  source 1..21: mol_type = other DNA
gagtagctgcctgggcgccaa

SEQ ID NO: 54 - DNA, 20 bp, synthetic construct
  source 1..20: mol_type = other DNA
gagtctagtgccgcgggctc

SEQ ID NO: 55 - DNA, 21 bp, synthetic construct
  source 1..21: mol_type = other DNA
gccacttggggtcttggaacc

SEQ ID NO: 56 - DNA, 20 bp, synthetic construct
  source 1..20: mol_type = other DNA
gccctcctgcccacgtccgc

SEQ ID NO: 57 - DNA, 21 bp, synthetic construct
  source 1..21: mol_type = other DNA
gcctcccctcgacttccggtc

SEQ ID NO: 58 - DNA, 21 bp, synthetic construct
  source 1..21: mol_type = other DNA
gcgcagccgcgggcccatccc

SEQ ID NO: 59 - DNA, 21 bp, synthetic construct
  source 1..21: mol_type = other DNA
gtcccgttcccgctccagcgc

SEQ ID NO: 60 - DNA, 21 bp, synthetic construct
  source 1..21: mol_type = other DNA
gtgcgcatagtcttggaggcg

SEQ ID NO: 61 - DNA, 21 bp, synthetic construct
  source 1..21: mol_type = other DNA
gtggaaaagccggcgctggag

SEQ ID NO: 62 - DNA, 20 bp, synthetic construct
  source 1..20: mol_type = other DNA
gtggtgggaagcctcgcgag

SEQ ID NO: 63 - DNA, 21 bp, synthetic construct
  source 1..21: mol_type = other DNA
gaaggacagtaaagagcaggt

SEQ ID NO: 64 - DNA, 21 bp, synthetic construct
  source 1..21: mol_type = other DNA
gagagcttccgggtcggcggc

SEQ ID NO: 65 - DNA, 20 bp, synthetic construct
  source 1..20: mol_type = other DNA
gagcaggaacggcagcagcc

SEQ ID NO: 66 - DNA, 21 bp, synthetic construct
  source 1..21: mol_type = other DNA
gagggaaggacagtaaagagc

SEQ ID NO: 67 - DNA, 21 bp, synthetic construct
  source 1..21: mol_type = other DNA
gcacccgcgccccaaaagctc

SEQ ID NO: 68 - DNA, 21 bp, synthetic construct
  source 1..21: mol_type = other DNA
gcaccctctcccgaaactacg

SEQ ID NO: 69 - DNA, 20 bp, synthetic construct
  source 1..20: mol_type = other DNA
gccaaccgcgaggctgcaaa

SEQ ID NO: 70 - DNA, 21 bp, synthetic construct
  source 1..21: mol_type = other DNA
gcgatttcatagccacggtaa

SEQ ID NO: 71 - DNA, 21 bp, synthetic construct
  source 1..21: mol_type = other DNA
gcggtgaaagagcttccgggt

SEQ ID NO: 72 - DNA, 20 bp, synthetic construct
  source 1..20: mol_type = other DNA
gctaaggcctttgaagacct

SEQ ID NO: 73 - DNA, 20 bp, synthetic construct
  source 1..20: mol_type = other DNA
gtagtttcgggagagggtga

SEQ ID NO: 74 - DNA, 21 bp, synthetic construct
  source 1..21: mol_type = other DNA
gtcacaccctttgcagcctcg

SEQ ID NO: 75 - DNA, 21 bp, synthetic construct
  source 1..21: mol_type = other DNA
gtcccgaaactacggggtccc

SEQ ID NO: 76 - DNA, 21 bp, synthetic construct
  source 1..21: mol_type = other DNA
gttcatagccacggtaacggc

SEQ ID NO: 77 - DNA, 21 bp, synthetic construct
  source 1..21: mol_type = other DNA
gtttccaccccgccgttaccg

SEQ ID NO: 78 - DNA, 21 bp, synthetic construct
  source 1..21: mol_type = other DNA
gaaaagatttctataggctcc

SEQ ID NO: 79 - DNA, 20 bp, synthetic construct
  source 1..20: mol_type = other DNA
gaaactgaaaggaagggcgg

SEQ ID NO: 80 - DNA, 21 bp, synthetic construct
  source 1..21: mol_type = other DNA
gaaagcctccggcagcccctg

SEQ ID NO: 81 - DNA, 21 bp, synthetic construct
  source 1..21: mol_type = other DNA
gagaaactgaaaggaagggcg

SEQ ID NO: 82 - DNA, 20 bp, synthetic construct
  source 1..20: mol_type = other DNA
gatttctataggctccaggg

SEQ ID NO: 83 - DNA, 20 bp, synthetic construct
  source 1..20: mol_type = other DNA
gccgcgcgcggagaggaagc

SEQ ID NO: 84 - DNA, 21 bp, synthetic construct
  source 1..21: mol_type = other DNA
gcctccggcagcccctgcggg

SEQ ID NO: 85 - DNA, 20 bp, synthetic construct
  source 1..20: mol_type = other DNA
gcgggcagaaactgaaagga

SEQ ID NO: 86 - DNA, 21 bp, synthetic construct
  source 1..21: mol_type = other DNA
gctccagggaggttacggccg

SEQ ID NO: 87 - DNA, 21 bp, synthetic construct
  source 1..21: mol_type = other DNA
gctccgaggtgaagccgcgcg

SEQ ID NO: 88 - DNA, 20 bp, synthetic construct
  source 1..20: mol_type = other DNA
ggaagcgggatttaaacgaa

SEQ ID NO: 89 - DNA, 21 bp, synthetic construct
  source 1..21: mol_type = other DNA
gtcagtttctgcccgctcgct

SEQ ID NO: 90 - DNA, 21 bp, synthetic construct
  source 1..21: mol_type = other DNA
gtcccgtccgcgcctcgcccc

SEQ ID NO: 91 - DNA, 21 bp, synthetic construct
  source 1..21: mol_type = other DNA
gtggctgcgccgccgcccgca

SEQ ID NO: 92 - DNA, 21 bp, synthetic construct
  source 1..21: mol_type = other DNA
gttcacctcggagcgcggccg

SEQ ID NO: 93 - DNA, 20 bp, synthetic construct
  source 1..20: mol_type = other DNA
gaccgagcagaagaggtcta

SEQ ID NO: 94 - DNA, 21 bp, synthetic construct
  source 1..21: mol_type = other DNA
gccgctttacgccactcgcgt

SEQ ID NO: 95 - DNA, 20 bp, synthetic construct
  source 1..20: mol_type = other DNA
gaaacaagggccggtgaggg

SEQ ID NO: 96 - DNA, 20 bp, synthetic construct
  source 1..20: mol_type = other DNA
gaagggtaagacgaacgcgc

SEQ ID NO: 97 - DNA, 21 bp, synthetic construct
  source 1..21: mol_type = other DNA
gagccgccgccatgatcctgc

SEQ ID NO: 98 - DNA, 21 bp, synthetic construct
  source 1..21: mol_type = other DNA
gagttcgagaacgcggccgcc

SEQ ID NO: 99 - DNA, 21 bp, synthetic construct
  source 1..21: mol_type = other DNA
gcacctccagcaggatcatgg

SEQ ID NO: 100 - DNA, 21 bp, synthetic construct
  source 1..21: mol_type = other DNA
gccccacccagccccactcag

SEQ ID NO: 101 - DNA, 21 bp, synthetic construct
  source 1..21: mol_type = other DNA
gccccaggggaaggagaaaca

SEQ ID NO: 102 - DNA, 21 bp, synthetic construct
  source 1..21: mol_type = other DNA
gcctcccttacccaccctcac

SEQ ID NO: 103 - DNA, 21 bp, synthetic construct
  source 1..21: mol_type = other DNA
gcgagcgtctcctcgatgatg

SEQ ID NO: 104 - DNA, 21 bp, synthetic construct
  source 1..21: mol_type = other DNA
gcgcgctcaagttcgagaacg

SEQ ID NO: 105 - DNA, 21 bp, synthetic construct
  source 1..21: mol_type = other DNA
gctccagcaggatcatggcgg

SEQ ID NO: 106 - DNA, 20 bp, synthetic construct
  source 1..20: mol_type = other DNA
ggagaaacaagggccggtga

SEQ ID NO: 107 - DNA, 20 bp, synthetic construct
  source 1..20: mol_type = other DNA
ggggaaggagaaacaagggc

SEQ ID NO: 108 - DNA, 21 bp, synthetic construct
  source 1..21: mol_type = other DNA
gtaccccacccagccccactc

SEQ ID NO: 109 - DNA, 21 bp, synthetic construct
  source 1..21: mol_type = other DNA
gtgcggttgttcacctccagc

SEQ ID NO: 110 - DNA, 21 bp, synthetic construct
  source 1..21: mol_type = other DNA
gagttgggtgcccgcggaagt

SEQ ID NO: 111 - DNA, 21 bp, synthetic construct
  source 1..21: mol_type = other DNA
gcccgcctgtcgcgcacgcgc

SEQ ID NO: 112 - DNA, 20 bp, synthetic construct
  source 1..20: mol_type = other DNA
gcccgcgcgtgcgcgacagg

SEQ ID NO: 113 - DNA, 21 bp, synthetic construct
  source 1..21: mol_type = other DNA
gcctgttcctcccacttccgc

SEQ ID NO: 114 - DNA, 20 bp, synthetic construct
  source 1..20: mol_type = other DNA
gcgcgcaggagacgcacagt

SEQ ID NO: 115 - DNA, 21 bp, synthetic construct
  source 1..21: mol_type = other DNA
gcggggcccgaggttccccgc

SEQ ID NO: 116 - DNA, 20 bp, synthetic construct
  source 1..20: mol_type = other DNA
ggagaagctgaggtgagtgc

SEQ ID NO: 117 - DNA, 20 bp, synthetic construct
  source 1..20: mol_type = other DNA
ggcgttggcagcactgcgcg

SEQ ID NO: 118 - DNA, 20 bp, synthetic construct
  source 1..20: mol_type = other DNA
gggcggagccagggcagtcc

SEQ ID NO: 119 - DNA, 20 bp, synthetic construct
  source 1..20: mol_type = other DNA
ggggcgttggcagcactgcg

SEQ ID NO: 120 - DNA, 20 bp, synthetic construct
  source 1..20: mol_type = other DNA
gtagggtggcagctcccgcg

SEQ ID NO: 121 - DNA, 21 bp, synthetic construct
  source 1..21: mol_type = other DNA
gtcacctcagcttctccaccg

SEQ ID NO: 122 - DNA, 21 bp, synthetic construct
  source 1..21: mol_type = other DNA
gtccgtcctctcccagtcctc

SEQ ID NO: 123 - DNA, 20 bp, synthetic construct
  source 1..20: mol_type = other DNA
gtctcctcggctgccggaca

SEQ ID NO: 124 - DNA, 20 bp, synthetic construct
  source 1..20: mol_type = other DNA
gtgcccctgtccggcagccg

SEQ ID NO: 125 - DNA, 21 bp, synthetic construct
  source 1..21: mol_type = other DNA
gagccagcccagccctcacaa

SEQ ID NO: 126 - DNA, 21 bp, synthetic construct
  source 1..21: mol_type = other DNA
gagccaggcctccctcgccag

SEQ ID NO: 127 - DNA, 21 bp, synthetic construct
  source 1..21: mol_type = other DNA
gcaataacaggaaaccatccc

SEQ ID NO: 128 - DNA, 21 bp, synthetic construct
  source 1..21: mol_type = other DNA
gcatccctcagcccacaggca

SEQ ID NO: 129 - DNA, 20 bp, synthetic construct
  source 1..20: mol_type = other DNA
gccagctggaaaacctgaag

SEQ ID NO: 130 - DNA, 21 bp, synthetic construct
  source 1..21: mol_type = other DNA
gcccacaggcacggtgagtgg

SEQ ID NO: 131 - DNA, 21 bp, synthetic construct
  source 1..21: mol_type = other DNA
gccccgctggcgagggaggcc

SEQ ID NO: 132 - DNA, 21 bp, synthetic construct
  source 1..21: mol_type = other DNA
gcgtccagggctgagcgtgag

SEQ ID NO: 133 - DNA, 21 bp, synthetic construct
  source 1..21: mol_type = other DNA
gctcaccgtgcctgtgggctg

SEQ ID NO: 134 - DNA, 21 bp, synthetic construct
  source 1..21: mol_type = other DNA
gctgcagataggcaggccctg

SEQ ID NO: 135 - DNA, 20 bp, synthetic construct
  source 1..20: mol_type = other DNA
ggagccacaccccgctggcg

SEQ ID NO: 136 - DNA, 20 bp, synthetic construct
  source 1..20: mol_type = other DNA
ggctcccctccaaagacggt

SEQ ID NO: 137 - DNA, 21 bp, synthetic construct
  source 1..21: mol_type = other DNA
gtaacaggaaaccatcccagg

SEQ ID NO: 138 - DNA, 20 bp, synthetic construct
  source 1..20: mol_type = other DNA
gtcagccgaccgtctttgga

SEQ ID NO: 139 - DNA, 21 bp, synthetic construct
  source 1..21: mol_type = other DNA
gttggactgcctgtccgtcca

SEQ ID NO: 140 - DNA, 21 bp, synthetic construct
  source 1..21: mol_type = other DNA
gacccgagcgacctcccgccc

SEQ ID NO: 141 - DNA, 20 bp, synthetic construct
  source 1..20: mol_type = other DNA
gagcgcagggcgctcgggcc

SEQ ID NO: 142 - DNA, 20 bp, synthetic construct
  source 1..20: mol_type = other DNA
gcaacggctacagacgccgc

SEQ ID NO: 143 - DNA, 21 bp, synthetic construct
  source 1..21: mol_type = other DNA
gccctcaacgacctggccccg

SEQ ID NO: 144 - DNA, 20 bp, synthetic construct
  source 1..20: mol_type = other DNA
gccgccgaccctcaacgacc

SEQ ID NO: 145 - DNA, 20 bp, synthetic construct
  source 1..20: mol_type = other DNA
gccgttgctccccgcccgcc

SEQ ID NO: 146 - DNA, 21 bp, synthetic construct
  source 1..21: mol_type = other DNA
gcgggcgtcttgccgaggccc

SEQ ID NO: 147 - DNA, 21 bp, synthetic construct
  source 1..21: mol_type = other DNA
gcgtcttgccgaggcccgggc

SEQ ID NO: 148 - DNA, 21 bp, synthetic construct
  source 1..21: mol_type = other DNA
gctcacggtcgccgcctcatc

SEQ ID NO: 149 - DNA, 21 bp, synthetic construct
  source 1..21: mol_type = other DNA
gctcggcaagacgcccgctcc

SEQ ID NO: 150 - DNA, 20 bp, synthetic construct
  source 1..20: mol_type = other DNA
ggcgaccgtgaggccgagcc

SEQ ID NO: 151 - DNA, 21 bp, synthetic construct
  source 1..21: mol_type = other DNA
gtatccgagtggcggtgcagc

SEQ ID NO: 152 - DNA, 21 bp, synthetic construct
  source 1..21: mol_type = other DNA
gtattggctagtgtatccgag

SEQ ID NO: 153 - DNA, 21 bp, synthetic construct
  source 1..21: mol_type = other DNA
gtgcagcaggccagtcgcgtg

SEQ ID NO: 154 - DNA, 21 bp, synthetic construct
  source 1..21: mol_type = other DNA
gtgtcgcctgagaaccggatg

SEQ ID NO: 155 - DNA, 20 bp, synthetic construct
  source 1..20: mol_type = other DNA
gacaggggttgcacggcatg

SEQ ID NO: 156 - DNA, 21 bp, synthetic construct
  source 1..21: mol_type = other DNA
gatctctagagctctccgaga

SEQ ID NO: 157 - DNA, 20 bp, synthetic construct
  source 1..20: mol_type = other DNA
gccccggccttagcgaagct

SEQ ID NO: 158 - DNA, 21 bp, synthetic construct
  source 1..21: mol_type = other DNA
gccgcccgcctcagcccaaca

SEQ ID NO: 159 - DNA, 21 bp, synthetic construct
  source 1..21: mol_type = other DNA
gcgagctccgccagctcccgc

SEQ ID NO: 160 - DNA, 21 bp, synthetic construct
  source 1..21: mol_type = other DNA
gcgcagatcccggacacccgg

SEQ ID NO: 161 - DNA, 21 bp, synthetic construct
  source 1..21: mol_type = other DNA
gcgccgtgccgagcttcgcta

SEQ ID NO: 162 - DNA, 20 bp, synthetic construct
  source 1..20: mol_type = other DNA
gcgcgcgcaggcgcagagcg

SEQ ID NO: 163 - DNA, 21 bp, synthetic construct
  source 1..21: mol_type = other DNA
gcgtgaagcggaagcaggagc

SEQ ID NO: 164 - DNA, 20 bp, synthetic construct
  source 1..20: mol_type = other DNA
ggacacccggcgggagctgg

SEQ ID NO: 165 - DNA, 20 bp, synthetic construct
  source 1..20: mol_type = other DNA
ggccttagcgaagctcggca

SEQ ID NO: 166 - DNA, 20 bp, synthetic construct
  source 1..20: mol_type = other DNA
ggcggcgccgccgcagatcc

SEQ ID NO: 167 - DNA, 20 bp, synthetic construct
  source 1..20: mol_type = other DNA
ggcggtccctgcgccgccag

SEQ ID NO: 168 - DNA, 20 bp, synthetic construct
  source 1..20: mol_type = other DNA
gggctaggacaggggttgca

SEQ ID NO: 169 - DNA, 21 bp, synthetic construct
  source 1..21: mol_type = other DNA
gtgcatcgccatgttgggctg

SEQ ID NO: 170 - DNA, 21 bp, synthetic construct
  source 1..21: mol_type = other DNA
gaagcatctccgtcgtgaaca

SEQ ID NO: 171 - DNA, 21 bp, synthetic construct
  source 1..21: mol_type = other DNA
gagaagtgacaggaatctgag

SEQ ID NO: 172 - DNA, 21 bp, synthetic construct
  source 1..21: mol_type = other DNA
gcctgccaccattcttcggcc

SEQ ID NO: 173 - DNA, 21 bp, synthetic construct
  source 1..21: mol_type = other DNA
gcgaccctggccgaagaatgg

SEQ ID NO: 174 - DNA, 20 bp, synthetic construct
  source 1..20: mol_type = other DNA
gcggtgggcctgggcgaccc

SEQ ID NO: 175 - DNA, 21 bp, synthetic construct
  source 1..21: mol_type = other DNA
gcgtaacgacgtcacttccgt

SEQ ID NO: 176 - DNA, 21 bp, synthetic construct
  source 1..21: mol_type = other DNA
gctggccgaagaatggtggca

SEQ ID NO: 177 - DNA, 20 bp, synthetic construct
  source 1..20: mol_type = other DNA
ggaaaagggccgggccgctg

SEQ ID NO: 178 - DNA, 20 bp, synthetic construct
  source 1..20: mol_type = other DNA
ggaaggctcagtgcataagt

SEQ ID NO: 179 - DNA, 20 bp, synthetic construct
  source 1..20: mol_type = other DNA
ggacagccgaaaggagcagg

SEQ ID NO: 180 - DNA, 20 bp, synthetic construct
  source 1..20: mol_type = other DNA
ggcggtggcggacagccgaa

SEQ ID NO: 181 - DNA, 21 bp, synthetic construct
  source 1..21: mol_type = other DNA
gtccgtcggaaggttctcatg

SEQ ID NO: 182 - DNA, 21 bp, synthetic construct
  source 1..21: mol_type = other DNA
gtgtagactcgtccctccgga

SEQ ID NO: 183 - DNA, 21 bp, synthetic construct
  source 1..21: mol_type = other DNA
gttatgcactgagccttccgg

SEQ ID NO: 184 - DNA, 21 bp, synthetic construct
  source 1..21: mol_type = other DNA
gttccggagggacgagtctac

SEQ ID NO: 185 - DNA, 21 bp, synthetic construct
  source 1..21: mol_type = other DNA
gactaacgagagaaccgacgg

SEQ ID NO: 186 - DNA, 20 bp, synthetic construct
  source 1..20: mol_type = other DNA
gagaaccgacggaggaccgc

SEQ ID NO: 187 - DNA, 21 bp, synthetic construct
  source 1..21: mol_type = other DNA
gcagcgggctcgagccggcac

SEQ ID NO: 188 - DNA, 21 bp, synthetic construct
  source 1..21: mol_type = other DNA
gcgagcccgctgggcactcgg

SEQ ID NO: 189 - DNA, 21 bp, synthetic construct
  source 1..21: mol_type = other DNA
gcgctaccctcccgccgcccg

SEQ ID NO: 190 - DNA, 21 bp, synthetic construct
  source 1..21: mol_type = other DNA
gctgtgccggctcgagcccgc

SEQ ID NO: 191 - DNA, 20 bp, synthetic construct
  source 1..20: mol_type = other DNA
ggtagctgaagaccagaccg

SEQ ID NO: 192 - DNA, 20 bp, synthetic construct
  source 1..20: mol_type = other DNA
gtgagtgcgcgcgcgtgcgc

SEQ ID NO: 193 - DNA, 21 bp, synthetic construct
  source 1..21: mol_type = other DNA
gaaccaatcattgcagccgcg

SEQ ID NO: 194 - DNA, 21 bp, synthetic construct
  source 1..21: mol_type = other DNA
gaccaatcattgcagccgcgt

SEQ ID NO: 195 - DNA, 21 bp, synthetic construct
  source 1..21: mol_type = other DNA
gagttcccgtgatgccccacg

SEQ ID NO: 196 - DNA, 20 bp, synthetic construct
  source 1..20: mol_type = other DNA
gcagccgcgtggggcatcac

SEQ ID NO: 197 - DNA, 21 bp, synthetic construct
  source 1..21: mol_type = other DNA
gctccggggctataaaagacc

SEQ ID NO: 198 - DNA, 20 bp, synthetic construct
  source 1..20: mol_type = other DNA
ggggcatcacgggaactccg

SEQ ID NO: 199 - DNA, 21 bp, synthetic construct
  source 1..21: mol_type = other DNA
gtgcagccgcgtggggcatca

SEQ ID NO: 200 - DNA, 20 bp, synthetic construct
  source 1..20: mol_type = other DNA
ggacccggggagaaaggacg

SEQ ID NO: 201 - DNA, 20 bp, synthetic construct
  source 1..20: mol_type = other DNA
gccgcggtatataagccggc

SEQ ID NO: 202 - DNA, 20 bp, synthetic construct
  source 1..20: mol_type = other DNA
gccgaggccgcgcggactgg

SEQ ID NO: 203 - DNA, 21 bp, synthetic construct
  source 1..21: mol_type = other DNA
gtctgccccacccggagacac

SEQ ID NO: 204 - DNA, 20 bp, synthetic construct
  source 1..20: mol_type = other DNA
gttcctgaggtggagaacgg

SEQ ID NO: 205 - DNA, 20 bp, synthetic construct
  source 1..20: mol_type = other DNA
gccaataacaagaaggaagt

SEQ ID NO: 206 - DNA, 20 bp, synthetic construct
  source 1..20: mol_type = other DNA
gcggcgcgaagcgctggtgg

SEQ ID NO: 207 - DNA, 20 bp, synthetic construct
  source 1..20: mol_type = other DNA
gactgaattatcctctcggt

SEQ ID NO: 208 - DNA, 20 bp, synthetic construct
  source 1..20: mol_type = other DNA
ggcctcgcttcctgccggcg

SEQ ID NO: 209 - DNA, 20 bp, synthetic construct
  source 1..20: mol_type = other DNA
ggtccccgtcccgccggggg

SEQ ID NO: 210 - DNA, 20 bp, synthetic construct
  source 1..20: mol_type = other DNA
gcaacggtgtaccgcggatc

SEQ ID NO: 211 - DNA, 20 bp, synthetic construct
  source 1..20: mol_type = other DNA
gtggttcacaaccggaccca

SEQ ID NO: 212 - DNA, 21 bp, synthetic construct
  source 1..21: mol_type = other DNA
ggtggttcggtttgcgtggcc

SEQ ID NO: 213 - DNA, 20 bp, synthetic construct
  source 1..20: mol_type = other DNA
gctgggcggacgttgggata

SEQ ID NO: 214 - DNA, 20 bp, synthetic construct
  source 1..20: mol_type = other DNA
ttgaacggagacacgatacc

SEQ ID NO: 215 - DNA, 20 bp, synthetic construct
  source 1..20: mol_type = other DNA
atggagccacaacacccacc

SEQ ID NO: 216 - DNA, 20 bp, synthetic construct
  source 1..20: mol_type = other DNA
ggaaacacaagggacgtctc

SEQ ID NO: 217 - DNA, 20 bp, synthetic construct
  source 1..20: mol_type = other DNA
gctgaaagttacacccaacg

SEQ ID NO: 218 - DNA, 20 bp, synthetic construct
  source 1..20: mol_type = other DNA
ctgccgctaaggatgttggt

SEQ ID NO: 219 - DNA, 20 bp, synthetic construct
  source 1..20: mol_type = other DNA
caagagcaaaatgcagctgg

SEQ ID NO: 220 - DNA, 25 bp, synthetic construct
  source 1..25: mol_type = other DNA
ttccccgagccactactagccatag

SEQ ID NO: 221 - DNA, 25 bp, synthetic construct
  source 1..25: mol_type = other DNA
cctcttacctcagttacaatttata

SEQ ID NO: 222 - DNA, 20 bp, synthetic construct
  source 1..20: mol_type = other DNA
gaagagacacctgtgagggc

SEQ ID NO: 223 - DNA, 20 bp, synthetic construct
  source 1..20: mol_type = other DNA
ggacaccgattcgtaggacc

SEQ ID NO: 224 - DNA, 20 bp, synthetic construct
  source 1..20: mol_type = other DNA
ggcggatcagatgagggaac

SEQ ID NO: 225 - DNA, 20 bp, synthetic construct
  source 1..20: mol_type = other DNA
cagacagcaatacggaccga

SEQ ID NO: 226 - DNA, 20 bp, synthetic construct
  source 1..20: mol_type = other DNA
acacgcgagagtacctgttg

SEQ ID NO: 227 - DNA, 20 bp, synthetic construct
  source 1..20: mol_type = other DNA
tcatcccgcgacaaatccaa

SEQ ID NO: 228 - DNA, 20 bp, synthetic construct
  source 1..20: mol_type = other DNA
cattaacggaatggcaggcg

SEQ ID NO: 229 - DNA, 20 bp, synthetic construct
  source 1..20: mol_type = other DNA
tgcccggataaaggcaaagt

SEQ ID NO: 230 - DNA, 26 bp, synthetic construct
  source 1..26: mol_type = other DNA
tatcttgtggaaaggacgaaacaccg

SEQ ID NO: 231 - DNA, 29 bp, synthetic construct
  source 1..29: mol_type = other DNA
gtttaagagctatgctggaaacagcatag

SEQ ID NO: 232 - DNA, 22 bp, synthetic construct
  source 1..22: mol_type = other DNA
ctacacgacgctcttccgatct

SEQ ID NO: 233 - DNA, 54 bp, synthetic construct
  source 1..54: mol_type = other DNA
gtgactggagttcagacgtgtgctcttccgatcttgtggaaaggacgaaacacc

SEQ ID NO: 234 - DNA, 49 bp, synthetic construct
  source 1..49: mol_type = other DNA
aatgatacggcgaccaccgagatctacactctttccctacacgacgctc

SEQ ID NO: 235 - DNA, 47 bp, synthetic construct
  source 1..47: mol_type = other DNA
  misc_difference 25: note = a, c, t, g, unknown, or other
  misc_difference 26: note = a, c, t, g, unknown, or other
  misc_difference 27: note = a, c, t, g, unknown, or other
  misc_difference 28: note = a, c, t, g, unknown, or other
  misc_difference 29: note = a, c, t, g, unknown, or other
  misc_difference 30: note = a, c, t, g, unknown, or other
  misc_difference 31: note = a, c, t, g, unknown, or other
  misc_difference 32: note = a, c, t, g, unknown, or other
caagcagaagacggcatacgagatnnnnnnnngtgactggagttcag

SEQ ID NO: 236 - DNA, 13 bp, Homo sapiens
  source 1..13: mol_type = genomic DNA
gtcmttatctcct